(19) United States
(12) Patent Application Publication
(10) Pub. No.: US 2024/0142464 A1
(43) Pub. Date: May 2, 2024

(54) METHODS, DEVICE, KIT AND COMPOSITIONS FOR DETECTING FELINE LUNGWORM

(71) Applicant: Idexx Laboratories, Inc., Westbrook, ME (US)

(72) Inventors: Raghavan U.M. Palaniappan, Westbrook, ME (US); Ariel Carruth, Westbrook, ME (US)

(21) Appl. No.: 18/189,874

(22) Filed: Mar. 24, 2023

Related U.S. Application Data
(60) Provisional application No. 63/323,558, filed on Mar. 25, 2022.

(51) Int. Cl. G01N 33/68; A61K 39/00

(52) U.S. Cl. CPC G01N 33/6854; A61K 39/0003; A61K 2039/552; G01N 2333/43526

(57) ABSTRACT

Compositions, devices, kits and methods for the detection of Aelurostronglylus abstrusus (“A. abstrusus”), a feline lungworm species in mammals, is provided. More particularly, polypeptides and polypeptide compositions, antibodies and antibody compositions, devices, kits, and methods for detecting the presence or absence of A. abstrusus antibodies in a sample from a mammal that may also include one or more other feline lungworms, roundworm, hookworm, and whipworm antigens, for diagnosing A. abstrusus lungworm infection and for detecting, diagnosing and treating A. abstrusus lungworm infection. Compositions including polypeptide or polynucleotide immunogens, vaccines, and methods for treating and/or preventing feline lungworm infections in mammals are also provided.

### CROSS-REFERENCE

This application claims the benefit of priority to U.S. Provisional Application No. 63/323,558, filed Mar. 25, 2022, which is incorporated by reference in its entirety.

### STATEMENT REGARDING SEQUENCE LISTING

A computer readable form the Sequence Listing is filed with this application by electronic submission and is incorporated into this application by reference in its entirety. The sequence listing submitted herewith is contained in the text file created Mar. 21, 2023, entitled “20-1518-WO_Sequence-Listing.xml” and 166,470 bytes in size.

### FIELD OF THE DISCLOSURE

The present disclosure relates to compositions, devices, kits and methods for the detection of Aelurostronglylus abstrusus (“A. abstrusus”), a feline lungworm species in mammals as well as compositions and vaccines including the compositions for treating and/or preventing feline lungworm infections in mammals. More particularly, the present disclosure relates to polypeptides and polypeptide compositions, antibodies and antibody compositions, devices, kits, and methods for detecting the presence or absence of A. abstrusus antibodies in a sample from a mammal that may also include one or more other feline lungworms, roundworm, hookworm, and whipworm antigens as well as polypeptides and polynucleotides and vaccines comprising polypeptides or polynucleotides for treating and/or preventing feline lungworm infections in mammals.

### BACKGROUND OF THE DISCLOSURE

Feline aelurostrongylosis is a parasitic lung disease of veterinary importance. A. abstrusus is the best-known feline lungworm and is regarded as the most prevalent worldwide in domestic cats. Other feline lungworms include Oslerus rostratius (“O. rostratus”), Troglostrongylus brevior (“T. brevior”), Capillaria aerophila and Paragonimus spp. Nematode A. abstrusus has an indirect life cycle which includes felines as a definitive host, terrestrial molluscs as intermediate hosts and small vertebrates such as rodents, birds, lizards and frogs as paratenic hosts. Adult worms are localized in the alveolar ducts and bronchioles of the feline host. The female worm hatches eggs in lung parenchyma and small blood vessels where the first stage (L1) larvae develop. The L1 larvae migrate via the bronchi and trachea to the pharynx where they are coughed up, swallowed and passed through the feces into the environment. The L1 larvae develop into infectious L3 larvae that enters into their immediate hosts which is then picked up by a paratenic host. Ingestion of the paratenic host by a feline host is the most recognized means of lungworm transmission. Once adult worms infest the feline host's lungs, they can cause respiratory signs ranging from minimal respiratory signs to interstitial bronchopneumonia, dyspnea and respiratory distress which can lead to death, especially in young, debilitated or immunosuppressed cats. Such non-specific clinical patterns usually require a high level of clinical awareness of the disease in order to initiate prompt treatment.

In addition to the lack of specific clinical signs, a major problem regarding the treatment and prevention of feline aelurostrongylosis is the absence of clear diagnostic procedures. Current methods for diagnosis of A. abstrusus infections primarily involve microscopic examination of fecal samples, either directly in fecal smears or following concentration of ova and parasites by flotation in density media or by sedimentation. Despite this procedure's high adoption, the method has significant shortcomings. These microscopic methods are time consuming and require specialized equipment. In addition, the accuracy of results of these methods is highly dependent upon the skill and expertise of the operator. For example, the Baermann method is considered standard for A. abstrusus diagnosis and involves larvae recovery from feces. However, the Baermann method can show false-negative results due to the low concentrations of larvae in fecal samples. Moreover, the technique requires specific skill in discriminating between A. abstrusus larvae and from other lungworm larvae as A. abstrusus, O. rostrotus and T. brevior may cause mixed infections as they share the same intermediate and paratenic hosts. Not surprisingly, A. abstrusus infection is often undiagnosed or misdiagnosed on routine fecal examination.

Stool handling is disagreeable and hazardous. Sanitary and inoffensive procedures for processing stool are awkward and often complex. Such procedures may include weighing, centrifuging and storing, and are difficult except in a clinical laboratory equipped with a suitable apparatus, protective equipment, and a skilled technician. Therefore, any reduction in the number of steps required to perform a fecal test and any reduction in contact between test operator and the test material is desirable. Clinical laboratories have been using the immunoassay methods for the detection of various viruses, bacteria and non-helminth parasites and organisms in feces. However, there remains a need for a simple immunoassay method for the detection of an A. abstrusus infection in feces, whole blood or in serum. In addition, there also remains a need for treating and/or preventing feline lungworm, e.g. A. abstrusus infections, in mammals.

### SUMMARY OF THE DISCLOSURE

In one aspect, the disclosure includes polypeptides comprising an epitope of A. abstrusus antigen. In one embodiment, the polypeptides comprise an amino acid sequence of SEQ ID NO:2 (TDX1557), SEQ ID NO:3 (rTDX1557), SEQ ID NO.4 (T2 truncate). SEQ ID NO:5 (T3 truncate), SEQ ID NO:6 (T4 truncate), SEQ ID NO:7 (C10 peptide), SEQ ID NO:8 (C11 peptide), SEQ ID NO: 9 (C12 peptide), SEQ ID NO:10 (D1 peptide), SEQ ID NO:11 (D2 peptide), SEQ ID NO:12 (D3 peptide), SEQ ID NO:13 (D4 peptide), SEQ ID NO:14 (D5 peptide), SEQ ID NO:15 (D6 peptide), SEQ ID NO:16 (D7 peptide), SEQ ID NO:17 (D8 peptide), SEQ ID NO:18 (d678 combo), SEQ ID NO:78 (modified d678), SEQ ID NO:79 (d678-amino extended), SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106 (D8M0), SEQ ID NO:107 (D8M1 peptide), SEQ ID NO:108 (D8M2), SEQ ID NO:109 (D8M3), SEQ ID NO:110 (D8M4), SEQ ID NO: 11 (D8M5), SEQ ID NO: 112 (D8M6), SEQ ID NO:115 (D8M9), SEQ ID NO:116 (D8M10), or a polypeptide comprising XXXWF (SEQ ID NO:117), wherein (a) amino acids W and F at positions 4 and 5 respectively are retained and at least one X at positions 1, 2 and 3 is substituted with a conservative or non-conservative amino acid; (b) amino acids W and F at positions 4 and 5 respectively are retained and conservative amino acid substitutions of X are made at positions 1, 2 and 3; (c) amino acids W and F at positions 4 and 5 respectively are retained, the X at positions 1 and 3 are independently amino acid S, a conservative amino acid or a non-conservative amino acid, and the X at position 2 is amino acid K, a conservative amino acid or a non-conservative amino acid, and (d) amino acid W at position 4 or amino acid F at position 5 is substituted with a conservative amino acid, and wherein the X at positions 1 and 3 are independently amino acid S, a conservative amino acid or a non-conservative amino acid, and the X at position 2 is K, a conservative amino acid or a non-conservative amino acid as listed herein or to a polypeptide including a sequence that is a conservative variant of one of those sequences.

In another aspect, the disclosure includes combination polypeptides comprising one or more polypeptides, each polypeptide comprising an epitope of an A. abstrusus antigen. In one embodiment, the combination polypeptides comprise one or more polypeptides comprising the amino acid sequence SEQ ID NO:2 (TDX1557), SEQ ID NO:3 (rTDX1557). SEQ ID NO:4 (T2 truncate), SEQ ID NO:5 (13 truncate), SEQ ID NO:6 (T4 truncate), SEQ ID NO:7 (C10 peptide), SEQ ID NO:8 (C11 peptide), SEQ ID NO: 9 (C12 peptide), SEQ ID NO:10 (D1 peptide), SEQ ID NO:11 (D2 peptide), SEQ ID NO:12 (D3 peptide), SEQ ID NO:13 (D4 peptide), SEQ ID NO:14 (D5 peptide), SEQ ID NO:15 (D6 peptide), SEQ ID NO:16 (D7 peptide), SEQ ID NO:17 (D8 peptide), SEQ ID NO:18 (d678 combo), SEQ ID NO:78 (modified d678), SEQ ID NO:79 (d678-amino extended), SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO.96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO: 106 (D8M0 peptide), SEQ ID NO:107 (D8M1 peptide), SEQ ID NO:108 (D8M2), SEQ ID NO:109 (D8M3), SEQ ID NO:110 (D8M4), SEQ ID NO:111 (D8M5), SEQ ID NO:112 (D8M6), SEQ ID NO:115 (D8M9), SEQ ID NO:116 (D8M10), or a polypeptide comprising XXXWF (SEQ ID NO:117), wherein (a) amino acids W and F at positions 4 and 5 respectively are retained and at least one X at positions 1, 2 and 3 is substituted with a conservative or non-conservative amino acid; (b) amino acids W and F at positions 4 and 5 respectively are retained and conservative amino acid substitutions of X are made at positions 1, 2 and 3; (c) amino acids W and F at positions 4 and 5 respectively are retained, the X at positions 1 and 3 are independently amino acid S, a conservative amino acid or a non-conservative amino acid, and the X at position 2 is amino acid K, a conservative amino acid or a non-conservative amino acid; and (d) amino acid W at position 4 or amino acid F at position 5 is substituted with a conservative amino acid, and wherein the X at positions 1 and 3 are independently amino acid S, a conservative amino acid or a non-conservative amino acid, and the X at position 2 is K, a conservative amino acid or a non-conservative amino acid. In another embodiment, the combination polypeptide comprise two or more polypeptides having an amino acid sequence of SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO: 12, SEQ ID NO:13, SEQ ID NO:14, SEQ ID NO:15, SEQ ID NO:16, SEQ ID NO:17, SEQ ID NO:78 or SEQ ID NO:79 or a variant thereof that differs only in conservative substitutions and/or modifications. Combination polypeptides comprising at least one epitope of a A. abstrusus antigen having an amino acid sequence of SEQ ID NO:18, SEQ ID NO:78, and SEQ ID NO:79, or a variant thereof that differs only in conservative substitutions and/or modifications. Such combination polypeptides may be prepared either by synthetic means or using recombinant DNA technology.

In another aspect, the disclosure includes antibodies that specifically bind to a polypeptide including all or an antigenic portion of the amino acid sequence that corresponds to one or more of SEQ ID NO:2 (TDX1557), SEQ ID NO:3 (rTDX1557), SEQ ID NO:4 (T2 truncate), SEQ ID NO:5 (T3 truncate), SEQ ID NO:6 (T4 truncate), SEQ ID NO:7 (C10 peptide). SEQ ID NO:8 (C11 peptide), SEQ ID NO: 9 (C12 peptide), SEQ ID NO:10 (D1 peptide), SEQ ID NO:11 (D2 peptide), SEQ ID NO:12 (D3 peptide), SEQ ID NO:13 (D4 peptide), SEQ ID NO:14 (D5 peptide), SEQ ID NO:15 (D6 peptide), SEQ ID NO:16 (D7 peptide), SEQ ID NO:17 (D8 peptide), SEQ ID NO:18 (d678 combo), SEQ ID NO:78 (modified d678), SEQ ID NO:79 (d678-amino extended). SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106 (D8M0), SEQ ID NO:107 (D8M1 peptide), SEQ ID NO:108 (D8M2), SEQ ID NO:109 (D8M3), SEQ ID NO:110 (D8M4), SEQ ID NO:111 (D8M5), SEQ ID NO:112 (D8M6), SEQ ID NO:115 (D8M9), SEQ ID NO:116 (D8M10), or a polypeptide comprising XXXWF (SEQ ID NO:117), wherein (a) amino acids W and F at positions 4 and 5 respectively are retained and at least one X at positions 1, 2 and 3 is substituted with a conservative or non-conservative amino acid; (b) amino acids W and F at positions 4 and 5 respectively are retained and conservative amino acid substitutions of X are made at positions 1, 2 and 3; (c) amino acids W and F at positions 4 and 5 respectively are retained, the X at positions 1 and 3 are independently amino acid S, a conservative amino acid or a non-conservative amino acid, and the X at position 2 is amino acid K, a conservative amino acid or a non-conservative amino acid; and (d) amino acid W at position 4 or amino acid F at position 5 is substituted with a conservative amino acid, and wherein the X at positions 1 and 3 are independently amino acid S, a conservative amino acid or a non-conservative amino acid, and the X at position 2 is K, a conservative amino acid or a non-conservative amino acid, as listed herein, or to a polypeptide including a sequence that is a conservative variant of one of those sequences. In a further aspect, the antibodies specifically bind to antigen from A. abstrusus-infested mammals, but do not specifically bind antigen from mammals infected with hookworm, roundworm, whipworm, heartworm and other feline lungworms.

In another aspect, the disclosure includes antibodies that are obtained by immunization with the polypeptide including all or an antigenic portion of the amino acid sequence that corresponds to one or more of SEQ ID NO:2 (TDX1557), SEQ ID NO:3 (rTDX1557), SEQ ID NO:4 (T2 truncate), SEQ ID NO:5 (T3 truncate), SEQ ID NO:6 (T4 truncate), SEQ ID NO:7 (C10 peptide), SEQ ID NO:8 (C11 peptide), SEQ ID NO: 9 (C12 peptide), SEQ ID NO:10 (D1 peptide), SEQ ID NO:11 (D2 peptide), SEQ ID NO:12 (D3 peptide), SEQ ID NO:13 (D4 peptide), SEQ ID NO:14 (D5 peptide). SEQ ID NO:15 (D6 peptide), SEQ ID NO:16 (D7 peptide), SEQ ID NO:17 (D8 peptide), SEQ ID NO:18 (d678 combo), SEQ ID NO:78 (modified d678), SEQ ID NO:79 (d678-amino extended), SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106 (D8M0), SEQ ID NO:107 (D8M1 peptide), SEQ ID NO:108 (D8M2), SEQ ID NO:109 (D8M3), SEQ ID NO:110 (D8M4), SEQ ID NO:111 (D8M5), SEQ ID NO:112 (D8M6), SEQ ID NO:115 (D8M9), SEQ ID NO:116 (D8M10), or a polypeptide comprising XXXWF (SEQ ID NO:117), wherein (a) amino acids W and F at positions 4 and 5 respectively are retained and at least one X at positions 1, 2 and 3 is substituted with a conservative or non-conservative amino acid; (b) amino acids W and F at positions 4 and 5 respectively are retained and conservative amino acid substitutions of X are made at positions 1, 2 and 3; (c) amino acids W and F at positions 4 and 5 respectively are retained, the X at positions 1 and 3 are independently amino acid S, a conservative amino acid or a non-conservative amino acid, and the X at position 2 is amino acid K, a conservative amino acid or a non-conservative amino acid; and (d) amino acid W at position 4 or amino acid F at position 5 is substituted with a conservative amino acid, and wherein the X at positions 1 and 3 are independently amino acid S, a conservative amino acid or a non-conservative amino acid, and the X at position 2 is K, a conservative amino acid or a non-conservative amino acid or with a polypeptide including a sequence that is a conservative variant of one of those sequences.

Within these related aspects, DNA sequences encoding the above polypeptides, expression vectors comprising these DNA sequences and host cells transformed or transfected with such expression vectors are also provided.

In yet another aspect, the disclosure provides a device for detecting the presence or absence of A. abstrusus antigens from a sample; the device comprising a solid support, wherein the solid support has immobilized thereon one or more antibodies that are capable of specifically binding to a polypeptide that has an amino acid sequence that corresponds to SEQ ID NO:2 (TDX1557), SEQ ID NO:3 (rTDX1557), SEQ ID NO:4 (T2 truncate), SEQ ID NO:5 (T3 truncate), SEQ ID NO:6 (T4 truncate), SEQ ID NO:7 (C10 peptide), SEQ ID NO:8 (C11 peptide), SEQ ID NO: 9 (C12 peptide), SEQ ID NO.10 (D1 peptide), SEQ ID NO:11 (D2 peptide), SEQ ID NO:12 (D3 peptide), SEQ ID NO:13 (D4 peptide), SEQ ID NO:14 (D5 peptide), SEQ ID NO:15 (D6 peptide), SEQ ID NO:16 (D7 peptide), SEQ ID NO:17 (D8 peptide), SEQ ID NO:18 (d678 combo), SEQ ID NO:78 (modified d678), SEQ ID NO:79 (d678-amino extended), SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106 (D8M0), SEQ ID NO:107 (D8M1 peptide), SEQ ID NO:108 (D8M2), SEQ ID NO:109 (D8M3), SEQ ID NO:110 (D8M4), SEQ ID NO:111 (D8M5), SEQ ID NO:112 (D8M6), SEQ ID NO:115 (D8M9), SEQ ID NO: 116 (D8M10), or a polypeptide comprising XXXWF (SEQ ID NO:117), wherein (a) amino acids W and F at positions 4 and 5 respectively are retained and at least one X at positions 1, 2 and 3 is substituted with a conservative or non-conservative amino acid; (b) amino acids W and F at positions 4 and 5 respectively are retained and conservative amino acid substitutions of X are made at positions 1, 2 and 3; (c) amino acids W and F at positions 4 and 5 respectively are retained, the X at positions 1 and 3 are independently amino acid S, a conservative amino acid or a non-conservative amino acid, and the X at position 2 is amino acid K, a conservative amino acid or a non-conservative amino acid; and (d) amino acid W at position 4 or amino acid F at position 5 is substituted with a conservative amino acid, and wherein the X at positions 1 and 3 are independently amino acid S, a conservative amino acid or a non-conservative amino acid, and the X at position 2 is K, a conservative amino acid or a non-conservative amino acid, or an antigenic portion thereof. The device may be, but is not limited to being, for example, an ELISA device, such as a lateral flow immunoassay device or microtiterplate device. Mammalian samples that may be tested for A. abstrusus by the device include, but are not limited to being, blood and related fluids such as serum and plasma, fluids and tissue obtained from pharyngeal swabs, transtracheal aspirates, tracheal swabs, bronchoalveolar lavage fluid, pleural effusions and other materials obtained from the respiratory tract, feces and whole tissue, such as tissue from small intestine, large intestine, cecum, colon, rectum, or other tissue obtained from the gastrointestinal tract, for example. The device further may include, but need not include, one or more reagents for the detection of one or more of the group consisting of: one or more non-A. abstrusus worm parasites, one or more non-worm parasites, one or more viruses, one or more fungi, and one or more bacteria.

In yet another aspect, the disclosure provides a method of detecting the presence or absence of A. abstrusus in a sample. The sample can be obtained from a mammal, such as a canine, feline, porcine, bovine, or human. In one aspect, the method is carried out to test a serum sample for A. abstrusus antigen. The method, however, is not limited to being carried out to test a serum sample. In addition to serum, the sample therefore may be, but is not limited to being blood and related fluids such plasma, fluids and tissue obtained from pharyngeal swabs, transtracheal aspirates, tracheal swabs, bronchoalveolar lavage fluid, pleural effusions and other materials obtained from the respiratory tract, whole tissue, such as tissue from small intestine, large intestine, cecum, colon, rectum, or other tissue obtained from the gastrointestinal tract, for example. In one embodiment, steps of the method include contacting the sample with one or more of the polypeptides of the disclosure; forming antibody polypeptide complexes in the presence of the antibodies if any, in the sample; and detecting the presence or absence of the antibody-polypeptide complexes, if any. In another embodiment, steps of the method include contacting the sample with one or more of the combination polypeptides of the disclosure; forming antibody—combination polypeptide complexes in the presence of the antibodies if any, in the sample; and detecting the presence or absence of the antibody-combination polypeptide complexes, if any. In another embodiment, the steps of the method include contacting the sample with one or more of the antibodies of the disclosure; forming antibody polypeptide complexes in the presence of the A. abstrusus antigens, if any, in the sample; and detecting the presence or absence of the antibody-polypeptide complexes, if any. The method further may include one or more of the optional steps of diagnosing the mammal as either having or not having a A. abstrusus infection, treating a mammal diagnosed has having A. abstrusus infection, and determining whether a nucleic acid from A. abstrusus is present in the same sample that was contacted with the polypeptides for the purpose of detecting the presence or absence of A. abstrusus or in some other sample from the mammal.

The method may also be used to test for environmental contamination with conservative variant of one of those sequences. Environmental samples that may be tested by the device include, but are not limited to soil, decomposing material, or fecal or bodily fluid matter from residential settings including yards, gardens, sand boxes, playgrounds. Testing locations may also include parks, beaches, forests, farms, or other locations exposed to fecal or other bodily material from dogs, cats, or other intermediate and paratenic hosts of A. abstrusus. Feces from indoor and outdoor litter boxes may also be tested.

In yet another aspect, the present disclosure includes a kit for carrying out one or more steps of the method of the disclosure. The kit may optionally include, for example, the device and one or more of the compositions of the present disclosure and instructions for carrying out the method of the present disclosure. The kit may further optionally include, for example, one or more indicator reagents, one or more antibody labeling compounds, one or more antibodies, one or more antigen capture reagents, one or more inhibitors, and one or more wash reagents to be used as part of the device and/or to be used in carrying out the method.

In a further aspect, the present disclosure provides pharmaceutical compositions comprising one or more polypeptides containing one or more epitopes of A. abstrusus antigen or one or more polynucleotides encoding these polypeptides, and a physiologically acceptable carrier and vaccines comprising one or more of the above polypeptides and an adjuvant for enhancement of the immune response.

These and other aspects of the present disclosure will become apparent upon reference to the following detailed description and attached drawings. All references disclosed herein are hereby incorporated by reference in their entirety as if each was incorporated individually.

### BRIEF DESCRIPTION OF THE DRAWINGS

FIG. 1A shows SEQ ID NO:1 which is a nucleotide cDNA sequence deduced from A. abstrusus and the encoded amino acid sequence SEQ ID NO:124.

FIG. 1B shows SEQ ID NO:20 which is a construct for the expression of rTDX1557 (rTDX1557 amino acid sequence is SEQ ID NO:3).

FIG. 1C shows a SDS-PAGE gel analysis of supernatant from a mammalian expression system; and an anti-HIS Immunoblot analysis of (lane 1) a pre-stained marker; (lane 2) recombinant TDX1557 (10 ug/mL); (lane 3) recombinant TDX1557 (5 ug/mL); and (lane 4) recombinant TDX1557 (10 ug/mL). The lanes were probed with mAb HIS tagged with alkaline phosphatase followed by development with BCIP/NBT.

FIG. 2A shows a comparison of ELISA assays with A. abstrusus lysate and TDX1557 (2M2) to larval shedding (Baermann method) in High shedding cat (BigMac).

FIG. 2B shows a comparison of ELISA assays with A. abstrusus lysate and TDX1557 (2M2) to larval shedding (Baermann method) in low shedding cat (Burrito). The X-axis indicates days after post-infection (DPI). Y1 & Y2 axis represent OD Value and Shedding rate, respectively. The cats were infected with L3 larvae, and fecal samples and blood were collected every week from infection. The cats were treated with antiparasitic drugs by Day 90. The larval shedding was performed using the standard Baermann method. ELISA assays with serum samples were performed with larval lysate and rTDX1557. The results indicate that larval shedding is inconsistent, but the ELISA assays with lysate and rTDX1557 show a gradual increase in antibody titer against the worm, and the antibody titer falls after day 90, indicating that the assays respond to treatment.

FIG. 3A shows that no cross-reactivity of anti-TDX1557 (2M2) was found with heartworm antigen and shows SDS-PAGE analysis of A. abstrusus lysate and heartworm lysate.

FIG. 3B shows immunoblot analysis of A. abstrusus lysate and heartworm lysate probed with Rabbit pAB against TDX1557 (Anti-TDX1557). Rabbit pAB against TDX1557 recognized a single band of ˜62 kDa of the A. abstrusus lysate but none of them in heartworm lysate. The positive control is recombinant TDX1557 which also migrates to ˜62 kDA.

FIG. 4 shows that no cross-reactivity of anti-TDX1557 (2M2) was found with roundworm antigen using an immunoblot analysis of A. abstrusus lysate and roundworm lysate probed with Rabbit pAB against TDX1557 (Anti-TDX1557). Rabbit pAB against TDX1557 recognized a single band of ˜62 kDa of the A. abstrusus lysate but none of them in roundworm lysate. The positive control is recombinant TDX1557 which also migrates to ˜62 kDA.

FIG. 5A shows a comparison of high shedding BigMac in the fecal samples versus ELISA with rTDX1557 and A. abstrusus lysate from cat's serum samples. An inconsistent shedding rate was observed with fecal samples, whereas the antibody response against rTDX1557 or A. abstrusus lysate shows steady progress. The antibody response falls after Day 90, with anti-parasitic drugs indicating that the antibody against rTDX1557 or lysate responds to the treatment.

FIG. 5B shows an indirect ELISA of rTDX1557 using serum samples, larval shedding in the fecal samples, and SNAP® results from the serum samples of BigMac collected from different periods in tabulated form.

FIG. 5C shows the photographic results of serum samples collected from high shedding BigMac over a time period (Day 0, 7, 14, 28, 63, 9198, 126, 168, and 182) and subjected to SNAP analysis.

FIG. 5D shows SNAP results of the serum samples from uninfected feline field samples which are also indirect ELISA negative to rTDX1557. Of these 10 samples, 9 of them were found to be negative.

FIG. 6 shows the amino acid sequences for polypeptides rTDX1557 (SEQ ID NO:3) and truncated polypeptides T2 (SEQ ID NO:4), T3 (SEQ ID NO:5), and T4 (SEQ ID NO:6). The sequences include the IgG-like signal sequence, a HIS tag label and ADX18 epitope tag.

FIG. 7 shows the predicted secondary structure of rTDX1557 polypeptide and truncated polypeptides T2, T3 and T4 as well as SDS-PAGE analysis of the expression of each truncated peptides.

FIG. 8 shows the evaluation of truncated versions of 2M2 (TDX1557) using indirect ELISA assay. Recombinant polypeptide 2M2 (TDX1557) and truncated polypeptides T2. T3 and T4 were evaluated in an indirect ELISA using rabbit pAbs against TDX1557, temporal feline serum samples (Burrito DPI 98, French Fry DPI 91) and negative feline serum samples (SPF 16CMF3, SPF 16CSL3). The serially diluted serum samples were tested at different concentrations (0.5, 1.5 and 3 ug/ml) of truncated version of recombinants. The rabbit pAbs against TDX1557 reacted with all four polypeptides. However, the experimentally infected samples did not show any reactivity with the T3 truncated polypeptide, indicating that the significant epitope is present only in the protein's carboxy-terminal. SPF negative controls (SPF 16CMF3 and 16CSL3) show no reactivity to any of the antigens. Based on the antigen concentration and the dilution rate, an increase in titration rate was also observed.

FIG. 9 shows evaluation of truncated versions of 2M2 (TDX1557) using indirect ELISA assay. Recombinant 2M2 and truncated T2. T3 and T4 peptides were evaluated in an indirect ELISA using rabbit pAbs, temporal (Burrito DPI 98, French Fry DPI 91) and negative samples (SPF 16CMF3, SPF 16CSL3). The serially diluted scrum samples were tested at different concentrations (0.5, 1.5 and 3 ug/ml) of truncated version of recombinants (A01 is SEQ ID NO:27; A02 is SEQ ID NO:28; A03 is SEQ ID NO:29; A04 is SEQ ID NO:30; A05 is SEQ ID NO:31; A06 is SEQ ID NO:32; A07 is SEQ ID NO:33; A08 is SEQ ID NO:34; A09 is SEQ ID NO:35; A10 is SEQ ID NO:36; A11 is SEQ ID NO:37; A12 is SEQ ID NO:38; B01 is SEQ ID NO:39; B02 is SEQ ID NO:40; B03 is SEQ ID NO:41; B04 is SEQ ID NO:42; B05 is SEQ ID NO:43; B06 is SEQ ID NO:44; B07 is SEQ ID NO:45; B08 is SEQ ID NO:46; B09 is SEQ ID NO:47; B10 is SEQ ID NO:48; B11 is SEQ ID NO:49; B12 is SEQ ID NO:50; C01 is SEQ ID NO:51; C02 is SEQ ID NO:52; C03 is SEQ ID NO:53; C04 is SEQ ID NO:54; C05 is SEQ ID NO:55; C06 is SEQ ID NO:56; C07 is SEQ ID NO:57; C08 is SEQ ID NO:58; C09 is SEQ ID NO:59; C10 is SEQ ID NO:7; C11 is SEQ ID NO:8; C12 is SEQ ID NO:9; D01 is SEQ ID NO:10; D02 is SEQ ID NO:11; D03 is SEQ ID NO:12; D04 is SEQ ID NO:13; D05 is SEQ ID NO:14; D06 is SEQ ID NO:15; D07 is SEQ ID NO:16; D08 is SEQ ID NO:17; D09 is SEQ ID NO:60; D10 is SEQ ID NO:61; D11 is SEQ ID NO:62; D12 is SEQ ID NO:63; E01 is SEQ ID NO:64; E02 is SEQ ID NO:65; E03 is SEQ ID NO:66; E04 is SEQ ID NO:67; E05 is SEQ ID NO:68; E06 is SEQ ID NO:69; E07 is SEQ ID NO:70; E08 is SEQ ID NO:71; E09 is SEQ ID NO:72; E10 is SEQ ID NO:73; E11 is SEQ ID NO:74; E12 is SEQ ID NO:75; F01 is SEQ ID NO:76; and F02 is SEQ ID NO:27). The rabbit pAbs against TDX1557 reacted with 2M2 and truncated all the four different truncates. However, the experimentally infected samples did not show any reactivity with T3 truncate, indicating that the significant epitope is present only in the protein's carboxy-terminal. SPF negative controls (SPF 16CMF3 and 16CSL3) show no reactivity to any of the antigens. Based on the antigen concentration and the dilution rate, an increase in titration rate was also observed.

FIG. 10 shows the results of an evaluation of a peptide array with TDX1557 ELISA positive naturally infected cat serum samples (8) show significant reactivity to the peptides D1 to D8 and C10-C12. The amino acid sequences of a portion of representative reactive peptides D2 (SEQ ID NO:11), D3 (SEQ ID NO:12), D4 (SEQ ID NO:13); D6 (SEQ ID NO:15), D7 (SEQ ID NO:16), D8 (SEQ ID NO:17), C10 (SEQ ID NO:7), C11 (SEQ ID NO:8), C12 (SEQ ID NO:9) are shown (A01 is SEQ ID NO:27; A02 is SEQ ID NO:28; A03 is SEQ ID NO:29; A04 is SEQ ID NO:30; A05 is SEQ ID NO:31; A06 is SEQ ID NO:32; A07 is SEQ ID NO:33; A08 is SEQ ID NO:34; A09 is SEQ ID NO:35; A10 is SEQ ID NO:36; A11 is SEQ ID NO:37; A12 is SEQ ID NO:38; B01 is SEQ ID NO:39; B02 is SEQ ID NO:40; B03 is SEQ ID NO:41; B04 is SEQ ID NO:42; B05 is SEQ ID NO:43; B06 is SEQ ID NO:44; B07 is SEQ ID NO:45; B08 is SEQ ID NO:46; B09 is SEQ ID NO:47; B10 is SEQ ID NO:48; B11 is SEQ ID NO:49; B12 is SEQ ID NO:50; COI is SEQ ID NO:51; C02 is SEQ ID NO:52; C03 is SEQ ID NO:53; C04 is SEQ ID NO:54; C05 is SEQ ID NO:55; C06 is SEQ ID NO:56; C07 is SEQ ID NO:57; C08 is SEQ ID NO:58; C09 is SEQ ID NO:59; C10 is SEQ ID NO:7; C11 is SEQ ID NO:8; C12 is SEQ ID NO:9; D01 is SEQ ID NO:10; D02 is SEQ ID NO:11; D03 is SEQ ID NO:12; D04 is SEQ ID NO:13; D05 is SEQ ID NO:14; D06 is SEQ ID NO:15; D07 is SEQ ID NO:16; D08 is SEQ ID NO:17; D09 is SEQ ID NO:60; D10 is SEQ ID NO:61; D11 is SEQ ID NO:62; D12 is SEQ ID NO:63; E01 is SEQ ID NO:64; E02 is SEQ ID NO:65; E03 is SEQ ID NO:66; E04 is SEQ ID NO:67; E05 is SEQ ID NO:68; E06 is SEQ ID NO:69; E07 is SEQ ID NO:70; E08 is SEQ ID NO:71; E09 is SEQ ID NO:72; E10 is SEQ ID NO:73; E11 is SEQ ID NO:74; E12 is SEQ ID NO:75; F01 is SEQ ID NO:76; and F02 is SEQ ID NO:27).

FIG. 11 shows an evaluation of a peptide array with experimentally infected cat serum samples which show a positive ELISA assay result with rTDX1557. The serum samples show significant reactivity to the peptides. The amino acid sequences of a portion of representative reactive peptides D2 (SEQ ID NO:11), D3 (SEQ ID NO:12). D4 (SEQ ID NO:13); D6 (SEQ ID NO:15), D7 (SEQ ID NO: 16), D8 (SEQ ID NO:17), C10 (SEQ ID NO:7), C11 (SEQ ID NO:8), C12 (SEQ ID NO:9) are shown. (A01 is SEQ ID NO:27; A02 is SEQ ID NO:28; A03 is SEQ ID NO:29; A04 is SEQ ID NO:30; A05 is SEQ ID NO:31; A06 is SEQ ID NO:32; A07 is SEQ ID NO:33; A08 is SEQ ID NO:34; A09 is SEQ ID NO:35; A10 is SEQ ID NO:36; A11 is SEQ ID NO:37; A12 is SEQ ID NO:38; B01 is SEQ ID NO:39; B02 is SEQ ID NO:40; B03 is SEQ ID NO:41; B04 is SEQ ID NO:42; B05 is SEQ ID NO:43; B06 is SEQ ID NO:44; B07 is SEQ ID NO:45; B08 is SEQ ID NO:46; B09 is SEQ ID NO:47; B10 is SEQ ID NO:48; B11 is SEQ ID NO:49; B12 is SEQ ID NO:50; COI is SEQ ID NO:51; C02 is SEQ ID NO:52; C03 is SEQ ID NO:53; C04 is SEQ ID NO:54; C05 is SEQ ID NO:55; C06 is SEQ ID NO:56; C07 is SEQ ID NO:57; C08 is SEQ ID NO:58; C09 is SEQ ID NO:59; C10 is SEQ ID NO:7; C11 is SEQ ID NO:8; C12 is SEQ ID NO:9; D01 is SEQ ID NO:10; D02 is SEQ ID NO:11; D03 is SEQ ID NO:12; D04 is SEQ ID NO:13; D05 is SEQ ID NO:14; D06 is SEQ ID NO:15. D07 is SEQ ID NO:16; D08 is SEQ ID NO:17; D09 is SEQ ID NO:60; D10 is SEQ ID NO:61; D11 is SEQ ID NO:62; D12 is SEQ ID NO:63; E01 is SEQ ID NO:64; E02 is SEQ ID NO:65; E03 is SEQ ID NO:66; E04 is SEQ ID NO:67; E05 is SEQ ID NO:68; E06 is SEQ ID NO:69; E07 is SEQ ID NO:70; E08 is SEQ ID NO:71; E09 is SEQ ID NO:72; E10 is SEQ ID NO:73; E11 is SEQ ID NO:74; E12 is SEQ ID NO:75; F01 is SEQ ID NO:76; and F02 is SEQ ID NO:27).

FIG. 12 shows a comparison of immunogenicity of D6. D7 and D8 peptides (D6—KNLTEMATRASKSWF is SEQ ID NO:15; D7—MATRASKSWFDELKK is SEQ ID NO: 16), D8—SKSWFDELKKFGVPP is SEQ ID NO:17), A. abstrusus lysate and rTDX1557 via ELISA assay using rabbit pAB to rTDX1557. Peptides D6, D7, and D8 detected the rabbit pAB to rTDX1557 early, indicating its diagnostic utility for infection. ELISA assay with A. abstrusus larval lysates and rTDX1557 show a gradual increase in antibody titer. The antibody titer falls after D90 treatment with antiparasitic drug.

FIG. 13 shows a predicted secondary structure for 2M2 polypeptide (SEQ ID NO:124) using Protter software. Based on the predicted structure, 2M2 polypeptide is a secretory protein. An alignment of the amino acid sequences of immunogenic peptides D6, D7, and D8 (D6 is SEQ ID NO:15; D7 is SEQ ID NO: 16), D8 is SEQ ID NO:17) relative to D678 peptide (CPAQKNLTEMATRASKSWFDELKKFG is SEQ ID NO:18) is shown and the overlapping sequence is underlined. The portion of the 2M2 peptide sequence corresponding to the D678 polypeptide is circled

FIG. 14 shows a comparison of immunogenicity of the D678 polypeptide, A. abstrusus lysate and rTDX1557 via ELISA assay using rabbit pAB to rTDX1557. D678 polypeptide, A. abstrusus lysate and rTDX1557 were coating onto plates, washed. Cat serum from cats Big Mac, Burrito, chicken Nugget French Fry, Taco and Tortilla were then applied to the plate. The plate was washed and rabbit pAB was then applied to the plates. A secondary antibody was used for detection. The D678 peptide showed reactivity similar to A. abstrusus lysate.

FIG. 15 shows a schematic representation of extended D678 synthetic peptide sequences for 2M2, 2M2 amino 2Cys, D678 (SEQ ID NO:78), D678 carboxyl (SEQ ID NO:80) and D678 amino (SEQ ID NO:79).

FIG. 16A shows the results of evaluation of the immunogenicity of the extended peptides D678, D678 carboxyl and D678 amino via indirect ELISA assay using rabbit pAbs to rTDX1557 and serum samples from temporal and SPF cats.

FIG. 16B shows schematic representation of the truncated 2M2 polypeptides 2M2 Amino 2Cys, CpepC, T4 WO IDR, T4 C2S, T4 1A, T4 1B, and T4 1C and extended peptides D678, D678 carboxyl and D678 amino.

FIG. 16C shows SEQ ID NO.2 and the construction of the 2M2 full (TDX1557), 2M2 T2 (TDX1637), 2M2 T3 (TDX1674), 2M2 T4 (TDX1675), 2M2 amino2cys, 2M2 CpepC and 2M2 Wo epitope as well as four Cys-Ser variants of 2M2 T4. The four Cys-Ser variants are 2M2 T4 WO IDR, 2M2 T4 1A, 2M2 T4 1B, and 2M2 T4 1C.

FIG. 16 D shows the amino acid sequences for the Cys-Ser variants 2M2 T4 WO IDR (2M2_Amino2Cys_pCDNA34 is SEQ ID NO:118; 2M2_CpepC_pcDNA34 is SEQ ID NO.119; 2M2_T4C2S is SEQ ID NO:120), 2M2 T4 1A (SEQ ID NO:121), 2M2 T4 1B (SEQ ID NO:122), and 2M2 T4 1C (SEQ ID NO:123).

FIG. 16E shows the results of evaluation of the immunogenicity of truncates 2M2 amino2cys and 2M2 CpepC (2M2_Amino2Cys_pCDNA34 is SEQ ID NO:118; 2M2 CpepC_pcDNA34 is SEQ ID NO:119) via indirect ELISA assay using rabbit pAbs to rTDX1557 and serum samples from temporal and SPF cats. No reactivity was seen with samples from temporal cats.

FIG. 16F shows the results of evaluation of the immunogenicity of truncates 2M2 T4 C2S (2M2_T4C2S is SEQ ID NO:120) via indirect ELISA assay using rabbit pAbs to rTDX1557 and serum samples from temporal and SPF cats. No reactivity was seen with samples from temporal cats.

FIG. 17 shows an immunoblot analysis of A. abstrusus lysate, rTDX1557 (positive control), and fecal samples from high larvae shedding cat Big Mac at days 36, 64, 141 and 180 probed with rabbit pAB against rTDX1557 (Anti-TDX1557). Rabbit pAB against TDX1557 recognized a single band of ˜62 kDa of the A. abstrusus lysate, rTDX1557 and BigMac fecal sample at days 36, 64 and 141. BigMac was treated with anti-parasitic drug on DPI 90.

FIG. 18 shows the results of an indirect ELISA assay as described in Example 12, testing the immunoreactivity of peptides D7M0-D7M15 and D8M0-D8M10 with pooled sera from three cats naturally infected with A. abstrusus (i.e., field positives). The assays were run in triplicate. The OD values were normalized against the immunoreactivity (OD values) with serum from a Day 0 sample of an experimentally infected cat.

### DETAILED DESCRIPTION OF THE DISCLOSURE

### Introduction

The present disclosure is generally directed to methods, devices, kits and compositions for detecting A. abstrusus in a sample such as serum obtained from a mammal. In particular, the present disclosure relates to A. abstrusus polypeptides and conservative variants thereof, polynucleotides that encode those polypeptides and oligonucleotides that specifically bind to those polynucleotides, antibodies that are raised against and that specifically bind those polypeptides, and methods, devices and kits for detecting A. abstrusus.

The present disclosure provides a superior alternative to these existing microscopic inspection techniques. This is true because the present disclosure provides compositions, devices, kits and methods for detecting the presence or absence of A. abstrusus in a sample from a mammal that: (1) are both easy to use and yield consistently reliable results; (2) allow for the absence or presence of A. abstrusus in a mammal to be confirmed regardless of whether that mammal is infected with hookworm, roundworm, whipworm, heartworm and/or other feline lungworms; and (3) can detect A. abstrusus prior to the time that A. abstrusus larvae first appear in the infected host's feces.

The present disclosure is based in part on the discovery of unexpected properties of compositions of the present disclosure. Specifically, it was determined that an antibody of the present disclosure raised against a polypeptide of the present disclosure can be used to capture and detect A. abstrusus antigens in a mammal, even when the mammal is also infested by one or more of hookworm, roundworm, whipworm, heartworm and/or other feline lungworms. This specificity for A. abstrusus is surprising because whipworms, roundworms, hookworms, heartworms and other feline lungworms all are related nematodes, and an antibody raised against a protein isolated from any one of these worms would be expected to crossreact with one or more of the other worms, host antigens, or other host components.

It was further determined that this antibody can be used to capture and detect A. abstrusus antigens in a mammal as early as 14 days after the mammal is first infected with A. abstrusus. This ability to detect A. abstrusus so soon after infection, and before the appearance of any A. abstrusus larvae in the feces of the infected mammal, is surprising because A. abstrusus larvae generally do not appear in the feces of an infective host until about 5-6 weeks after the host becomes infected.

The present disclosure therefore includes methods, devices, compositions and kits that use antibodies and/or fragments thereof to specifically capture and detect A. abstrusus antigens in a mammal that may also be infested by one or more of roundworm, hookworm, whipworm, heartworm and/or other feline lungworms. The ability of the present disclosure to detect and diagnose A. abstrusus even when one or more other worm types are also present allows the mammal's caregiver the opportunity to optimally select a treatment for ridding A. abstrusus from the mammal. Further, the ability of the present disclosure to, in some cases, detect A. abstrusus as early as 14 days after the mammal is first infected provides the possibility that the caregiver may begin such treatment before the mammal becomes severely sickened by the A. abstrusus. An intervention prior to appearance of larvae in the feces would also greatly reduce or eliminate the possibility that the infestation is spread to other animals.

### Definitions and Uses of Terms

The term “compositions of the disclosure” refers to all of the nucleic acids, polypeptides, antibodies, and mixtures that include one or more of those nucleic acids, polypeptides, and antibodies and one or more other compounds, that can be used to detect the presence or absence of A. abstrusus in a sample obtained from a mammal by carrying out the method of the present disclosure that are explicitly described, implicitly encompassed or otherwise disclosed herein.

“A sample from a mammal” in which A. abstrusus can be detected by the present disclosure includes all bodily components and extracts thereof, such as any fluid, solid, cell or tissue, that are capable of containing A. abstrusus antigen. Exemplary samples therefore include, but are not limited to being, serum and whole tissue, such as blood and blood components such as plasma, fluids and tissue obtained from pharyngeal swabs, transtracheal aspirates, tracheal swabs, bronchoalveolar lavage fluid, pleural effusions and other materials obtained from the respiratory tract, tissue from small intestine, large intestine, cecum, colon, rectum, or other tissue obtained from the gastrointestinal tract, for example. The sample may be taken directly from the mammal or the sample may be taken from anything that has contacted the mammal. For example, the sample may be fresh or decaying fecal droppings from the mammal. As another example, the sample may include soil, dirt, sand, plant material, or any other material that may be mixed with bodily components that may be left behind by a mammal, such as feces, for example. No matter the origin or the content of the sample, this sample sometimes is referred to herein as the “mammalian sample”, the “test sample” or the “sample under test”.

As used herein, “nucleic acid” is synonymous with, and therefore is used interchangeably with, “gene”, “DNA”, “cDNA”, “EST”, “polynucleotide”, “oligonucleotide”, “polynucleic acid”, “RNA” and “mRNA”. A nucleic acid may be in double-stranded form or it may be in single-stranded form. Further, a nucleic acid is either naturally isolated, such as from a whole A. abstrusus or a portion thereof, for example, or it is artificially synthesized, either in a recombinant host organism or by any other artificial means known to the skilled artisan, such as by employing a PCR-based technique, by creating a transgenic organism that synthesizes the nucleic acid, by using a DNA synthesizing machine, or by any another molecular-based technique, for example.

“Polypeptide”, “peptide” and “protein” are synonymous terms that are used interchangeably herein to refer to a polymer of amino acid residues. A polypeptide, peptide and protein of the present disclosure may be either naturally isolated, such as from a whole A. abstrusus or from a portion of A. abstrusus, for example, or artificially synthesized, either in a recombinant host organism or by any other artificial means known to the skilled artisan. A polypeptide comprising an epitope may consist entirely of the epitope or may contain additional sequences. The additional sequences may be derived from the native antigen or may be heterologous, and such sequences may (but need not) be antigenic.

The term “antibody” or “antibody of the present disclosure” refers to any antibody that is able to specifically bind to one or more A. abstrusus antigens, but not to any antigen from hookworm, roundworm, whipworm or heartworm. The antibodies of the present disclosure may be raised against one or more immunogenic polypeptides of the present disclosure. Unless otherwise stated, it is to be understood that the antibody of the present disclosure may include a mixture of two or more different types of antibody. For example, the antibody may be a mixture of two types of antibodies, wherein one of the two types specifically binds to one particular antigen and the other of the two types specifically binds to some other antigen.

The “immunogenic polypeptide of the present disclosure” and, more simply, “the polypeptide of the present disclosure”, is an immunogen against which the antibodies of the present disclosure may be raised and/or which specifically bind to A. abstrusus antibodies present in a sample. All “polypeptides of the present disclosure” are immunogenic and therefore may be used to elicit an immune response in a host animal to produce the antibodies of the present disclosure. Unless otherwise stated, it is to be understood that the polypeptide of the present disclosure may be one component of a mixed composition of a plurality of components.

An “immunogen” is any agent, such as the immunogenic polypeptide of the present disclosure, for example, that is capable of eliciting an immune response in an animal that is exposed to that agent.

The term “A. abstrusus”, as used herein, refers to nematodes such as lungworms of the order Rhabditida. Exemplary lungworms therefore include Dictyocaulus viviparus, Angiostrongylus cantonensis and_Angiostrongylus vasorum. Further, the term “lungworm”, as used herein, does not refer to the entirety of the phylum Nematoda. For example, “lungworm” does not include any member of the genera Ancylostoma, Uncinaria. Necator. Toxocara, Toxascaris, Ascaris or Dirofilaria.

A “lungworm antigen” or a “antigen of lungworm” is any A. abstrusus product that is present in the serum, blood, material from the respiratory tract or feces of a mammal having a lungworm infection and that may be specifically bound by one or more of the antibodies of the disclosure. For example, a lungworm antigen may be, but is not limited to being, one or more of the polypeptides of the disclosure.

A “lungworm antibody” or a “antibody of lungworm” is any lungworm antibody that is present in the serum or blood of a mammal having a A. abstrusus infection and that may be specifically bound by one or more of the polypeptides of the disclosure. For example, a lungworm antibody may be one, but is not limited to being, that binds to one or more of the polypeptides of the disclosure.

“Specific for”, “specifically binds”, and “stably binds” means that a particular composition of the disclosure, such as an antibody, polypeptide, or oligonucleotide of the present disclosure, for example, recognizes and binds to one or more other agents with greater affinity than to at least one other agent. As one example, an antibody of the present disclosure is said to be “specific for”, to “specifically bind”, and to “stably bind” lungworm antigens whenever that antibody is able to recognize and bind to those lungworm antigens with greater affinity than to any other antigens from a non-lungworm parasitic worm. Such binding specificity can be tested using methodology well known in the art, for example, ELISA or a radioimmunoassay (RIA). Based on information observed regarding the binding specificity of a particular composition of the disclosure, the method of the present disclosure can be carried out under conditions that allow that composition to bind to (and therefore to allow the detection of such binding to) a particular agent or agents, but not to significantly bind other agents, while those conditions are maintained. As one example, the method of the present disclosure can be carried out under conditions that allow an antibody of the present disclosure to bind to (and therefore to allow the detection of such binding to) one or more lungworm antigens present in a particular sample, but not significantly to any hookworm, roundworm, whipworm, heartworm antigen or other lungworm antigen that may be present in that sample.

As another example, a polypeptide of the present disclosure is said to be “specific for”, to “specifically bind”, and to “stably bind” lungworm antibodies whenever that antibody is able to recognize and bind to those lungworm antigenic polypeptides of the disclosure with greater affinity than to any other antigens from a non-lungworm parasitic worm. Such binding specificity can be tested using methodology well known in the art, for example, ELISA or a radioimmunoassay (RIA). Based on information observed regarding the binding specificity of a particular composition of the disclosure, the method of the present disclosure can be carried out under conditions that allow that composition to bind to (and therefore to allow the detection of such binding to) a particular agent or agents, but not to significantly bind other agents, while those conditions are maintained. As one example, the method of the present disclosure can be carried out under conditions that allow an antigenic polypeptides of the present disclosure to bind to (and therefore to allow the detection of such binding to) one or more lungworm antibodies present in a particular sample, but not significantly to any hookworm, roundworm, whipworm, heartworm antigen or other non-A. abstrusus lungworm that may be present in that sample.

“Detecting lungworm” means detecting one or more lungworm-specific products of A. abstrusus, including one or more of the polypeptides, antibodies and nucleic acids of the present disclosure, or one or more lungworm antigens or lungworm antibodies, for example. The presence of one or more such lungworm products in a sample from a mammal is indicative that the mammal has an A. abstrusus infection, regardless of whether any whole lungworm organism or ovum thereof is also present in that sample. Conversely, the absence of one or more such lungworm products a sample from a mammal is indicative that the mammal does not have an A. abstrusus infection.

“Amino acid” refers to naturally occurring and synthetic amino acids. Amino acid residues are abbreviated as follows: Alanine is A or Ala; Arginine is R or Arg; Asparagine is N or Asn; Aspartic Acid is D or Asp; Cysteine is C or Cys; Glutamic Acid is E or Glu; Glutamine is Q or Gln; Glycine is G or Gly; Histidine is H or His; Isoleucine is I or Ile; Leucine is L or Leu; Lysine is K or Lys; Methionine is M or Met; Phenylalanine is F or Phe; Proline is P or Pro; Serine is S or Ser; Threonine is T or Thr; Tryptophan is W or Trp; Tyrosine is Y or Tyr; and Valine is V or Val. Except where defined otherwise herein, X or Xaa represents any amino acid. Other relevant amino acids include, but are not limited to being, 4-hydroxyproline and 5-hydroxylysine. In all cases, the amino acid sequence of a polypeptide described or otherwise referred to herein is presented in conventional form in that the left-most, or first, amino acid residue of the sequence is the N-terminal residue and the right-most, or last, amino acid residue of the sequence is the C-terminal residue.

A “conservative variant” of any particular nucleic acid sequence includes any sequence having one or more degenerate codon substitutions to that particular nucleic acid sequence, any sequence having one or more nucleotide substitutions to, insertions to, and deletions from that particular nucleic acid sequence, and the complementary sequence of that particular nucleic acid and the conservative variants of that complementary sequence. Conservative variants of a particular nucleic acid sequence preferably have at least about 85% identity, more preferably have at least about 90% identity, and even more preferably at least about 95-99% identity, to that particular nucleic acid sequence. Conservative variants of a particular nucleic acid sequence may be artificially synthesized or they may be isolated in their natural form from an organism, including from a lungworm organism, such as A. abstrusus for example.

A “conservative variant” of any particular polypeptide sequence is any polypeptide having an amino acid sequence that varies from the amino acid sequence of that particular polypeptide but still retains the specific binding properties of that particular polypeptide, such that an antibody of the present disclosure that is raised against the particular polypeptide is capable of specifically binding the variant polypeptide. Therefore, for example, a conservative variant of a particular polypeptide may have one or more amino acid substitutions, deletions, additions, and insertions to that particular polypeptide. For example, a conserved variant of a particular polypeptide may have 30 or fewer, 25 or fewer, 20 or fewer, 15 or fewer, 10 or fewer, or 5 or fewer, conserved amino acid substitutions to that particular polypeptide. Conservative variants of a particular polypeptide preferably, but not essentially, have at least about 80% identity, more preferably have at least about 90% identity, and even more preferably at least about 91-99% identity, to that particular polypeptide. A percent identity for any subject nucleic acid or amino acid sequence (e.g., any of polypeptides described herein) relative to another “target” nucleic acid or amino acid sequence can be determined as follows. First, a target nucleic acid or amino acid sequence of the disclosure can be compared and aligned to a subject nucleic acid or amino acid sequence, using the BLAST 2 Sequences (Bl2seq) program from the stand-alone version of BLASTZ containing BLASTN and BLASTP (e.g., version 2.0.14). The stand-alone version of BLASTZ can be obtained at www.ncbi.nlm.nih.gov. Instructions explaining how to use BLASTZ, and specifically the B12seq program, can be found in the ‘readme’ file accompanying BLASTZ. The programs also are described in detail by Karlin et al. (1990) Proc. Natl. Acad. Sci. 87:2264; Karlin et al. (1990) Proc. Natl. Acad. Sci. 90:5873; and Altschul et al. (1997) Nucl. Acids Res. 25:3389.

Bl2seq performs a comparison between the subject sequence and a target sequence using either the BLASTN (used to compare nucleic acid sequences) or BLASTP (used to compare amino acid sequences) algorithm. Typically, the default parameters of a BLOSUM62 scoring matrix, gap existence cost of 11 and extension cost of 1, a word size of 3, an expect value of 10, a per residue cost of 1 and a lambda ratio of 0.85 are used when performing amino acid sequence alignments. The output file contains aligned regions, of homology between the target sequence and the subject sequence. Once aligned, a length is determined by counting the number of consecutive nucleotides or amino acid residues (i.e., excluding gaps) from the target sequence that align with sequence from the subject sequence starting with any matched position and ending with any other matched position. A matched position is any position where an identical nucleotide or amino acid residue is present in both the target and subject sequence. Gaps of one or more residues can be inserted into a target or subject sequence to maximize sequence alignments between structurally conserved domains (e.g., α-helices, β-sheets, and loops).

The percent identity over a particular length is determined by counting the number of matched positions over that particular length, dividing that number by the length and multiplying the resulting value by 100. For example, if (i) a 500 amino acid target sequence is compared to a subject amino acid sequence. (ii) the B12seq program presents 200 amino acids from the target sequence aligned with a region of the subject sequence where the first and last amino acids of that 200 amino acid region are matches, and (iii) the number of matches over those 200 aligned amino acids is 180, then the 500 amino acid target sequence contains a length of 200 and a sequence identity over that length of 90% (i.e., 180/200×100=90). It will be appreciated that a nucleic acid or amino acid target sequence that aligns with a subject sequence can result in many different lengths with each length having its own percent identity. It is noted that the percent identity value can be rounded to the nearest tenth. For example, 78.11, 78.12, 78.13, and 78.14 is rounded down to 78.1, while 78.15, 78.16, 78.17, 78.18, and 78.19 is rounded up to 78.2. It is also noted that the length value will always be an integer.

Conservative variants of a particular polypeptide sequence may be artificially synthesized or they may be isolated in their natural fom from an organism, including from a lungworm organism, such as A. abstrusus, for example. The skilled artisan will also recognize that these variants include, but are not limited to, those have one or more substitutions of basic amino acid residues, one or more substitutions of acidic amino acid residues, one or more substitutions of polar amino acid residues, one or more substitutions of hydrophobic amino acid residues, one or more substitutions of aromatic amino acid residues, and one or more substitutions of small amino acid residues. (“Basic” amino acid residues are K, R and H. “Acidic” amino acid residues are D and E. “Polar” amino acid residues are N and Q. “Hydrophobic” amino acids are 1. L, and V. “Aromatic” amino acid residues are F. Y, and W. “Small” amino acids are G, S, A, T and M.)

An “epitope,” as used herein, is a portion of an antigenic polypeptide that reacts with sera from A. abstrusus-infected mammals (i.e., an epitope is specifically bound by one or more antibodies within such sera). Epitopes of the polypeptides described in the present application may generally be identified using methods known to those of ordinary skill in the art, such as those summarized in Paul, Fundamental Immunology, 3rd ed., 243-247 (Raven Press, 1993) and references cited therein. For example, a polypeptide derived from a native lungworm antigen, produced by recombinant or chemical synthetic methods may be screened for the ability to react with pooled sera obtained from A. abstrusus-infected mammals. Suitable assays for evaluating reactivity with lungworm-infected sera, such as an enzyme linked immunosorbent assay (ELISA), are described in more detail below, and in Harlow and Lane, Antibodies: A Laboratory Manual, Cold Spring Harbor Laboratory, 1988. An epitope of a polypeptide is a portion that reacts with such antisera at a level that is substantially similar to the reactivity of the full length polypeptide. In other words, an epitope may generate at least about 80%, and preferably at least about 100%, of the response generated by the full length polypeptide in an antibody binding assay (e.g., an ELISA).

Combination polypeptides comprising epitopes of multiple antigenic polypeptides are disclosed. A “combination polypeptide” is a polypeptide in which epitopes of different polypeptides or variants thereof are joined, for example through a peptide linkage, into a single amino acid chain. The amino acid chain thus formed may be either linear or branched. The epitopes may be joined directly (i.e., with no intervening amino acids) or may be joined by way of a linker sequence (e.g., Gly-Cys-Gly) that does not significantly alter the antigenic properties of the epitopes. The polypeptide epitopes may also be linked through non-peptide linkages, such as hetero- or homo-bifunctional agents that chemically or photochemically couple between specific functional groups on the polypeptide epitopes such as through amino, carboxyl, or sulfhydryl groups. Bifunctional agents which may be usefully employed in the combination polypeptides of the present disclosure are well known to those of skill in the art. Epitopes may also be linked by means of a complementary ligand/anti-ligand pair, such as avidin/biotin, with one or more epitopes being linked to a first member of the ligand/anti-ligand pair and then being bound to the complementary member of the ligand/anti-ligand pair either in solution or in solid phase. A combination polypeptide may contain multiple epitopes of polypeptides as described herein and/or may contain epitopes of one or more other lungworm antigens.

### Nucleic Acids and Polypeptides of the Disclosure

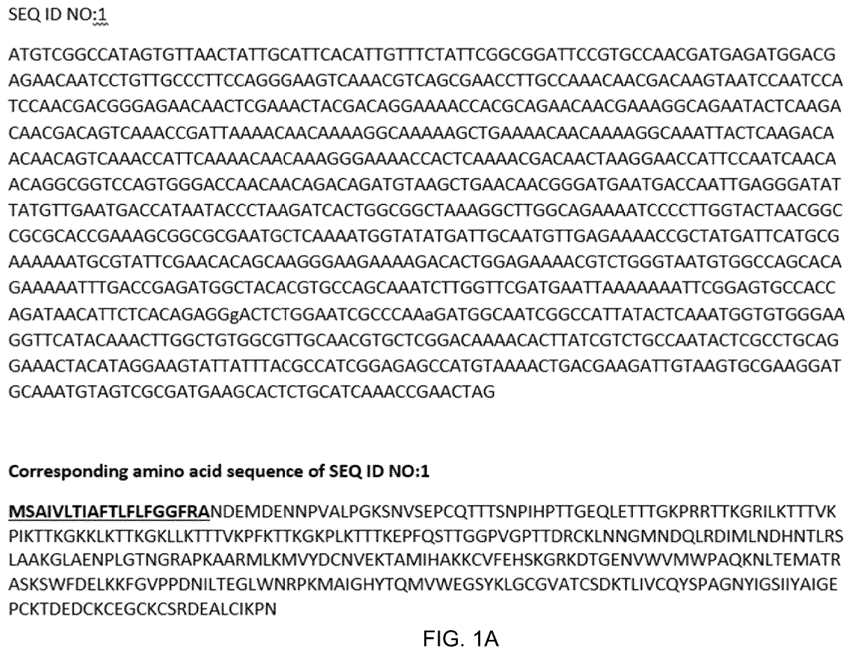

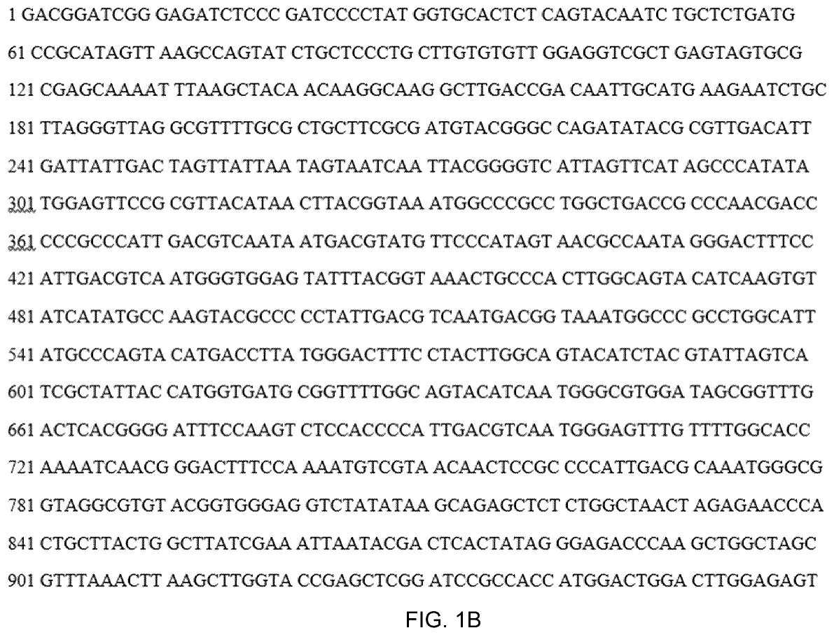

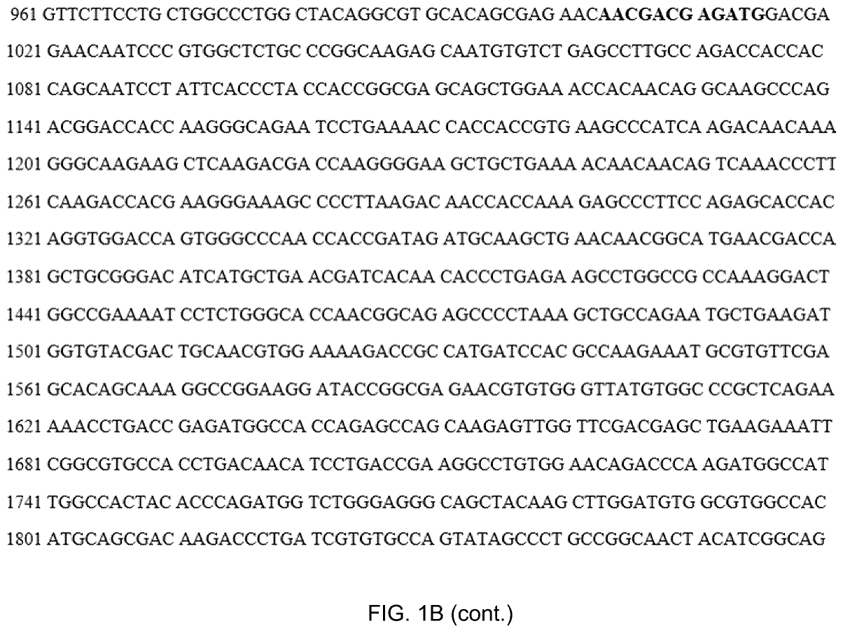

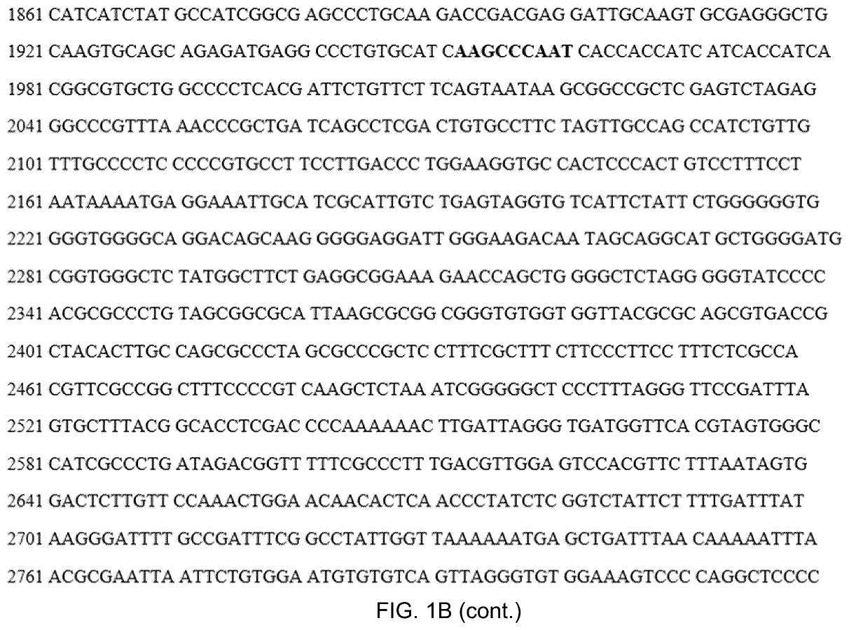

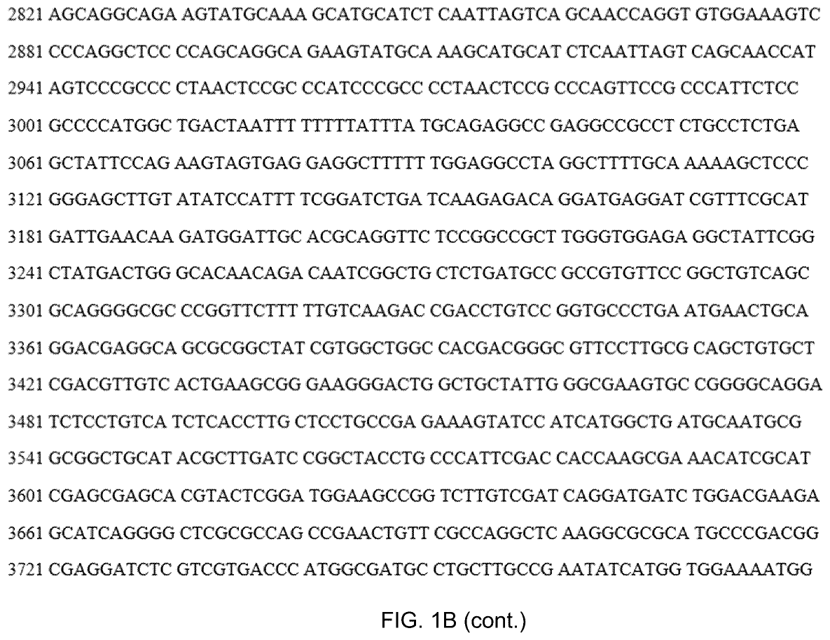

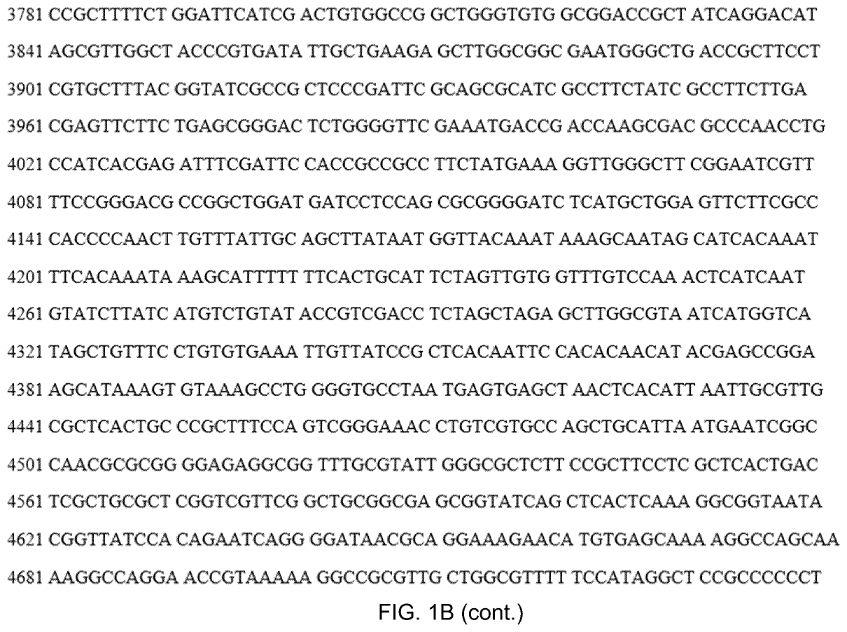

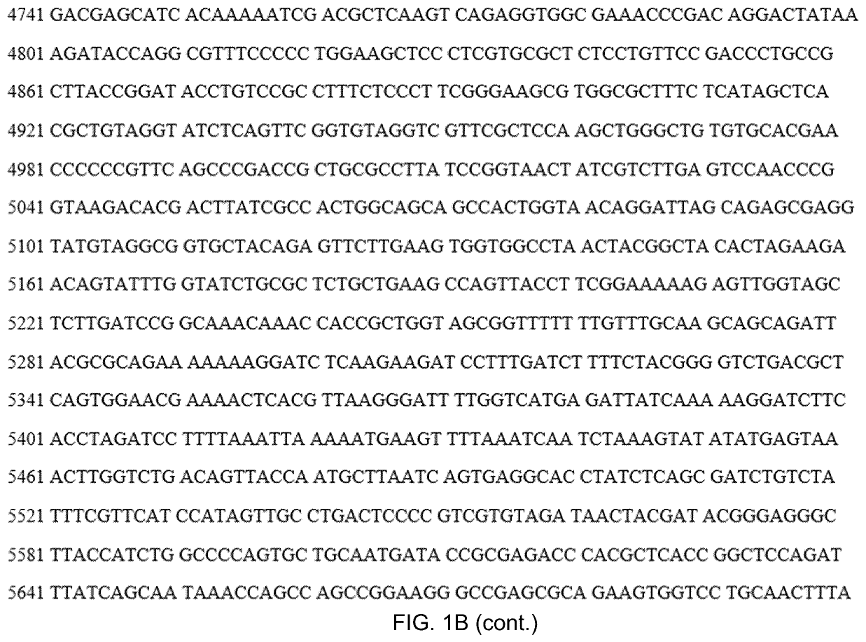

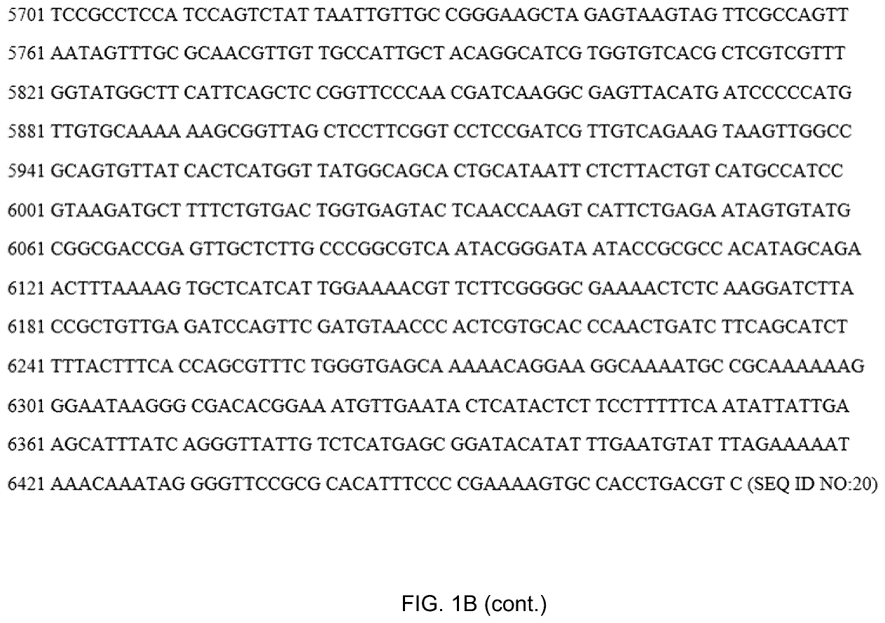

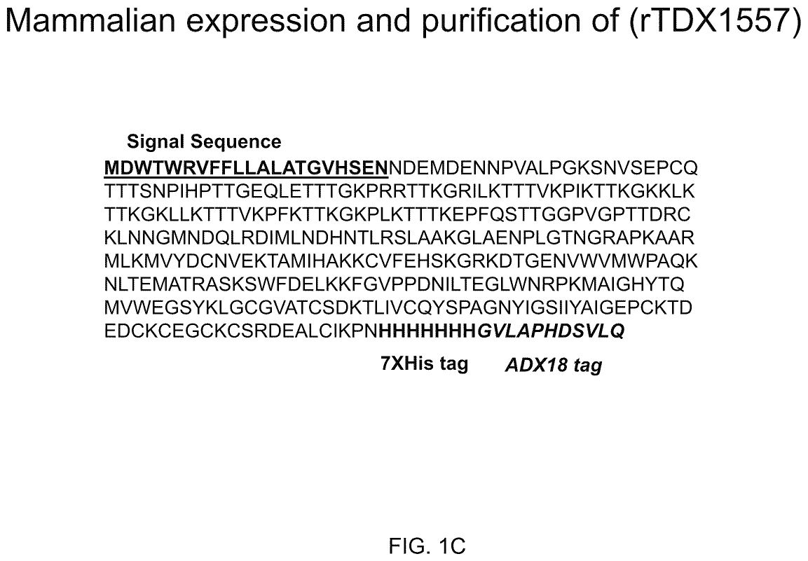

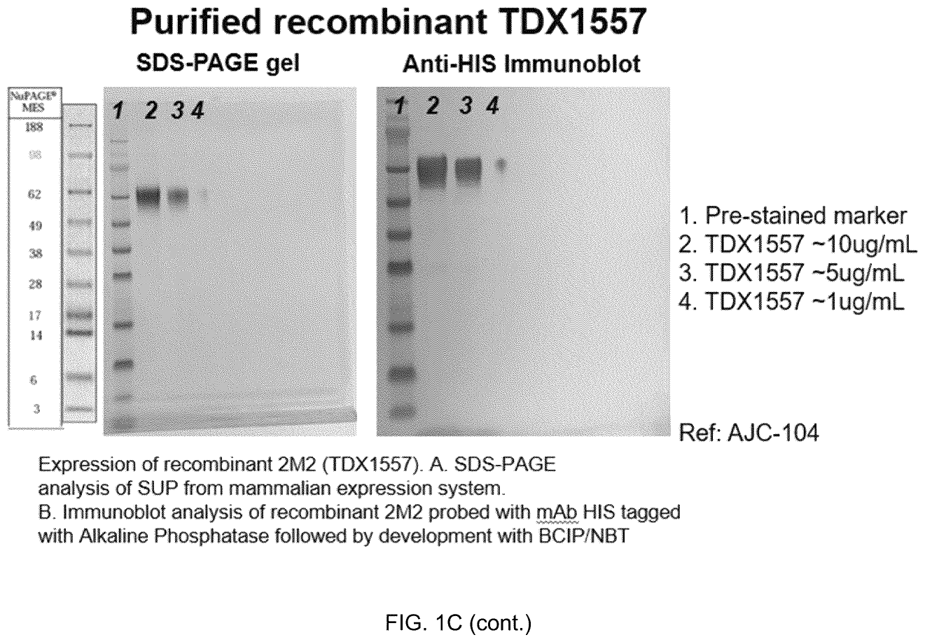

In an attempt to identify compositions that may be used to confirm the presence or absence of lungworm in a mammalian sample, a cDNA library of lambda ZapII was constructed using RNA from L1 larvae of Aelurostrongylus abstrusus. The library was screened with experimentally infected feline serum samples and several positive clones were identified. These positive clones were excised as a plasmid and the insert was sequenced. The DNA sequence of the positive clones were analyzed in NCBI database. As a result of these efforts, an 1020 nucleotide cDNA sequence (SEQ ID NO:1) was deduced from A. abstrusus (this sequence is shown in FIG. 1A and is identified herein as SEQ ID NO:1). (BLAST searches that were carried out using SEQ ID NO:1 indicated that a portion of that sequence is similar to, but is not identical to or substantially identical to, nucleic acid sequence that is predicted to encode a portion of venom allergen/ancylostoma secreted protein-like 10 ATG is the start codon of the nucleotide sequence and TAG is the stop codon of the nucleotide sequence. The first 60 nucleotide sequences (encodes for 20 amino acids) represent the native signal sequences of the secretory protein of A. abstrusus as per Signal IP program.

Analysis of the sequences corresponding to SEQ ID NO:1 indicated that the sequence contains a large ORF. Specifically, as shown in FIG. 1B, the large ORF of SEQ ID NO:1 corresponds to nucleotides 1004 through 1960 of SEQ ID NO:1 and is predicted to encode a polypeptide having the following amino acid sequence:

(SEQ ID NO: 2)
NDEMDENNPVALPGKSNVSEPCQTTTSNPIHPTTGEQLET
TTGKPRRTTKGRILKTTTVKPIKTTKGKKLKTTKGKLLKT
TTVKPFKTTKGKPLKTTTKEPFQSTTGGPVGPTTDRCKLN
NGMNDQLRDIMLNDHNTLRSLAAKGLAENPLGTNGRAPKA
ARMLKMVYDCNVEKTAMIHAKKCVFEHSKGRKDTGENVWV
MWPAQKNLTEMATRASKSWFDELKKFGVPPDNILTEGLWN
RPKMAIGHYTQMVWEGSYKLGCGVATCSDKTLIVCQYSPA
GNYIGSIIYAIGEPCKTDEDCKCEGCKCSRDEALCIKPN.

The polypeptides of the present disclosure may be encoded for by nucleic acids that have a nucleotide sequence that corresponds to all or portions of SEQ ID NO:1 or to all or portions of any conservative variant of those sequences. It is to be understood therefore that the amino acid sequence of the polypeptide of the present disclosure is variable.

For example, the polypeptide of the present disclosure may have an amino acid sequence that corresponds to all or a portion of SEQ ID NO:2 or all or a portion of any conservative variant of SEQ ID NO:2.

In one specific example, a polypeptide of the present disclosure, identified as TDX1557, has the following amino acid sequence:

(SEQ ID NO: 3)
MDWTWRVFFLLALATGVHSENNDEMDENNPVALPGKSNVS
EPCQTTTSNPIHPTTGEQLETTTGKPRRTTKGRILKTTTV
KPIKTTKGKKLKTTKGKLLKTTTVKPFKTTKGKPLKTTTK
EPFQSTTGGPVGPTTDRCKLNNGMNDQLRDIMLNDHNTLR
SLAAKGLAENPLGTNGRAPKAARMLKMVYDCNVEKTAMIH
AKKCVFEHSKGRKDTGENVWVMWPAQKNLTEMATRASKSW
FDELKKFGVPPDNILTEGLWNRPKMAIGHYTQMVWEGSYK
LGCGVATCSDKTLIVCQYSPAGNYIGSIIYAIGEPCKTDE
DCKCEGCKCSRDEALCIKPNHHHHHHGVLAPHDSVLQ

The 319 amino acid residues that are between the IgG-like signal sequence starting from the N-terminal methionine residue of the polypeptide corresponding to SEQ ID NO:3 and the HIS tag starting at amino acid residue 341 represent the amino acid residues 1 through 319 of SEQ ID NO:2. As described in the Example section included herein, the IgG-like signal sequence starting from N-terminal methionine added to the N-terminus of the polypeptide of SEQ ID NO:2 as well as the HIS tag and an epitope tag added to the C-terminus of the polypeptide of SEQ ID NO:2 were artificially added to this polypeptide by carrying out a standard cloning technique. Also as described throughout the Example section, antibody raised against the polypeptide corresponding to SEQ ID NO:3 was useful for detecting A. abstrusus antigen. The polypeptide of SEQ ID NO:3 is referred to interchangeably as 2M2, TDX1557, recombinant TDX1557 or rTDX1557.

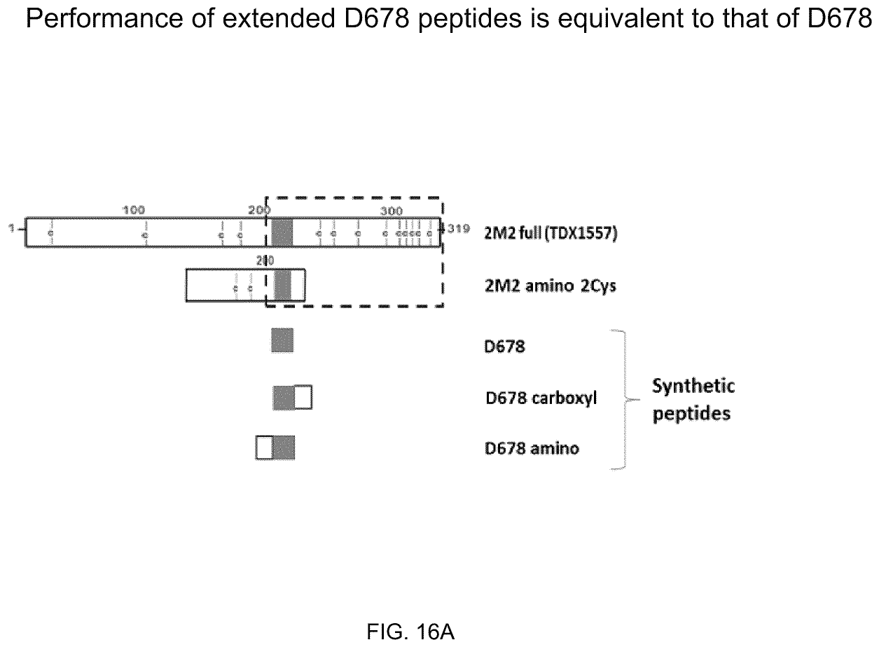

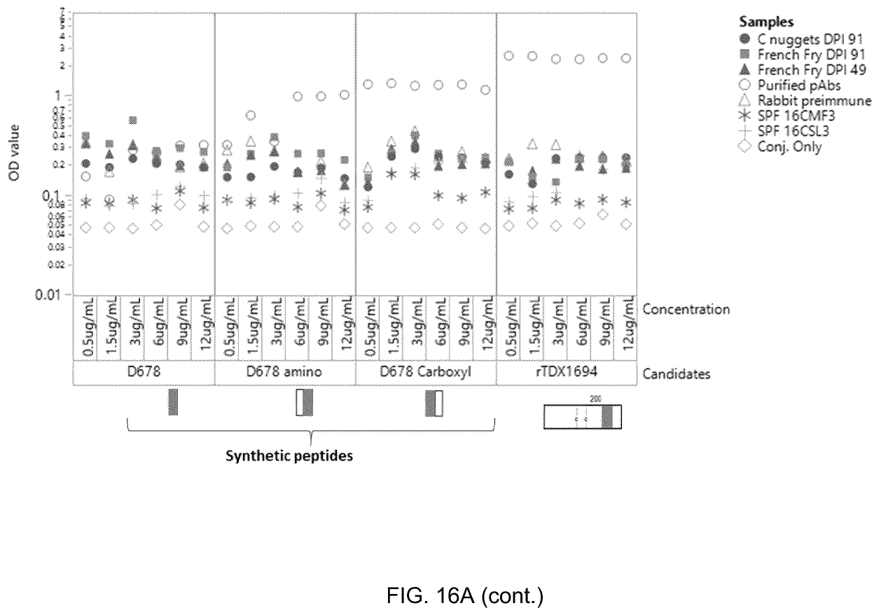

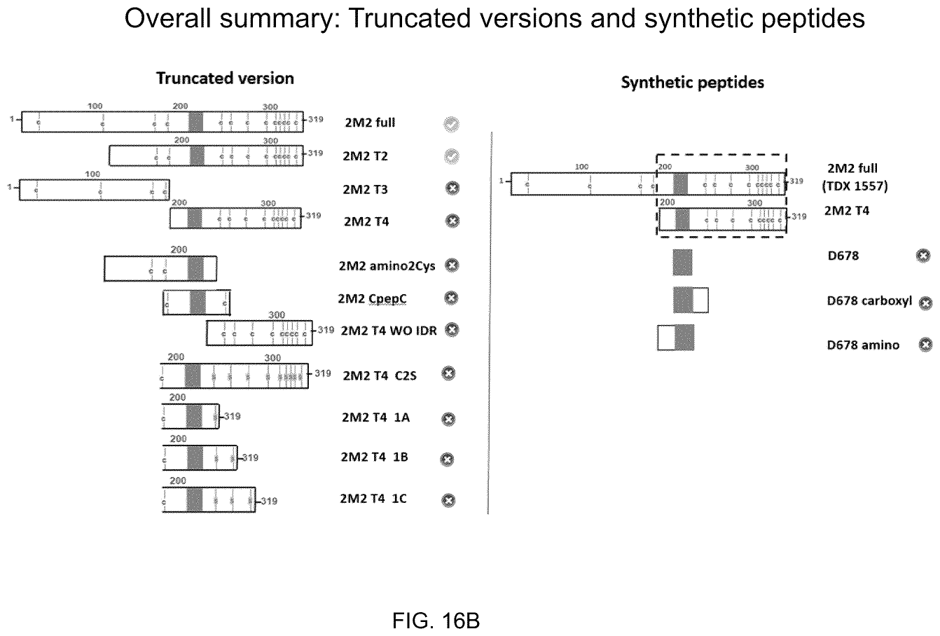

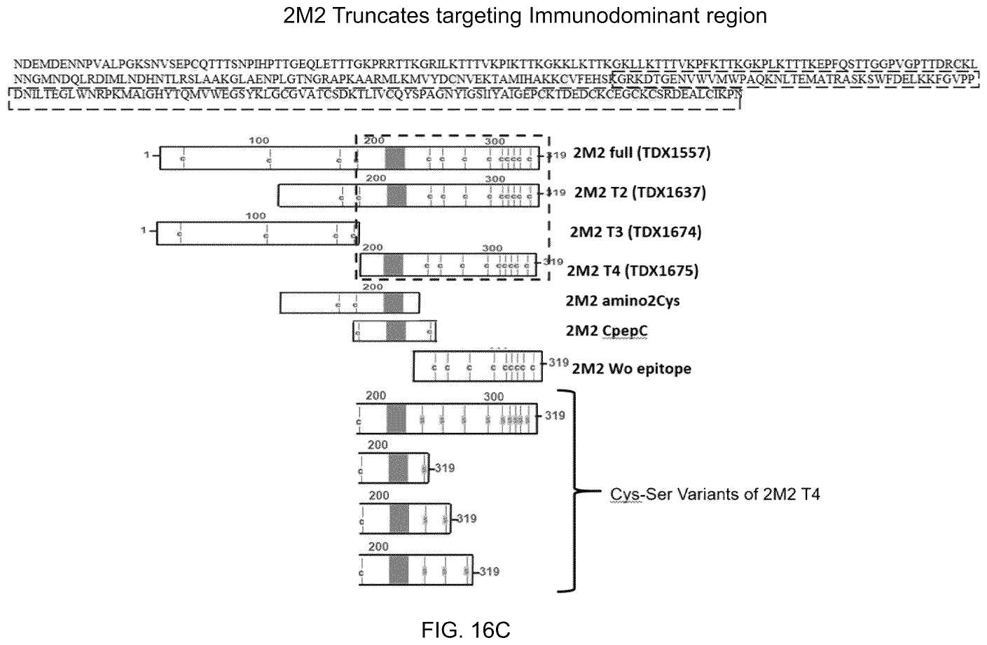

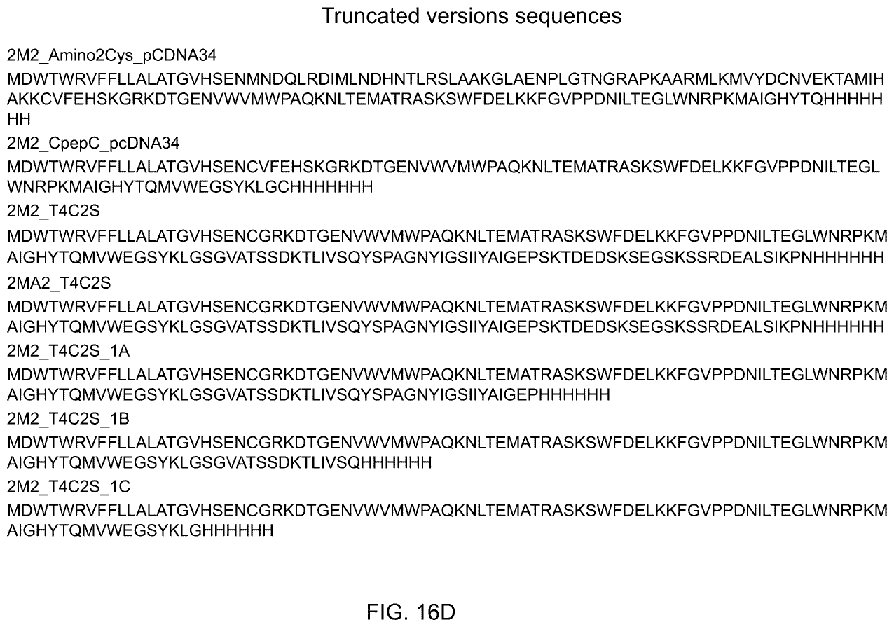

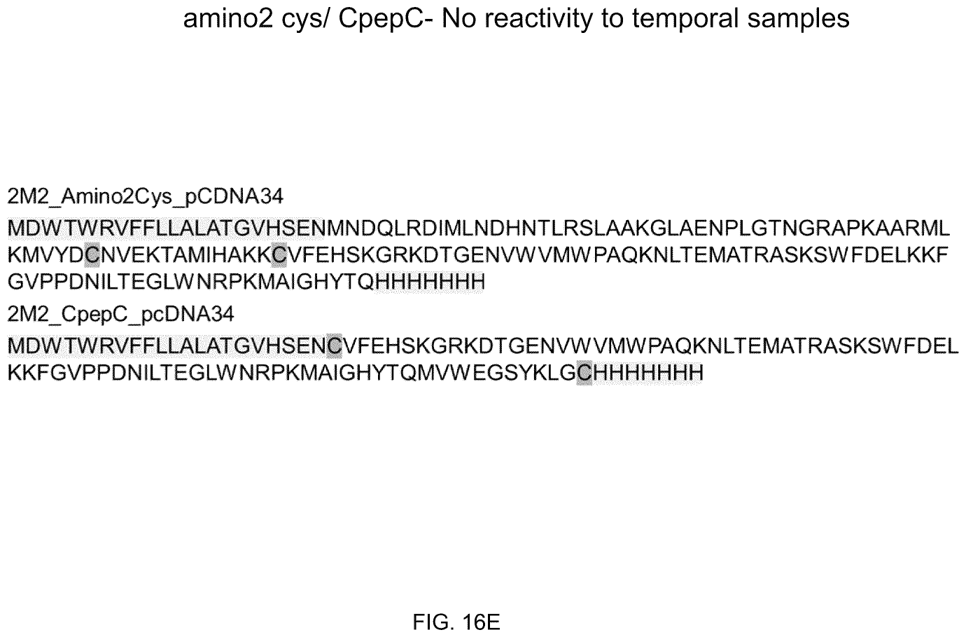

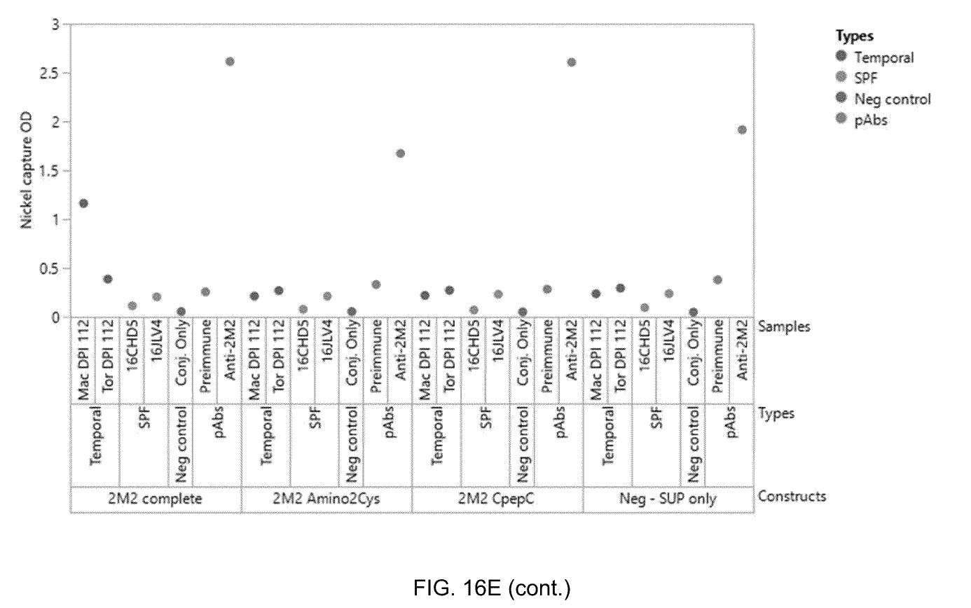

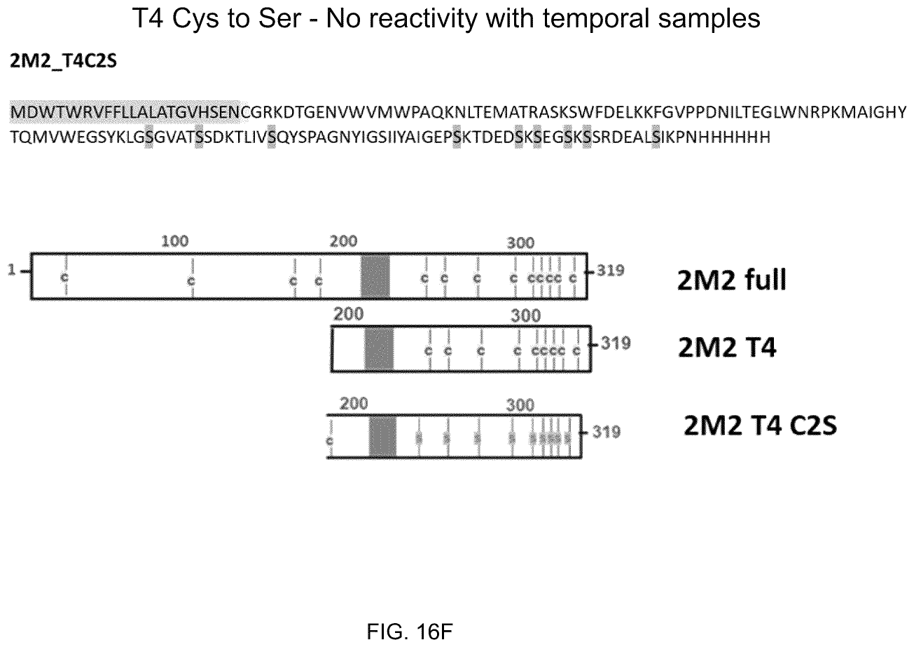

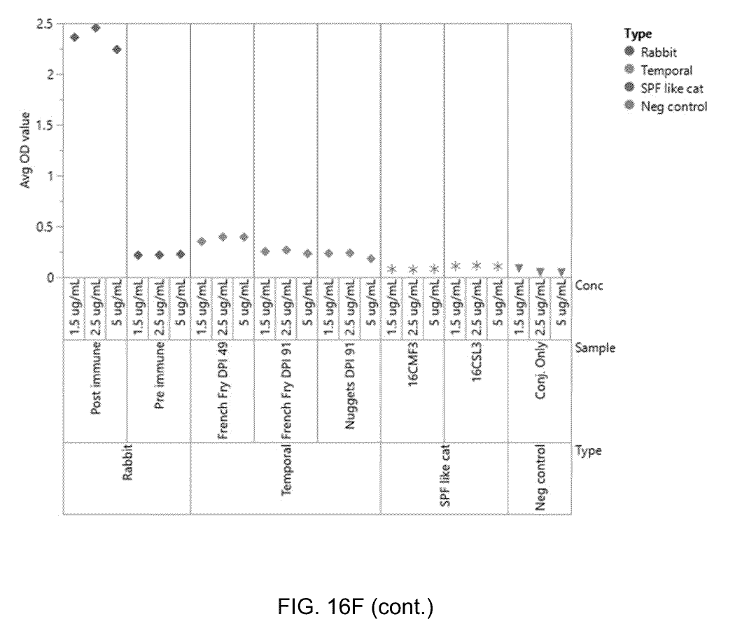

In another specific example, truncated versions of the polypeptide of SEQ ID NO:2 of the disclosure are provided. A schematic representation of the truncated versions of the polypeptide of SEQ ID NO:2 is shown in FIG. 16B. In one specific example, the truncated version of the polypeptide of SEQ ID NO:2 has the following sequence:

2M2_T2 (TDX1637)
(SEQ ID NO: 4)
MDWTWRVFELLALATGVHSENMNDQLRDIMLNDHNTLRSL
AAKGLAENPLGTNGRAPKAARMLKMVYDCNVEKTAMIHAK
KCVFEHSKGRKDTGENVWVMWPAQKNLTEMATRASKSWFD
ELKKFGVPPDNILTEGLWNRPKMAIGHYTQMVWEGSYKLG
CGVATCSDKTLIVCQYSPAGNYIGSIIY AIGEPCKTDED
CKCEGCKCSRDEALCIKPNAAAAAAHHHHHHHGVLAPHDS
VLQ

The 197 amino acid residues that are between the IgG-like signal sequence starting from the N-terminal methionine residue of the polypeptide corresponding to SEQ ID NO:4 and the HIS tag starting at amino acid residue 221 represent a portion of the polypeptide of SEQ ID NO:2, specifically the amino acid residues 123 through 319 of SEQ ID NO:2. As described in the Example section included herein, the IgG-like signal sequence starting from N-terminal methionine added to the N-terminus of the fragment of the polypeptide of SEQ ID NO:2 as well as the HIS tag and an epitope tag added to the C-terminus of the portion of the polypeptide of SEQ ID NO:2 were artificially added by carrying out a standard cloning technique. Also as described throughout the Example section, the polypeptide corresponding to SEQ ID NO:4 was useful for detecting lungworm antibodies present in samples (e.g., serum) taken from A. abstrusus infected mammals. The polypeptide of SEQ ID NO:4 is referred to interchangeably as 2M2T2, TDX1637, or T2 polypeptide.

In another specific example, the truncated version of the polypeptide of SEQ ID NO:2 has the following sequence:

2M2_T3 (TDX1674)
(SEQ ID NO: 5)
MDWTWRVFFLLALATGVHSENNDEMDENNPVALPGKSNVS
EPCQTTTSNPIHPTTGEQLETTTGKPRRTTKGRILKTTTV
KPIKTTKGKKLKTTKGKLLKTTTVKPFKTTKGKPLKTTTK
EPFQSTTGGPVGPTTDRCKLNNGMNDQLRDIMLNDHNTLR
SLAAKGLAENPLGTNGRAPKAARMLKMVYDCNVEKTAMIH
AKKCVFEHSKGRKDTGEAAAHHHHHHHHGVLAPHDSVLQ

The 196 amino acid residues that are between the IgG-like signal sequence starting from the N-terminal methionine residue of the polypeptide corresponding to SEQ ID NO:5 and the HIS tag starting at amino acid residue 221 represent a portion of the polypeptide of SEQ ID NO:2, specifically the amino acid residues 1 through 196 of SEQ ID NO:2. As described in the Example section included herein, the IgG-like signal sequence starting from N-terminal methionine added to the N-terminus of the portion of the polypeptide of SEQ ID NO:2 as well as the HIS tag and an epitope tag added to the C-terminus of the portion of the polypeptide of SEQ ID NO:2 were artificially added by carrying out a standard cloning technique. Also as described throughout the Example section, the polypeptide corresponding to SEQ ID NO:5 was useful for detecting lungworm antibodies present in samples (e.g., serum) taken from A. abstrusus infected mammals. The polypeptide of SEQ ID NO:5 is referred to interchangeably as 2M2T3, TDX1674, or T3 polypeptide.

In one specific example, the truncated version of the polypeptide of SEQ ID NO:2 has the following sequence:

2M2_T4 (TDX1675)
(SEQ ID NO: 6)
MDWTWRVFFLLALATGVHSENGRKDTGENVWVMWPAQKNL
TEMATRASKSWFDELKKFGVPPDNILTEGLWNRPKMAIGH
YTQMVWEGSYKLGCGVATCSDKTLIVCQYSPAGNYIGSII
YAIGEPCKTDEDCKCEGCKCSRDEALCIKPNAAAAAAHHH
HHHHHGVLAPHDSVLQ

The 130 amino acid residues that are between the IgG-like signal sequence starting from the N-terminal methionine residue of the polypeptide corresponding to SEQ ID NO:6 and the HIS tag starting at amino acid residue 155 represent a portion of the polypeptide of SEQ ID NO:2, specifically the amino acid residues 190 through 319 of SEQ ID NO:2. As described in the Example section included herein, the IgG-like signal sequence starting from N-terminal methionine added to the N-terminus of the portion of the polypeptide of SEQ ID NO:2 as well as the HIS tag and an epitope tag added to the C-terminus of the portion of the polypeptide of SEQ ID NO:2 were artificially added by carrying out a standard cloning technique. Also as described throughout the Example section, the polypeptide corresponding to SEQ ID NO:6 was useful for detecting lungworm antibodies present in samples (e.g., serum) taken from A. abstrusus infected mammals. The polypeptide of SEQ ID NO:6 is referred to interchangeably as 2M2_T4, TDX1675, or T4 polypeptide.

In another specific example, the polypeptides of the present disclosure may have an amino acid sequence that corresponds to a portion of SEQ ID NO:2 or a portion of any conservative variant of SEQ ID NO:2. In specific examples, the peptides have the following amino sequences:

C10
(SEQ ID NO: 7)
MVYDCNVEKTAMIHA
C11
(SEQ ID NO: 8)
NVEKTAMIHAKKCVF
C12
(SEQ ID NO: 9)
AMIHAKKCVFEHSKG
D01
(SEQ ID NO: 10)
KKCVFEHSKGRKDTG
D02
(SEQ ID NO: 11)
EHSKGRKDTGENVWV
D03
(SEQ ID NO: 12)
RKDTGENVWVMWPAQ
D04
(SEQ ID NO: 13)
ENVWVMWPAQKNLTE
D05
(SEQ ID NO: 14)
MWPAQKNLTEMATRA
D06
(SEQ ID NO: 15)
KNLTEMATRASKSWF
D07
(SEQ ID NO: 16)
MATRASKSWFDELKK
D08
(SEQ ID NO: 17)
SKSWFDELKKFGVPP

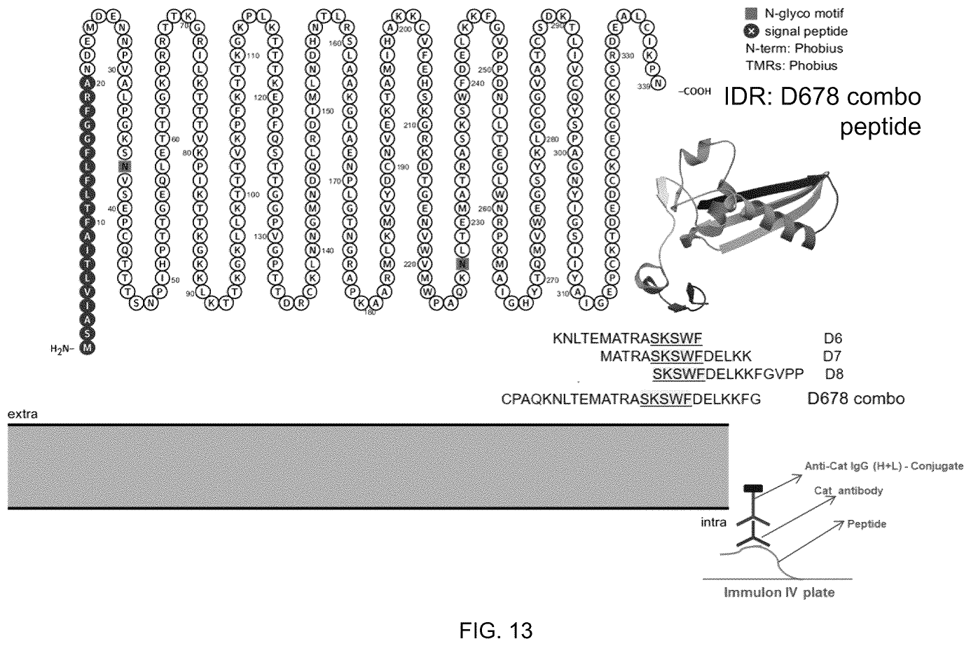

These peptides may be useful for detecting antibodies in samples, for raising antibodies against lungworm antigen, and/or designing antigenic combination polypeptides for detecting A. abstrusus antibodies in a sample. For instance, based on the alignment of polypeptide sequences SEQ ID NO:15. SEQ ID NO:16, and SEQ ID NO:17 corresponding to polypeptides D6. D7 and D8 as shown in FIG. 13, a combination polypeptide of SEQ ID NO:18 was constructed (labeled D678 or D678 combo).

(SEQ ID NO: 18)
CPAQKNLTEMATRASKSWFDELKKFG D678 combo

It is also contemplated that any one or more of SEQ ID NO:2, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16 and SEQ ID NO:17 may be only a portion of a larger polypeptide sequence, and therefore may represent partial sequence of one or more proteins that normally are expressed in A. abstrusus, for example, or one or more polypeptide sequences that are artificially fused to one or more of SEQ ID NO:2, SEQ ID NO:7, SEQ ID NO:8, SEQ ID NO:9, SEQ ID NO: 10, SEQ ID NO:11, SEQ ID NO:12, SEQ ID NO:13, SEQ ID NO:14, SEQ ID NO:15, SEQ ID NO:16 and SEQ ID NO:17. The skilled artisan will recognize that are a variety of techniques exist for artificially fusing two or more polypeptide fragments together.

It is even further contemplated that the polypeptide of the present disclosure may include more than one of the SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16 and SEQ ID NO:17 Also, it is contemplated that the polypeptide of the present disclosure may include a plurality of polypeptide fragments corresponding to one or more of SEQ ID NO:2, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO: 15, SEQ ID NO:16 and SEQ ID NO:17. For example, the polypeptide of the present disclosure may be formed by a plurality of polypeptide fragments corresponding to SEQ ID NO:6 that are fused together. In another example, the polypeptide of the present disclosure may be formed by a plurality of polypeptide fragments corresponding to SEQ ID NO:6 and a plurality of polypeptide fragments corresponding to SEQ ID NO:7 that are fused together in any combination.

Whereas various polypeptides of the present disclosure were expressed and isolated by a specific recombinant technique or were chemically synthesized (as described in the Example section included herein), the skilled artisan will recognize that any of the polypeptides of the present disclosure may be prepared and/or isolated by employing any one or more of a variety of techniques. (See, e.g., Sewald and Jakubke, Peptides: Chemistry and Biology, Wiley Publishing (2002); Peptide Synthesis and Applications (Methods in Molecular Biology) Howl, ed., Humana Press (2005); Jones, Amino Acid and Peptide Synthesis, Oxford University Press (2002), each one of which is incorporated herein by reference in its entirety.) These techniques include those that may be carried out to isolate naturally existing polypeptides having amino acid sequence corresponding to SEQ ID NO: 2, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO: 15, SEQ ID NO:16 and SEQ ID NO: 17 and any naturally occurring variant of those polypeptides. These techniques further include those that may be carried out to artificially generate the polypeptides having amino acid sequence corresponding to SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16 and SEQ ID NO:17 and any conserved variant of those polypeptides. Such variants may be generated, for example, by employing any one or more mutagenesis techniques or by direct synthesis.

The polypeptides of the present disclosure are useful as a reagent for detecting for the presence of anti-A. abstrusus antibodies in a sample. When immobilized onto a substrate and contacted to a sample obtained from a mammal, the immobilized polypeptide are able to specifically bind to, but are not able to specifically bind any antibodies from hookworm, roundworm, or heartworm that may be present in the sample. The polypeptides of the present disclosure are suitable for being used only to capture one or more lungworm antibodies, only to detect one or more lungworm antibodies, or more preferably, to both capture and detect one or more lungworm antibodies.

The polypeptides are also capable of eliciting an immune response in a host animal that is exposed to these polypeptides to produce one or more of the antibodies of the present disclosure. Regardless of the technique by which they are derived, the polypeptides of the present disclosure are preferably prepared in substantially pure form when they are to be used as a reagent for the purpose of raising antibody. Preferably, these polypeptides are at least about 80% pure, more preferably are at least about 90-95% pure, and even more preferably are at least about 99% pure. Exemplary techniques for eliciting an immune response in a host organism and for isolating antibodies therefrom are described herein, but it is to be understood that the present disclosure is not limited to those techniques. The skilled artisan will recognize that there are a plurality of techniques for achieving this same goal without deviating from the scope and spirit of the disclosure.

### Antibodies of the Disclosure

The present disclosure further includes antibodies and antigen-binding fragments thereof that are raised against and that specifically bind all or part of one or more polypeptides of the present disclosure, and also includes compositions that include said antibodies and antigen-binding fragments thereof. When contacted to a sample obtained from a mammal, these antibodies and antigen-binding fragments are able to specifically bind A. abstrusus antigen present in the sample, but are not able to specifically bind any antigen from hookworm, roundworm, or heartworm that may be present in the sample. The antibodies of the present disclosure are suitable for being used only to capture one or more lungworm antigens, only to detect one or more lungworm antigens, or more preferably, to both capture and detect one or more lungworm antigens.

The antibodies of the present disclosure may belong to any antibody class, including for example, IgG, IgM, IgA, IgD and IgE, and may be prepared by any of a variety of techniques known to the skilled artisan. (See. e.g., Dean. Methods Mol. Biol. 80:23-37 (1998); Dean, Methods Mol. Biol. 32:361-79 (1994); Baileg, Methods Mol. Biol. 32:381-88 (1994); Gullick, Methods Mol. Biol. 32:389-99 (1994); Drenckhahn et al. Methods Cell. Biol. 37:7-56 (1993); Morrison, Ann. Rev. Immunol. 10:239-65 (1992); Wright et al. Crit. Rev. Inmunol. 12:125-68(1992); Harlow and Lane, Antibodies: A Laboratory Manual, Cold Spring Harbor Laboratory (1988); and Making and Using Antibodies: A Practical Handbook, Howard and Kaser, eds., CRC Press (2006), each one of which is incorporated herein by reference in its entirety.)

In one technique, the polypeptide of the disclosure is introduced into a host animal, such as into rabbit, mouse, rat, guinea pig, goat, pig, cow, sheep, donkey, dog, cat, chicken, or horse, for example. An enhanced immune response may be elicited in the host animal by associating the polypeptide with a carrier and/or by exposing the host to an adjuvant, but it is to be understood that the present disclosure does not require that the polypeptide be associated with a carrier or that the host be exposed to the adjuvant. An exemplary carrier that may be used for this purpose is bovine serum albumin, bovine thyroglobulin, and soybean trypsin inhibitor. Exemplary adjuvants include Freund's complete or incomplete adjuvant and MDL-TDM adjuvant. Regardless of whether the polypeptide is associated with such a carrier or whether the host is exposed to an adjuvant, booster immunizations optionally may be made with the host animal being bled one or more times thereafter. Polyclonal antibodies that specifically bind the polypeptide may then be purified from antisera obtained from the bleed or bleeds. Such purification may be achieved, for example, by employing affinity chromatography techniques that involve associating the polypeptide to a solid support. Such affinity chromatography techniques are well known by the skilled artisan.

In one embodiment, the antibody of the present disclosure is an antibody that is raised in rabbit by immunizing that host animal with the polypeptide having the amino acid sequence corresponding to SEQ ID NO:3. (Hereinafter, this particular antibody is referred to as “anti-TDX1557”.) A specific technique for producing and isolating anti-TDX1557 pAB is described in the Example section included herein, but the skilled artisan will recognize that the production and isolating of anti-TDX1557 pAB, or any other antibody of the present disclosure, is not limited to that specific technique.

In another embodiment, the antibody of the present disclosure is raised in a host against one or more polypeptides having an amino acid sequence that is a conservative variant of the sequence corresponding to SEQ ID NO:3. In some other embodiments, the antibody of the present disclosure is raised in a host against any one or more polypeptides having an amino acid sequence corresponding to one or more sequences of SEQ ID NO:2 (TDX1557), SEQ ID NO:3 (rTDX1557), SEQ ID NO:4 (T2 truncate), SEQ ID NO:5 (T3 truncate). SEQ ID NO:6 (T4 truncate). SEQ ID NO:7 (C10 peptide), SEQ ID NO:8 (C11 peptide), SEQ ID NO: 9 (C12 peptide), SEQ ID NO:10 (D1 peptide), SEQ ID NO:11 (D2 peptide), SEQ ID NO:12 (D3 peptide), SEQ ID NO:13 (D4 peptide), SEQ ID NO:14 (D5 peptide), SEQ ID NO:15 (D6 peptide), SEQ ID NO:16 (D7 peptide), SEQ ID NO:17 (D8 peptide), SEQ ID NO:18 (d678 combo), SEQ ID NO:78 (modified d678), SEQ ID NO:79 (d678-amino extended), SEQ ID SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106 (D8M0), SEQ ID NO:107 (D8M1 peptide), SEQ ID NO:108 (D8M2). SEQ ID NO:109 (D8M3), SEQ ID NO:110 (D8M4), SEQ ID NO:111 (D8M5), SEQ ID NO:112 (D8M6), SEQ ID NO:115 (D8M9), SEQ ID NO:116 (D8M10), or a polypeptide comprising XXXWF (SEQ ID NO:117), wherein (a) amino acids W and F at positions 4 and 5 respectively are retained and at least one X at positions 1, 2 and 3 is substituted with a conservative or non-conservative amino acid; (b) amino acids W and F at positions 4 and 5 respectively are retained and conservative amino acid substitutions of X are made at positions 1, 2 and 3; (c) amino acids W and F at positions 4 and 5 respectively are retained, the X at positions 1 and 3 are independently amino acid S, a conservative amino acid or a non-conservative amino acid, and the X at position 2 is amino acid K, a conservative amino acid or a non-conservative amino acid; and (d) amino acid W at position 4 or amino acid F at position 5 is substituted with a conservative amino acid, and wherein the X at positions 1 and 3 are independently amino acid S, a conservative amino acid or a non-conservative amino acid, and the X at position 2 is K, a conservative amino acid or a non-conservative amino acid, or one or more polypeptides having an amino acid sequence that is a conservative variant of any of those sequences.

In another embodiment, the antibody of the present disclosure is an antibody that specifically binds one or more polypeptides having the amino acid sequence corresponding to SEQ ID NO:2 (TDX1557), SEQ ID NO:3 (rTDX1557), SEQ ID NO:4 (T2 truncate), SEQ ID NO:5 (T3 truncate), SEQ ID NO:6 (T4 truncate), SEQ ID NO:7 (C10 peptide). SEQ ID NO:8 (C11 peptide), SEQ ID NO: 9 (C12 peptide), SEQ ID NO:10 (D1 peptide), SEQ ID NO:11 (D2 peptide), SEQ ID NO:12 (D3 peptide), SEQ ID NO:13 (D4 peptide), SEQ ID NO:14 (D5 peptide), SEQ ID NO:15 (D6 peptide), SEQ ID NO:16 (D7 peptide), SEQ ID NO:17 (D8 peptide), SEQ ID NO:18 (d678 combo), SEQ ID NO:78 (modified d678), SEQ ID NO:79 (d678-amino extended), SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO: 106 (D8M0), SEQ ID NO:107 (D8M1 peptide), SEQ ID NO:108 (D8M2), SEQ ID NO:109 (D8M3), SEQ ID NO:110 (D8M4), SEQ ID NO:111 (D8M5), SEQ ID NO:112 (D8M6), SEQ ID NO:115 (D8M9), SEQ ID NO:116 (D8M10), or a polypeptide comprising XXXWF (SEQ ID NO:117), wherein (a) amino acids W and F at positions 4 and 5 respectively are retained and at least one X at positions 1, 2 and 3 is substituted with a conservative or non-conservative amino acid; (b) amino acids W and F at positions 4 and 5 respectively are retained and conservative amino acid substitutions of X are made at positions 1, 2 and 3; (c) amino acids W and F at positions 4 and 5 respectively are retained, the X at positions 1 and 3 are independently amino acid S, a conservative amino acid or a non-conservative amino acid, and the X at position 2 is amino acid K, a conservative amino acid or a non-conservative amino acid; and (d) amino acid W at position 4 or amino acid F at position 5 is substituted with a conservative amino acid, and wherein the X at positions 1 and 3 are independently amino acid S, a conservative amino acid or a non-conservative amino acid, and the X at position 2 is K, a conservative amino acid or a non-conservative amino acid, or antigenic portions thereof.

In yet other embodiments, the antibody of the present disclosure specifically binds one or more polypeptides having an amino acid sequence that is a conservative variant of the sequence corresponding to SEQ ID NO:2 (TDX1557). SEQ ID NO:3 (rTDX1557), SEQ ID NO:4 (T2 truncate), SEQ ID NO:5 (T3 truncate), SEQ ID NO:6 (T4 truncate), SEQ ID NO:7 (C10 peptide), SEQ ID NO:8 (C11 peptide), SEQ ID NO: 9 (C12 peptide), SEQ ID NO:10 (D1 peptide), SEQ ID NO:11 (D2 peptide), SEQ ID NO:12 (D3 peptide), SEQ ID NO:13 (D4 peptide), SEQ ID NO:14 (D5 peptide), SEQ ID NO:15 (D6 peptide), SEQ ID NO:16 (D7 peptide), SEQ ID NO:17 (D8 peptide), SEQ ID NO:18 (d678 combo), SEQ ID NO:78 (modified d678), SEQ ID NO:79 (d678-amino extended), SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106 (D8M0), SEQ ID NO:107 (D8M1 peptide), SEQ ID NO:108 (D8M2), SEQ ID NO:109 (D8M3), SEQ ID NO:110 (D8M4), SEQ ID NO:111 (D8M5), SEQ ID NO:112 (D8M6), SEQ ID NO:115 (D8M9), SEQ ID NO:116 (D8M10), or a polypeptide comprising XXXWF (SEQ ID NO:117), wherein (a) amino acids W and F at positions 4 and 5 respectively are retained and at least one X at positions 1, 2 and 3 is substituted with a conservative or non-conservative amino acid; (b) amino acids W and F at positions 4 and 5 respectively are retained and conservative amino acid substitutions of X are made at positions 1, 2 and 3; (c) amino acids W and F at positions 4 and 5 respectively are retained, the X at positions 1 and 3 are independently amino acid S, a conservative amino acid or a non-conservative amino acid, and the X at position 2 is amino acid K, a conservative amino acid or a non-conservative amino acid; and (d) amino acid W at position 4 or amino acid F at position 5 is substituted with a conservative amino acid, and wherein the X at positions 1 and 3 are independently amino acid S, a conservative amino acid or a non-conservative amino acid, and the X at position 2 is K, a conservative amino acid or a non-conservative amino acid.

It is also to be understood that the antibodies of the disclosure may be polyclonal or monoclonal antibodies, single chain antibodies (scFv), chimeric antibodies, and fragments thereof. Monoclonal antibodies that are specific for the polypeptide of interest may be obtained and purified, for example, by preparing cell lines that generate antibodies having the desired specificity to the polypeptide of interest. Cell lines of this kind may be derived from cells of a particular type (e.g., spleen cells) that are isolated from a host animal that had previously been immunized with the polypeptide as described before. In such a case, these cells could then be immortalized, for example, by fusing them with myeloma cells by carrying out any one of a variety of fusion techniques known to the skilled artisan. In one exemplary technique, the cells from the immunized host animal are co-incubated with their fusion partner, e.g., the myeloma cells, in the presence of a detergent for a short period of time before being plated on a medium that supports the growth of hybrid cells (but not the myeloma fusion partner). Such selection may be achieved, for example, by using hypoxanthine, aminopterin, and thymidine (HAT). When hybrid cells emerge during selection, in perhaps one or two weeks after commencing the selection process, single hybrid colonies (and their supernatants) are tested for their ability to bind the polypeptide or polypeptides against which the host animal was immunized. Hybrid colonies having the most optimal binding specificity would represent the best candidates from which monoclonal antibodies may be isolated. These monoclonal antibodies, for example, may be isolated directly from the supernatant (i.e., medium) in which these colonies are grown by employing any one of a variety techniques known to the skilled artisan.

The antibodies of the disclosure also may be a single chain antibody (scFv), or an antigen binding fragment of an antibody. Antigen-binding fragments of antibodies are a portion of an intact antibody comprising the antigen binding site or variable region of an intact antibody, wherein the portion is free of the constant heavy chain domains of the Fc region of the intact antibody. Examples of antibody fragments include Fab, Fab′, Fab′-SH, F(ab′)₂ and Fᵥ fragments. In addition to production and purification from animals or mammalian cells, antibodies, antibody fragments, or non-antibody scaffolds can be selected based upon various in vitro technologies, including phage display, ribosomal display or bacterial display.

Antibodies, including secondary antibodies, may be labeled with any type of label known in the art, including, for example, fluorescent, chemiluminescent, radioactive, enzymes, colloidal particles, radioisotopes and bioluminescent labels. In various embodiments of the disclosure, the one or more of the antibodies of the disclosure are labeled with an enzyme, a colloidal particle, a radionuclide or a fluorophore. The particulate label can be, for example, a colored latex particle, dye sol, or gold sol conjugated to an antibody.

### Methods, Devices and Kits of the Disclosure

### Devices and Kits of the Disclosure

The present disclosure, in one aspect, is a device for the detection of A. abstrusus infection in a mammal, such as a canine, feline, porcine, bovine, or human, for example. The device is arranged to aid in the detection of the presence or absence of A. abstrusus antibody using the polypeptides of the disclosure (or A. abstrusus antigen using the antibodies of the disclosure) in a sample from a mammal that may also be infected with one or more other worm parasites, including hookworm, roundworm, whipworm and heartworm.

In one aspect, the device includes a solid support, wherein one or more polypeptides (or antibodies) of the disclosure are immobilized on the solid support. The solid support may be, but is not limited to being, the inner, bottom surface of a well of a microtiter plate or a substrate that is included as part of a lateral flow device, for example. An exemplary microtiter plate is an Immulon 1B 96-well plate (which is commercially available from Thermo Scientific of Milford, MA), but it is to be understood that the skilled artisan will recognize that a large variety of other microtiter plates that are not the Immulon 1B 96-well plate allow for the immobilization of polypeptides (or antibodies) thereon, and therefore would be suitable for providing the solid support of the present disclosure.

An exemplary lateral flow device is the lateral flow device that is described in U.S. Pat. No. 5,726,010, which is incorporated herein by reference in its entirety. The device for performing a lateral flow assay may be a SNAP® device, which is commercially available from IDEXX Laboratories, Inc. of Westbrook, ME. However, it is to be understood that the skilled artisan will recognize that a large variety of other lateral flow devices that are not SNAPK devices or described by U.S. Pat. No. 5,726,010 allow for the immobilization of an polypeptide (or antibody) thereon, and therefore would be suitable for being used as the device of the present disclosure. These devices can include, for example, lateral flow devices that use colloidal gold technology.

The polypeptides or antibodies used in the device of the disclosure may be immobilized on the solid support by any methodology known in the art, including, for example, covalently or non-covalently, directly or indirectly, attaching the antibodies to the solid support. Therefore, while these antibodies or polypeptides may be attached to the solid support by physical adsorption (i.e., without the use of chemical linkers), it is also true that these antibodies or polypeptides may be immobilized to the solid support by any chemical binding (i.e., with the use of chemical linkers) method readily known to one of skill in the art.

It is also to be understood that the solid support may be any suitable material for the immobilization of the antibodies or polypeptides of the disclosure. For example, the solid support may be beads, particles, tubes, wells, probes, dipsticks, pipette tips, slides, fibers, membranes, papers, natural and modified celluloses, polyacrylamides, agaroses, glass, polypropylene, polyethylene, polystyrene, dextran, nylon, amylases, plastics, magnetite or any other suitable material readily known to one of skill in the art.

The device optionally may include one or more labeled antigen or antibody capture reagents that may be mixed with a sample from a mammal prior to application to a device of the disclosure. When the labeled capture antigen or antibody reagent is included, the labeled antigen or antibody capture reagent may or may not be deposited or dried on a solid surface of the device. “Antigen capture reagent” refers to any compound that is specific for the antigen or antigens of interest. The labeled antigen capture reagent, whether added to the mammalian sample or pre-deposited on the device, may be, for example, a labeled antibody specific for an A. abstrusus antigen, including, but not limited to, the antibodies of the present disclosure. In one example, anti-TDX1557 pAB conjugated with horseradish peroxidase may be used as a labeled antigen capture reagent. “Antibody capture reagent” refers to any compound that specifically binds to the antibody or antibodies of interest. The labeled antibody capture reagent, whether added to the mammalian sample or pre-deposited on the device, may be, for example, a labeled polypeptide specific for a A. abstrusus antibody, including, but not limited to, the polypeptides of the present disclosure. In one example, recombinant protein TDX1557 conjugated with horseradish peroxidase may be used as a labeled antibody capture reagent.

The device also may optionally include a liquid reagent that transports (such as when the device is a SNAP® device, for example), or otherwise facilitates removal of (such as when the device includes a microtiter plate, for example), unbound material (e.g., unreacted portions of the mammalian sample, such as, for example, unreacted portions of serum or fecal extract, and unbound antigen or antibody capture reagent) away from the reaction zone (solid phase). The liquid reagent may be a wash reagent and serve only to remove unbound material from the reaction zone, or it may include a detector reagent and serve to both remove unbound material and facilitate antigen or antibody detection. For example, in the case of an antigen or antibody capture reagent conjugated to an enzyme, the detector reagent includes a substrate that produces a detectable signal upon reaction with the enzyme-antibody conjugate at the reaction zone (solid phase). Alternatively, in the case of a labeled antigen or antibody capture reagent conjugated to a radioactive, fluorescent, or light-absorbing molecule, the liquid reagent acts merely as a wash solution facilitating detection of complex formation at the reactive zone by washing away unbound labeled reagent.

The liquid reagent may further include a limited quantity of an “inhibitor”, i.e., a substance that blocks the development of the detectable end product. A limited quantity is defined as being an amount of inhibitor sufficient to block end product development until most or all excess, unbound material is transported away from the second region, at which time detectable end product is produced.

The device of the present disclosure may also include various binding reagents immobilized at locations distinct from the antigen or antibody capture reagent or reagents. For example, an immunoreagent (an antibody, antigen or polypeptide) that recognizes a species-specific (e.g., A. abstrusus-specific) antibody portion of a labeled antibody or antigen capture reagent, or an enzyme portion of an enzyme-labeled reagent, can be included as a positive control to assess the viability of the reagents within the device. For example, a positive control may be an anti-horseradish peroxidase antibody that has been raised in, for example, goat or mouse. Additionally, a reagent, e.g., an antibody, isolated from a non-immune member of the species from which the antibody portion of the antigen-antibody complex was derived can be included as a negative control to assess the specificity of immunocomplex (i.e., antigen-antibody complex) formation.

In addition to being designed to detect A. abstrusus in a mammalian sample, the device of the disclosure optionally may be designed to allow one or more other diagnostic tests to be performed. For example, the solid support may also include reagents for the detection of one or more non-A. abstrusus lungworm parasites, one or more non-lungworm worm parasites, one or more non-worm parasites, one or more viruses, one or more fungi, or one or more bacteria. The reagents for the detection of one or more non-lungworm worm parasites, one or more non-worm parasites, one or more viruses, one or more fungi, or one or more bacteria may be, for example, one or more antibodies or one or more antigens recognized by antibodies specific for one or more non-lungworm worm parasites, one or more non-worm parasites, one or more viruses, one or more fungi, or one or more bacteria.

In one embodiment, the device of the present disclosure is a microtiter plate that includes a plurality of wells, wherein each well includes a solid support having a polypeptide of the disclosure, for instance, recombinant TDX1557, conservative variant of TDX1557, or truncated version of TDX1557, immobilized thereupon.

The plate may be used in conjunction with a method of the present disclosure to detect A. abstrusus lungworm in a mammalian sample. Specifically, a lungworm infection may be diagnosed in a mammal by detecting one or more lungworm antibodies with the polypeptide of the disclosure, for instance recombinant TDX1557, conservative variant of TDX1557, or truncated version of TDX1557 that is immobilized on the solid support. In one embodiment, the antibodies that are detected are lungworm antibodies. “Lungworm antibodies” are antibodies to any product or products of lungworm that are present in a sample, for instance a serum sample, and that can specifically and stably bind to the recombinant TDX1557, conservative variant of TDX1557, or truncated version of TDX1557. Lungworm antibodies therefore may be antibodies to whole lungworm, lungworm eggs, lungworm fragments, or products secreted, excreted or shed from lungworm or a combination thereof. Lungworm antigens may include any one of the polypeptides of the present disclosure, such as the polypeptides having an amino acid sequence corresponding to SEQ ID NO:2 through SEQ ID NO:17, polypeptides having an amino acid sequence that is a conservative variant of those sequences, and/or antigenic fragments of any such polypeptides, for example.

The disclosure further includes assay kits (e.g., articles of manufacture) for detecting A. abstrusus lungworm in a mammalian sample. A kit therefore may include one or more devices and/or compositions of the present disclosure. For example, the kit may include the polypeptides or antibodies of the disclosure and means for determining binding of the antigenic polypeptide of the disclosure to lungworm antibodies in a sample or the binding of the antibodies of the disclosure to lungworm antibodies in the sample. In one particular example, such a kit includes the device having an immobilized polypeptide of the disclosure, such as rTDX1557, for example, one or more antibody capture reagents (e.g., a non-immobilized labeled antibody capture reagent and an immobilized antibody capture reagent) and wash reagent, as well as detector reagent and positive and negative control reagents, if desired or appropriate. Other components such as buffers, controls, and the like, known to those of ordinary skill in art, may be included in such test kits. The relative amounts of the various reagents can be varied, to provide for concentrations in solution of the reagents that substantially optimize the sensitivity of the assay. Particularly, the reagents can be provided as dry powders, usually lyophilized, which on dissolution will provide for a reagent solution having the appropriate concentrations for combining with a sample. The present kit may further include instructions for carrying out one or more methods of the present disclosure, including instructions for using any device and/or composition of the present disclosure that is included with the kit.

### Methods of the Disclosure

The present disclosure further includes methods for using one or more of the devices, kits and/or compositions of the present disclosure to detect the presence or absence of A. abstrusus lungworm in a sample. The methods therefore may be carried out to detect the presence or absence of lungworm in a sample, such as, for example, a serum sample, that is obtained from a mammal, including, but not limited to, a canine, feline, porcine, bovine or human. Further, the methods may be carried out to detect roundworm, hookworm, whipworm and/or heartworm, for example. These methods further are useful for confirming such presence or absence of lungworm in a sample even when that sample includes one or more products derived from other worm species, including one or more products from hookworm, whipworm, roundworm, and/or heartworm.

In one embodiment of the methods of the present disclosure, detection of A. abstrusus lungworm may be accomplished by detecting the presence or absence of one or more lungworm antibodies in a sample using the polypeptides of the disclosure, such as the polypeptides having an amino acid sequence corresponding to SEQ ID NO:2 through SEQ ID NO:18, as well as fragments and/or conservative variants of those sequences, for example. In another embodiment of the disclosure, detection of lungworm may be accomplished by detecting the presence or absence of one or more lungworm antigens in a sample using the antibodies of the disclosure, such as rTDX1557 pAb, for example. The skilled artisan will recognize that there are a variety of ways of extracting and preparing non-fecal samples from a mammal as well. For example, the sample may be a serum sample obtained from blood or a sample obtained by swabbing the mammal, such as blood and related fluids such as serum and plasma, fluids and tissue obtained from pharyngeal swabs, transtracheal aspirates, tracheal swabs, bronchoalveolar lavage fluid, pleural effusions and other materials obtained from the respiratory tract of the mammal, for example. As yet another example, tissue sections, including tissue from small intestine, large intestine, cecum, colon, rectum, or another tissue of the gastrointestinal tract, may be obtained by biopsy.

One embodiment of the methods include contacting the mammalian sample with one or more polypeptides of the disclosure specific for one or more A. abstrusus lungworm antibodies under conditions that allow an antigen/antibody complex. i.e., an immunocomplex, to form. That is, an antigenic polypeptide that specifically binds to a lungworm antibody present in the sample. In another embodiment of the methods include contacting the mammalian sample with one or more antibodies of the disclosure specific for one or more lungworm antigens under conditions that allow an antigen/antibody complex. i.e., an immunocomplex, to form. That is, an antibody that specifically binds to a lungworm antigen present in the sample. The skilled artisan is familiar with assays and conditions that may be used to detect such antigen/antibody complex binding. For example, the antigen/antibody complex may be detected using a secondary antibody that binds to the antigen/antibody complex. The formation of a complex between a polypeptide of the disclosure and lungworm antibody in the sample may be detected using any suitable method known in the art.

Further, the relative amount of antibody-antigen complexes that are formed in one particular reaction may be measured with respect to those formed in any other reaction by any methodology known in the art for achieving that goal. When it is determined that a sample under test has more antibody-antigen complexes than does a control sample, it can be concluded that lungworm is present in the test sample. When this is true, it may be concluded that the mammal from which the test sample was obtained harbors an lungworm infection. Either one or both of the conclusions that lungworm is present in the test sample and that the mammal being tested harbors lungworm infection may be made by a clinician at a diagnostic service provider or by a caregiver of the mammal, such as the mammal's veterinarian, for example. When a caregiver of a mammal determines (or is otherwise informed that) a mammal harbors a lungworm infection, the caregiver may then subject the mammal to a course of treatment that is optimally designed to rid the mammal of lungworm specifically, rather than of a parasitic nematode infection generally. Further, the present disclosure can be used to confirm that any animal that has received treatment for lungworm infection has been rid of that infection.

The steps of the method of the present disclosure may include applying a mammalian sample to a device of the disclosure, which includes an immobilized polypeptide specific for one or more A. abstrusus lungworm antibodies (or an immobilized antibody specific for one or more lungworm antigens), and detecting the presence or absence of the lungworm antibodies (or antigens) in the sample. Polypeptides specific for antibodies of lungworm or antibodies specific for lungworm antigens may be directly or indirectly attached to a solid support or a substrate such as a microtiter well, polypeptide (or antibody)-immobilizing portion of a SNAP® device, magnetic bead, non-magnetic bead, column, matrix, membrane, fibrous mat composed of synthetic or natural fibers (e.g., glass or cellulose-based materials or thermoplastic polymers, such as, polyethylene, polypropylene, or polyester), sintered structure composed of particulate materials (e.g., glass or various thermoplastic polymers), or cast membrane film composed of nitrocellulose, nylon, polysulfone or the like (generally synthetic in nature). All of these substrate materials may be used in suitable shapes, such as films, sheets, or plates, or they may be coated onto or bonded or laminated to appropriate inert carriers, such as paper, glass, plastic films, or fabrics. Suitable methods for immobilizing peptides on solid phases include ionic, hydrophobic, covalent interactions and the like.

The methods of the present disclosure do not require the use of solid phases or substrates, however. The skilled artisan will recognize that there are a number of ways that the present method may be carried out to detect the presence or absence of A. abstrusus lungworm without involving the use of solid phases or substrates. In just one example, immunoprecipitation methods that do not require the use of solid phases or substrates may be carried out.

In some embodiments of the disclosure, the antigen/antibody complex is detected when an indicator reagent, such as an enzyme conjugate, which is bound to the antibody, catalyzes a detectable reaction. Optionally, an indicator reagent including a signal generating compound may be applied to the antigen/antibody complex under conditions that allow formation of a detectable antigen/antibody/indicator complex. Optionally, the antibody may be labeled with an indicator reagent prior to the formation of an antigen/antibody complex.

The formation of an antigen/antibody complex or an antigen/antibody/indicator complex in some of the methods of the present disclosure specifically may be detected by radiometric, colorimetric, fluorometric, photometric, size-separation, or precipitation methods. Detection of an antigen/antibody complex also may be accomplished by the addition of a secondary antibody that is coupled to an indicator reagent including a signal generating compound. Indicator reagents including signal generating compounds (labels) associated with a polypeptide/antibody complex may be detected using the methods described above and may include chromogenic agents, catalysts such as enzyme conjugates, fluorescent compounds such as fluorescein and rhodamine, chemiluminescent compounds such as dioxetanes, acridiniums, phenanthridiniums, ruthenium, and luminol, radioactive elements, direct visual labels, as well as cofactors, inhibitors, magnetic particles, and the like. Examples of enzyme conjugates include alkaline phosphatase, horseradish peroxidase, beta-galactosidase, and the like. The selection of a particular label is not critical, but it will be capable of producing a signal either by itself or in conjunction with one or more additional substances.

Methods of the disclosure include, but are not limited to those based on competition, direct reaction or sandwich-type assays, including, but not limited to ELISA, RIA, immuno-fluorescent assays (IFA), hemagglutination (HA), fluorescence polarization immunoassay (FPIA), and microtiter plate assays (i.e., any assay done in one or more wells of a microtiter plate). One assay of the disclosure includes a reversible flow chromatographic binding assay, which may be performed, for example, by using a SNAP, device. See U.S. Pat. No. 5,726,010.

In some embodiments, the method of the disclosure facilitates sandwich or competition-type specific binding assays. In a sandwich assay, antibody capture reagents are immobilized in a reactive zone. These antibody capture reagents may specifically bind to antibodies in the sample being tested for lungworm. Following binding of the antibody from the sample, the antibody capture reagent/antibody complex is detected by any suitable method. For example, the complex may be reacted with labeled specific binding reagents (e.g., an enzyme-antibody conjugate) and antibody detected (e.g., upon reaction with substrate).

In other embodiments of the method of the present disclosure, a competition assay is performed. In a competition assay, antibody capture reagents are immobilized at the reactive zone and are contacted simultaneously with antibody from a sample and labeled antibody (e.g., an antibody-enzyme conjugate). The amount of label detected at the reactive zone is inversely proportional to the amount of antibody in the sample.

In some embodiments of the method, polypeptides that bind specifically to A. abstrusus lungworm antibody or antibodies are attached to a solid phase or substrate. A sample potentially including an antibody from lungworm is added to the substrate. Secondary antibodies that specifically bind lungworm antibodies are added. These secondary antibodies may be linked to an indicator reagent, such as an enzyme conjugate. Wash steps may be performed prior to each addition. A chromophore or enzyme substrate may be added and color may be allowed to develop. The color reaction may be stopped and the color may be quantified using, for example, a spectrophotometer, and/or the color may be subjectively assessed by the human eye.

In other embodiments of the method, polypeptides specific for A. abstrusus lungworm antibodies or antibodies are attached to a solid phase or substrate. A sample potentially including a lungworm antibody is added to the substrate. Second anti-species antibodies that specifically bind antibodies of lungworms are added. These second antibodies are from a different species than are the solid phase antibodies. Third anti-species antibodies that specifically bind the second antibodies and that do not specifically bind the solid phase antibodies are added. The third antibodies may include an indicator reagent, such as an enzyme conjugate. Wash steps may be performed prior to each addition. A chromophore or enzyme substrate may added and color may be allowed to develop. The color reaction may be stopped and the color may be quantified using, for example, a spectrophotometer, and/or the color may be subjectively assessed by the human eye.

In a specific example, the method of the present disclosure is performed in conjunction with a device that is a lateral flow assay device by adding a prepared mammalian sample to a flow matrix of the device at a first region (a sample application zone). The prepared sample is carried in a fluid flow path by capillary action to a second region of the flow matrix where a particulate label capable of binding and forming a first complex with an antibody in the sample exists. The particulate label can be, e.g., a colored latex particle, dye sol, or gold sol conjugated to an antibody specific for A. abstrusus lungworm antibody. The first complex is carried to a third region of the flow matrix where a polypeptide that specifically binds to the lungworm antibody is immobilized at a distinct location. A second complex is formed between the immobilized polypeptide and the first complex. The particulate label that is part of the second complex can be directly visualized by the human eye.

The polypeptide may be an immobilized antibody capture reagent in a reaction zone (solid phase). A second antibody capture reagent, i.e., a second antibody that has been conjugated to a label, either may be added to the sample before the sample is added to the device, or the second antibody capture reagent can be incorporated into the device. For example, the labeled antibody capture reagent may be deposited and dried on a fluid flow path that provides fluid communication between a sample application zone and the solid phase. Contact of the labeled antibody capture reagent with the test sample can result in dissolution of the labeled antibody capture reagent.

In other embodiments of the method, antibodies that bind specifically to A. abstrusus lungworm antigen or antigens are attached to a solid phase or substrate. A sample potentially including antigen or antigens from lungworm is added to the substrate. Secondary antibodies that specifically bind lungworm antigens are added. These secondary antibodies may be linked to an indicator reagent, such as an enzyme conjugate. Wash steps may be performed prior to each addition. A chromophore or enzyme substrate may be added and color may be allowed to develop. The color reaction may be stopped and the color may be quantified using, for example, a spectrophotometer, and/or the color may be subjectively assessed by the human eye.

In other embodiments of the method, antibodies specific for A. abstrusus lungworm antigen or antigens are attached to a solid phase or substrate. A sample potentially including a lungworm antigen is added to the substrate. Second anti-species antibodies that specifically bind antigen of lungworms are added. These second antibodies are from a different species than are the solid phase antibodies. Third anti-species antibodies that specifically bind the second antibodies and that do not specifically bind the solid phase antibodies are added. The third antibodies may include an indicator reagent, such as an enzyme conjugate. Wash steps may be performed prior to each addition. A chromophore or enzyme substrate may added and color may be allowed to develop. The color reaction may b^{e} stopped and the color may be quantified using, for example, a spectrophotometer, and/or the color may be subjectively assessed by the human eye.

In a specific example, the method of the present disclosure is performed in conjunction with a device that is a lateral flow assay device by adding a prepared mammalian sample to a flow matrix of the device at a first region (a sample application zone). The prepared sample is carried in a fluid flow path by capillary action to a second region of the flow matrix where a particulate label capable of binding and forming a first complex with an antigen in the sample exists. The particulate label can be, e.g., a colored latex particle, dye sol, or gold sol conjugated to an antibody specific for a lungworm antigen. The first complex is carried to a third region of the flow matrix where a antibody that specifically binds to the lungworm antigen is immobilized at a distinct location. A second complex is formed between the immobilized antibody and the first complex. The particulate label that is part of the second complex can be directly visualized by the human eye.

The antibody may be an immobilized antigen capture reagent in a reaction zone (solid phase). A second antigen capture reagent, i.e., a second antibody that has been conjugated to a label, either may be added to the sample before the sample is added to the device, or the second antigen capture reagent can be incorporated into the device. For example, the labeled antigen capture reagent may be deposited and dried on a fluid flow path that provides fluid communication between a sample application zone and the solid phase. Contact of the labeled antigen capture reagent with the test sample can result in dissolution of the labeled antigen capture reagent.

In one embodiment of the methods of the present disclosure, A. abstrusus lungworm antibody (or A. abstrusus lungworm antigen) is detected by ELISA. Specific examples of the ELISA method of the present disclosure is described in the Example section included herein. Although the present disclosure is described with respect to those specific ELISA methods, however, it is to be understood that those of ordinary skill in the art will recognize that alternative, additional or substitute ELISA steps may be used without deviating from the basic goal achieved through this method of the disclosure.

In another embodiment of the methods of the present disclosure, A. abstrusus lungworm antibody (or A. abstrusus lungworm antigen) is detected by using a lateral flow device, such as a SNAP® device, for example.

Further, the methods of the disclosure for detection of lungworm infection can be combined with other diagnostic assays to detect the presence of other organisms or conditions. For example, assays of the disclosure can be combined with reagents that detect one or more non-lungworm worm fecal parasites, one or more non-worm fecal parasites, one or more viruses, one or more fungi, one or more bacteria, one or more blood-borne parasites or occult blood or a combination thereof. By providing two or more unique binding sites in a single assay device (such as, for example, two unique spots on a SNAP® assay device), the present disclosure allows for detection of two or more organisms from a single sample. In one embodiment, there are three unique spots for detection of past or present infection or infestation from three organisms (the spots being either antigen or antibody binding reagents) from a single sample (i.e., the same individual sample is exposed to the three capture reagents on a single device). In yet another embodiment, there are four unique spots for detection of past or present infection or infestation from four organisms (the spots being either antigen or antibody binding reagents) from a single sample (i.e., the same individual sample is exposed to the four capture reagents on a single device. It is to be understood, however, that the same device may include more than four unique spots and/or allow for the detection of more than four organisms.

The reagents for the detection of one or more non-lungworm worm parasites, one or more non-worm parasites, one or more viruses, one or more fungi, or one or more bacteria may be, for example, one or more antibodies or one or more antigens recognized by antibodies specific for one or more non-lungworm worm parasites, one or more non-worm parasites, one or more viruses, one or more fungi, or one or more bacteria.

When a device of the present disclosure includes reagents for the specific detection of hookworm, reagents for the specific detection of lungworm, and reagents for the specific detection of roundworm, for example, in addition to the reagents for detecting lungworm, the method of the present disclosure may involve using that device for the additional purpose or purposes of determining whether the sample that is being tested for lungworm also includes hookworm, whipworm, roundworm and/or heartworm. In this arrangement, therefore, the method/device of the present disclosure would not only be able to specifically confirm that lungworm is present in or absent from any particular test sample, but it would also be useful for specifically confirming that the sample includes or does not include any antigen of hookworm, any antigen from whipworm and/or any antigen of roundworm. The capability to specifically detect lungworm and one or more other organisms by applying a single sample to the device of the disclosure would be useful to the caregiver of the animal from which the sample under test was obtained. A caregiver who learns that a sample includes both lungworm and roundworm, but not whipworm or hookworm, for example, could use that knowledge to treat the mammal from which the sample was taken specifically for lungworm by administering to that mammal a drug optimally effective against lungworm and a second drug optimally effective against roundworm. Absent such knowledge, the caregiver may, for example, otherwise treat the mammal with a drug that is optimally effective against only lungworm, only roundworm, or neither lungworm nor roundworm (in such cases, the mammal would be at risk of receiving suboptimal treatment). In addition, humans who may come in contact with the infested animal or its excretions may be advised to take precautions against acquiring the parasite or parasites. In this context, it is important to determine the worm species with high specificity, as some helminths, such as roundworms and hookworms, can cause significant disease (i.e., larva migrans, severe enteritis or allergic reactions) in humans, while it is generally accepted that lungworm does not play a zoonotic role of importance in humans.

The method further may optionally include using one or more nucleic acids from lungworm, including, but not limited to, the nucleic acids of the present disclosure, to determine the presence or absence of lungworm in a mammalian sample. Such use of these nucleic acids for determining the presence of lungworm may be carried out before, after or concomitantly with the carrying out of any other aspects of the method, including the detection of lungworm by antibody. Therefore, in one aspect, after lungworm is detected or not detected in a particular sample and the mammal from which the sample was obtained is diagnosed as either having or not having a lungworm infection, the sample (or a later-obtained sample from the diagnosed mammal) may be tested for the presence or absence of any one or more of the nucleic acids, including any one or more nucleic acids of the disclosure. Anyone failing to detect lungworm in a particular mammal by using one or more nucleic acids (after the lungworm had been detected by using one or more antibodies) would need to take into consideration the possibility that the antibodies had detected lungworm antigen prior to the appearance of detectable lungworm nucleic acid in the sample. In such an instance, the mammal's caregiver may elect to ignore the observation that the nucleic acid had failed to detect the lungworm and proceed with treating the mammal specifically for lungworm infection based on the observation that the antibodies had in fact detected lungworm. In another aspect, the nucleic acids are used to determine the presence or absence of lungworm in a particular mammal, and then the presence or absence of lungworm is further evaluated by using the antibodies of the present disclosure. Detection of one or more lungworm nucleic acids may be carried out by using any nucleic acid detection techniques known to the skilled artisan. For example, such detection may be carried out by performing a PCR-based technique, such as, but limited to, for example, a real-time PCR-based technique. Exemplary PCR-based techniques are described in, e.g., PCR Protocols (Methods in Molecular Biology), 2^{nd} ed., Bartlett and Stirling, eds., Humana Press (2003); and Sambrook and Russell, Molecular Cloning: A Laboratory Manual, Cold Spring Harbor Laboratory Press (2001); each one of which is incorporated herein by reference in its entirety.

### Vaccines and Pharmaceutical Compositions

In another aspect of this disclosure, vaccines and pharmaceutical compositions are provided for the prevention of A. abstrusus infection, and complications thereof, in a mammal. The pharmaceutical compositions generally comprise one or more polypeptides, containing one or more epitopes of A. abstrusus proteins, or comprise one or more polynucleotides encoding one or more of the polypeptides and a physiologically acceptable carrier. The vaccines comprise one or more of the above polypeptides or polynucleotides and an adjuvant, for enhancement of the immune response.

The polypeptides defined above, and also the polynucleotides encoding these polypeptides, can be used for preparing immunogenic compositions, and in particular anti-A abstrusus vaccines.

The immunogenic composition comprising one or more polypeptide(s) as defined above, or one or more polynucleotide(s) encoding said polypeptide(s), can be combined with one or more adjuvant(s) for enhancing the immune response. In some embodiments, the composition may comprise one or more other immunogenic polypeptide(s) recognized by anti-A. abstrusus antibodies.

In some embodiments, the immunogenic composition in accordance with the present disclosure is a vaccine.

Where appropriate, in particular in the case of short peptides (530 amino acids), said polypeptide(s) can be coupled to a carrier protein. By way of examples of carrier proteins, mention will in particular be made of KLH (keyhole limpet hemocyanin), bovine serum albumin (BSA), ovalbumin, tetanus toxoid or diphtheria toxoid. It is also possible to form a multiepitope composition, by combining several copies of the same peptide with one another, and optionally with other peptide epitopes, in the form of chimeric polypeptides, or by means of a polymeric chain, for example a polylysine.

If a polynucleotide is used as immunogen, the immunogenic composition may be in the form of a recombinant vector into which the polynucleotide(s) to be administered is (are) inserted. Use may be made, for example, of the viral vectors such as poxviruses, adenoviruses, retroviruses, lentiviruses, herpesviruses and AAVs (adeno-associated viruses), etc. It can also be in the form of a nonpathogenic bacterium transformed with one or more expression vectors containing said polynucleotide(s). It is also possible to administer the polynucleotide(s) directly, in the form of naked DNA, or to incorporate it (them) into liposomes. In the case of a vaccine, a non-pathogenic bacterium (for example a lactobacillus, or a nonpathogenic strain of Escherichia coli or Salmonella suis), or a vector derived from a vaccine viral strain may be useful.

In one embodiment, polynucleotides of the present disclosure can be administered to a mammal in a fashion to enable expression of that molecule into a protective protein (e.g., mRNA) or protective RNA (e.g., antisense RNA, ribozyme or RNA drug) in the mammal to be protected from disease. Nucleic acid molecules can be delivered to an mammal in a variety of methods including, but not limited to, (a) direct injection (e.g., as “naked” DNA or RNA molecules, such as is taught, for example in Wolff et al., 1990, Science 247, 1465-1468), (b) packaged as a recombinant virus particle vaccine or as a recombinant cell vaccine (i.e., delivered to a cell by a vehicle selected from the group consisting of a recombinant virus particle vaccine and a recombinant cell vaccine), or (c) encapsulated in liposomes or lipid nanoparticles. In some embodiments, the polynucleotides can include one or more modified uridine residues such as pseudouridine or methylpseudouridine.

A recombinant virus particle vaccine of the present disclosure includes a recombinant molecule of the present disclosure that is packaged in a viral coat and that can be expressed in an animal after administration. Preferably, the recombinant molecule is packaging-deficient. A number of recombinant virus particles can be used, including, but not limited to, those based on alphaviruses, poxviruses, adenoviruses, herpesviruses, and retroviruses. Methods to produce and use recombinant virus particle vaccines are known in the art.

Any of a variety of adjuvants may be employed in the immunogenic compositions of this disclosure to nonspecifically enhance the immune response, for instance, by increasing the immunogenicity of the polypeptides. Most adjuvants contain a substance designed to protect the antigen from rapid catabolism, such as alum (aluminum hydroxide) or mineral oil, and a nonspecific stimulator of immune response, such as lipid A, Bordella pertussis or Mycobacterium tuberculosis. Such adjuvants are commercially available as, for example, Freund's Incomplete Adjuvant and Complete Adjuvant (Difco Laboratories, Detroit, Mich.) and Merck Adjuvant 65 (Merck and Company, Inc., Rahway, N.J.), liposomes, virosomes (reconstituted viral envelopes), peptide derivatives of muramic acid, etc. In the case of a vaccine a pharmacologically acceptable adjuvant will of course be chosen; by way of examples of adjuvants, mention will be made of adjuvants of “oil-in-water” emulsion type.

While any suitable carrier known to those of ordinary skill in the art may be employed in the pharmaceutical compositions of this disclosure, the type of carrier will vary depending on the mode of administration. For parenteral administration, such as subcutaneous injection, the carrier preferably comprises water, saline, alcohol, a fat, a wax or a buffer. For oral administration, any of the above carriers or a solid carrier, such as mannitol, lactose, starch, magnesium stearate, sodium saccharine, talcum, cellulose, glucose, sucrose, and magnesium carbonate, may be employed. Biodegradable microspheres (e.g., polylactic galactide) may also be employed as carriers for the pharmaceutical compositions of this disclosure.

Routes and frequency of administration and polypeptide or polynucleotide doses will vary from individual to individual and may parallel those currently being used in immunization against other protozoan infections. In general, the pharmaceutical compositions and vaccines may be administered by injection (e.g., intramuscular, intravenous or subcutaneous), intranasally (e.g., by aspiration) or orally. Between 1 and 4 doses may be administered for a 2-6 week period. In one embodiment, two doses are administered, with the second dose 2-4 weeks later than the first. A suitable dose is an amount of polypeptide that is effective to raise antibodies in a treated mammal that are sufficient to protect the mammal from A. abstrusus infection for a period of time. In general, the amount of polypeptide present in a dose ranges from about 1 μg to about 100 mg per kg of host, typically from about 10 μg to about 1 mg, and preferably from about 100 μg to about 1 μg. Suitable dose sizes will vary with the size of the animal, but will typically range from about 0.01 mL to about 5 mL for 10-60 kg animal.

The present disclosure is specifically described with reference to certain specific Examples; however, it is not to be construed as being limited thereto.

### EXAMPLES

Unless otherwise indicated, the following materials and techniques were used to generate data described in one or more of Examples 1-12 as described below.

Feline lungworm extract preparation. A. abstrusus worms were obtained from Dr. Bowman, Cornell University, Ithaca, NY. The whole worms were washed several times with cold PBS, pH 7.0, to remove any fecal materials and mucus form the hosts in room temperature and homogenized at 4° C. with a sonicator until no obvious tissue chunks were visible to the naked eye. The homogenized materials were centrifuged at 10,000 g for 30 minutes at 4° C. and the supernatant was carefully removed. Protein concentration was determined using Bradford assay.

Heartworm extract preparation. Heart worms [Dirofilaria immitis] were obtained from IDEXX Laboratories, Westbrook, ME. The whole worms were disrupted and suspended in cold PBS, pH 7.0 and homogenized at 4° C. with a polytron homogenizer. The homogenized materials were centrifuged at 10,000 g for 30 minutes at 4° C. and the supernatant was carefully removed and used directly in Experiment 3. Protein concentration was determined using Bradford assay.

Roundworm extract preparation. Round worms [Toxocara spp] were obtained from Antibody systems Inc, Hurst, TX and was processed as described in U.S. Pat. No. 7,951,547, which is incorporated by reference in its entirety. The whole worms were disrupted and suspended in cold PBS, pH 7.0 and homogenized at 4° C. with a polytron homogenizer [Kinematica, Bohemia, NY]. The homogenized materials were centrifuged at 10,000 g for 30 minutes at 4° C. and the supernatant was carefully removed and used directly in Experiment 4. Protein concentration was determined using Bradford assay.

Larval shedding procedure: For detection of feline lungworm, a visual fecal Baermann test is typically employed for the detection of parasite larvae rather than eggs in stool. See Baermann method as described in A J Carruth et al., Journal of Feline Medicine and Surgery 2019, Vol. S. pp. 1-6)

Feline fecal sample preparation. Feline animals known to be free of lungworm infection or be infected with lungworm provided the source of fecal samples. Samples (approximately 1 gram) from frozen, unpreserved feline fecal samples were suspended in 4 ml of diluent solution (“diluent solution” is 0.05 M Tris base; 1 mM EDTA; 0.45% Kathon; 16 mg/l gentamicin sulfate; 0.05% Tween-20; 40% fetal bovine serum; 10% rabbit serum; and 5% mouse serum). The suspension was centrifuged in a tabletop centrifuge at 4000 rpm for 20 minutes to produce a first supernatant. The first supernatant was centrifuged at 10,000 g for 5 minutes to produce a second supernatant, which is referred to herein as “fecal extract”.

Feline serum sample preparation. Feline animals known to be free of lungworm infection or be infected with lungworm provided the source of serum samples. Serum samples from six experimentally infected cats were obtained from Dr. Bowman's laboratory at Cornell University and were used throughout this study. Cats were infected at approximately 12-14 weeks of age. Each cat received 250 third-stage larvae (L3s) harvested from the lab's colony of Biomphalaria glabrata aquatic snails to commence their infection. Weekly fecal samples were collected and a Baermann procedure was performed to calculate a larval shedding rate in larvae per gram for each cat. Weekly blood draws were also performed with samples taken from altemating medial saphenous veins. The experimental infection continued until day 89, at which point Imidacloprid and Moxidectin therapy (Advantage Multi, Bayer) was introduced. The cats received at least two doses of this over the course of two months. Weekly fecal and blood draws were still performed until day 189.

Recombinant TDX1557 protein preparation. A codon optimized version (SEQ ID NO:19) of the 2M2 gene was synthesized and cloned into pCDNA 3.1 (TDX1557).

(SEQ ID NO: 19)
ATGGACTGGACTTGGAGAGTGTTCTTCCTGCTGGCCCTGGCTACAGGCG
TGCACAGCGAGAACAACGACGAGATGGACGAGAACAATCCCGTGGCTCT
GCCCGGCAAGAGCAATGTGTCTGAGCCTTGCCAGACCACCACCAGCAAT
CCTATTCACCCTACCACCGGCGAGCAGCTGGAAACCACAACAGGCAAGC
CCAGACGGACCACCAAGGGCAGAATCCTGAAAACCACCACCGTGAAGCC
CATCAAGACAACAAAGGGCAAGAAGCTCAAGACGACCAAGGGGAAGCTG
CTGAAAACAACAACAGTCAAACCCTTCAAGACCACGAAGGGAAAGCCCC
TTAAGACAACCACCAAAGAGCCCTTCCAGAGCACCACAGGTGGACCAGT
GGGCCCAACCACCGATAGATGCAAGCTGAACAACGGCATGAACGACCAG
CTGCGGGACATCATGCTGAACGATCACAACACCCTGAGAAGCCTGGCCG
CCAAAGGACTGGCCGAAAATCCTCTGGGCACCAACGGCAGAGCCCCTAA
AGCTGCCAGAATGCTGAAGATGGTGTACGACTGCAACGTGGAAAAGACC
GCCATGATCCACGCCAAGAAATGCGTGTTCGAGCACAGCAAAGGCCGGA
AGGATACCGGCGAGAACGTGTGGGTTATGTGGCCCGCTCAGAAAAACCT
GACCGAGATGGCCACCAGAGCCAGCAAGAGTTGGTTCGACGAGCTGAAG
AAATTCGGCGTGCCACCTGACAACATCCTGACCGAAGGCCTGTGGAACA
GACCCAAGATGGCCATTGGCCACTACACCCAGATGGTCTGGGAGGGCAG
CTACAAGCTTGGATGTGGCGTGGCCACATGCAGCGACAAGACCCTGATC
GTGTGCCAGTATAGCCCTGCCGGCAACTACATCGGCAGCATCATCTATG
CCATCGGCGAGCCCTGCAAGACCGACGAGGATTGCAAGTGCGAGGGCTG
CAAGTGCAGCAGAGATGAGGCCCTGTGCATCAAGCCCAATCACCACCAT
CATCACCATCACGGCGTGCTGGCCCCTCACGATTCTGTTCTTCAGTAAT
AA

Sequences encoding the HIS tag and an ADX18 epitope tag were added to the construct (SEQ ID NO: 20) (See FIG. 1B) at the C-terminal region for the purification of recombinant TDX1557.

While making the construct (SEQ ID NO:20), a sequence encoding an IgG-like signal sequence

(SEQ ID NO: 21)
MDWTWRVFFLLALATGVHSEN

was included in positions 941 to 1003 of SEQ ID NO:2) at the amino-terminal region of 2M2 to secrete the recombinant 2M2 (rTDX1557) polypeptide (SEQ ID NO:3) into the supernatant. The C-terminal region is tagged with tags (AAAHHHHHHHGVLAPHDSVLQ) (SEQ ID NO:22) to facilitate the purification of recombinant proteins. The recombinant construct was transfected into a mammalian expression system (HEK293 cells) using ExpiFectamine™ transfection kit (Thermo Fisher Scientific, Carlsbad, CA). The supernatant was collected after 5-7 days and the recombinant protein TCX1557 (SEQ ID NO:3) was purified using immobilized metal affinity chromatography (IMAC).

(SEQ ID NO: 3)
MDWTWRVFFLLALATGVHSENNDEMDENNPVALPGKSNVSEPCQTTTSNP
IHPTTGELETTTGKPRRTTKGRILKTTTVKPIKTTKGKKLKTTKGKLLKT
TTVKPFKTTKGKPLKTTTKEPFQSTTGGPVGPTTDRCKLNNGMNDQLRDI
MLNDHNTLRSLAAKGLAENPLGTNGRAPKAARMLKMVYDCNVEKTAMIHA
KKCVFEHSKGRKDTGENVWVMWPAQKNLTEMATRASKSWFDELKKFGVPP
DNILTEGLWNRPKMAIGHYTQMVWEGSYKLGCGVATCSDKTLIVCQYSPA
GNYIGSIIYAIGEPCKTDEDCKCEGCKCSRDEALCIKPNHHHHHHGVLAP
HDSVLQ

Polyclonal feline lungworm antibody preparation. The polyclonal antibodies “anti-TDX1557 pAB,” (IgG) was raised in rabbit against a polypeptide having amino acid sequence corresponding to SEQ ID NO:3, respectively and purified from serum by using standard methods. After rTDX1557 was introduced into rabbits, anti-TDX1557 pAB was purified from the plasma of the immunized rabbits by isolating IgG antibody by protein G affinity chromatography.

Infection of feline animals. Parasitic lungworm infection was effected by orally administering about 150-300 [infective larvae of A. abstrusus to a healthy feline. Infection was confirmed by microscopic observation of worm ova in fecal samples obtained from these host animals. Larval shedding was performed using standard Baermann method (A. J. Carruth et al. Journal of Feline Medicine and Surgery, 2019, Vol. 5, pp. 1-6). The infected felines were treated with anti-parasitic drug Imidacloprid and Moxidectin at Day 90.

ELISA assays. Lungworm lysate or recombinant TDX1557 (“rTDX1557”) (100 μl/well; 3 μg/ml for rTDX1557 and ˜2 ug/mL lysate) were immobilized by physical adsorption on Immulon IV 96-well plates overnight at 4° C. The plates were then blocked with 1% BSA in 0.1M Tris pH 7.0 for 2 hours at room temperature. The wells were then washed five times with a PBS-Tween-20 solution according to standard methods known to those of ordinary skill in the art. Serum samples from experimentally infected felines were diluted with PRRS diluent (IDEXX PRRS X3 Ab Test, Cat. No. 99-18070, IDEXX Laboratories, Inc., Westbrook, ME, USA) at 1 to 200 dilution (100 μl/well; (I to 200 dilution) and added to the wells. Following an hour incubation period at room temperature, the wells were washed five times using PBS-Tween-20 solution according to standard methods known to those of ordinary skill in the art. 50 μl of 1 to 5000 diluted Goat anti-feline IgG(H+L)-HRP antibody (Jackson ImmunoResearch Inc., West Grove, PA) in Enzyme conjugate diluent (IDEXX Laboratories, Westbrook, ME) was then added to each well and the plates were incubated for 30 minutes at room temperature. The plates were washed and 50 μl of 3,3′,5,5′-Tetramethylbenzidine (TMB) substrate (IDEXX Laboratories, Westbrook, ME) was added to each well and the plates were incubated for 15 minutes at room temperature. After stopping each enzymatic reaction with malic acid, the optical density (OD) value of each well of the 96-well plate was measured at A450 by standard spectrophotometric techniques by using an ELISA plate reader to generate an “OD450 value” (or, more simply, an “OD value”) for each well. In this arrangement, the OD value obtained for any particular well of the 96-well plate was directly proportional to the amount of specifically bound antibody present in the well.

Immunoblot procedure: The worm lysates (3 ug/ml) or recombinant TDX1557 (10 ug) were mixed with SDS-PAGE sample buffer and ran on SDS-PAGE gel electrophoresis for 35 minutes at 200V (Thermo Fisher, Carlsbad. MA). The total proteins of the lysate from SDS-PAGE gel was transferred to nitrocellulose (NC) membrane using iblot (Thermo Fisher, Carlsbad, CA). The NC membrane was probed with rabbit polyclonal antibodies against TDX1557, followed by the addition of Anti rabbit IgG H+L coupled with alkaline phosphatase (Sigma, St Louis, MO)). The blot was developed with BCIP/NBT substrate (Sigma, St Louis, MO).

ELISA procedure using Neutravidin plate for peptide array analysis. Neutravidin plates (Thermo Fisher, Carlsbad, CA) were coated with 100 ul of PBS diluted peptides in Neutravidin plate and the plates were incubated for 1 hour at RT, shaking. Afterwards, the plates were washed 3× with PBS-T. Dilute feline serum sample 1:800 in 1×PBS (pH 7.2+2% BSA) and apply 100 ul (what is concentration?) per well and incubate the plates for 1 hour at RT, shaking. Afterwards, the plates are washed 3× with PBS-T. 100 ul of secondary antibody dilution was the added to each well followed by 12 ul goat anti-cat H+L HRP in 60 mls ECD. The plates are then incubated for 1 hour at RT, shaking, washed 5× with PBS-T, then developed with 100 ul TMB substrate. The stop solution was added, and the solution was read at 450 nm.

### Example 1: Preparation and Characterization of Recombinant TDX1557

In this example, a recombinant TDX1557 (also referred to recombinant 2M2 or as rTDX1557) (SEQ ID NO:3) was expressed in a transfected HEK293 mammalian cell line and purified as discussed above. The purified protein was analyzed with SDS-PAGE Electrophoresis. The predicted molecular weight of 2M2 (TDX1557) is 28 kDa, but the recombinant TDX1557 had a molecular weight of 62 KDa due to post translation modification in the mammalian expression system. To further confirm that the recombinant TDX protein was produced, an immunoblot was performed as described by the procedure above. The gel was blotted to nitrocellulose membrane and probed with alkaline phosphatase tagged monoclonal antibody (Sigma, St Louis, MO) against HIS tag. The substrate NBT/BCIP (Sigma, St Louis, MO) was added for blot development as described in the procedure above. The gel and immunoblot results are shown in FIGS. 1A-1C.

As shown in FIG. 1A, an SDS-PAGE of analysis of supernatant from mammalian expression system confirmed expression of recombinant 2M2. In FIG. 1B, an immunoblot analysis of recombinant 2M2 is shown. The recombinant 2M2 was probed with mAb HIS tagged with Alkaline Phosphatase followed by development with BCIP/NBT as described above.

### Example 2: Detection of Lungworm Infection Using ELISA Assays Using TDX1557

This example compares detection and measurement of lungworm infection using a fecal Baermann technique and indirect ELISA using lungworm lysate and rTDX1557.

Experimental infection of Cats and procedures: Cats were orally infected with A. abstrusus larvae as described above. The fecal samples and blood draw were collected over a defined time period (Day 0, 7, 14, 21, 28, 35, 42, 49, 56, 63, 70, 77, 84, 91, 98, 112, 126, 140, 154, 168 and 182). The infected felines were treated with anti-parasitic drug Imidacloprid and Moxidectin by Day 90. Baermann method for visual detection and for counting lungworm larvae was performed on the fecal samples using standard procedures (A J Carruth et al., JFMS, 2019, Vol. 5, pp. 1-6). The serum samples collected during the defined time period were evaluated in an indirect ELISA assay with lungworm lysate and rTDX1557. A. abstrusus lysate was prepared from lungworm larvae as described above. The total protein in the lysate was determined by conventional Bradford assay. The recombinant protein TDX1557 was obtained as describe above.

ELISA assays for Aelurostrongylus abstrusus lysate and rTDX1557.

The lungworm larvae shedding rate was determined by standard Baermann method using fecal samples from experimentally infected samples. Indirect ELISA assays for lungworm lysate and rTDX1557 were performed using the procedure described above. Two felines (BigMac and Burrito) were experimentally infected with lungworm and serum samples from these felines were diluted with PRRS buffer (IDEXX PRRS X3 Ab Test, Cat. No. 99-18070, IDEXX Laboratories, Inc., Westbrook, ME, USA) and were used for the ELISA procedure.

The lungworm lysate or rTDX1557 were coated on an Immulon IV plate for an overnight period at 4 C, and the plates were blocked with blocking solution (1% BSA in 100 mM Tris pH 7.5). The plates were washed with 5×PBST, and the serum samples from experimentally infected cats (BigMAC or Burrito) were diluted with PRRS diluent and added to the plates, and kept it for an hour at RT. The plates were washed again with 5×PBST, and the secondary antibody (Anti-Cat IgG H+L) was added to the plates and kept it for 30 minutes. The plates were washed, and the substrate was added and read at 450 nm.

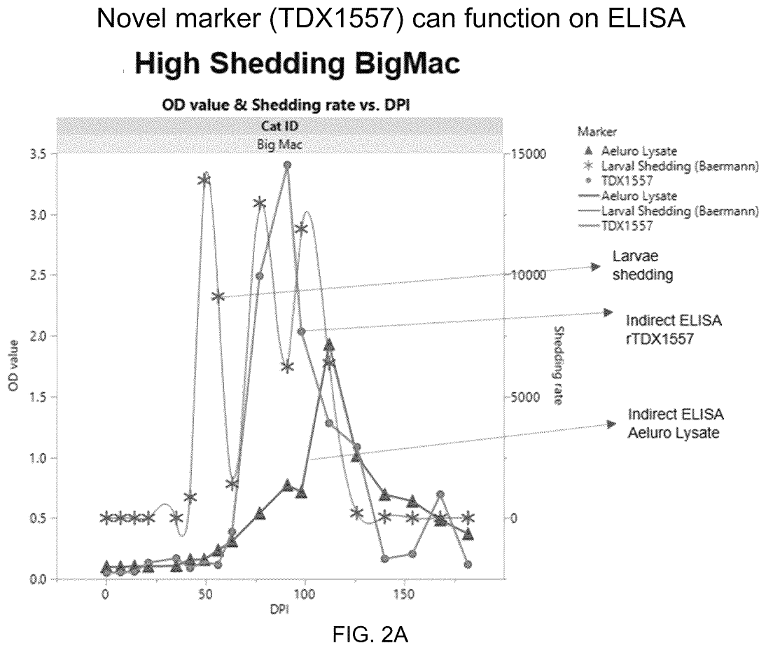

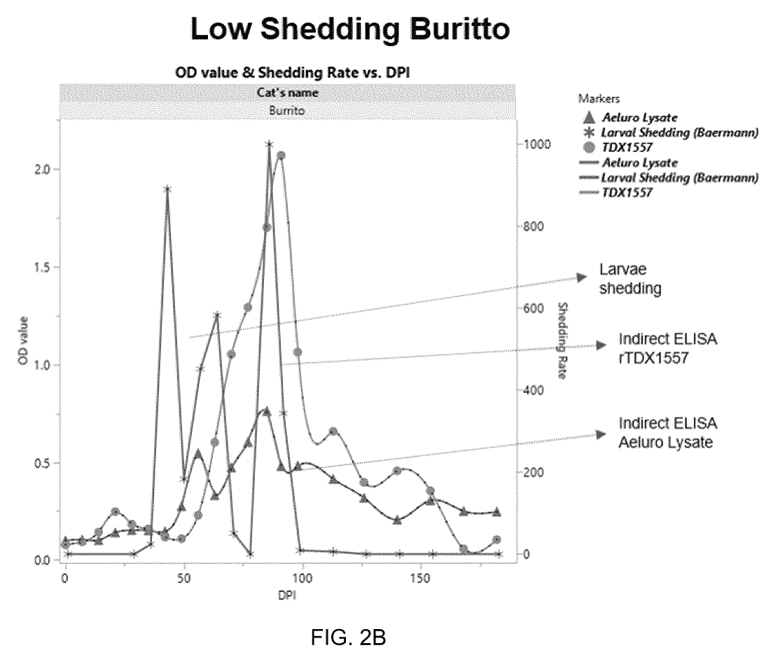

The larval shedding results for the experimentally infected feline are shown in FIGS. 2A and 2B, where the larvae shedding rate was measured over a period of 182 days. ELISA assays were performed with A. abstrusus lysate and rTDX1557 (2M2) to compare the larval shedding rate in a high shedding cat (“BigMac”) (see FIG. 2A), with the larval shedding in a low shedding cat (“Burrito”) (see FIG. 2B). The X-axis indicates days after post-infection (DPI). Y1 & Y2 axis represent OD Value and reflect the shedding rate, respectively. The results indicate that larval shedding measured by fecal Baermann methods produced inconsistent results, but the ELISA assays with either lungworm lysate and rTDX1557 show a gradual increase in antibody titer against lungworm but the antibody titer falls after day 90, indicating that the treated cats responded to treatment. ELISA assay with recombinant TDX1557 shows a much more robust antibody response compared to ELISA assay with lungworm lysate.

### Example 3: No Cross-Reactivity of Anti-TDX1557 with Heartworm

This example illustrates that rabbit polyclonal antibodies raised against rTDX1557 did not cross-react with heartworm (Dirofilaria immitis) using SDS-PAGE and immunoblot analysis.

SDS-PAGE and Immunoblot Analysis of A. abstrusus and Heartworm Lysates.

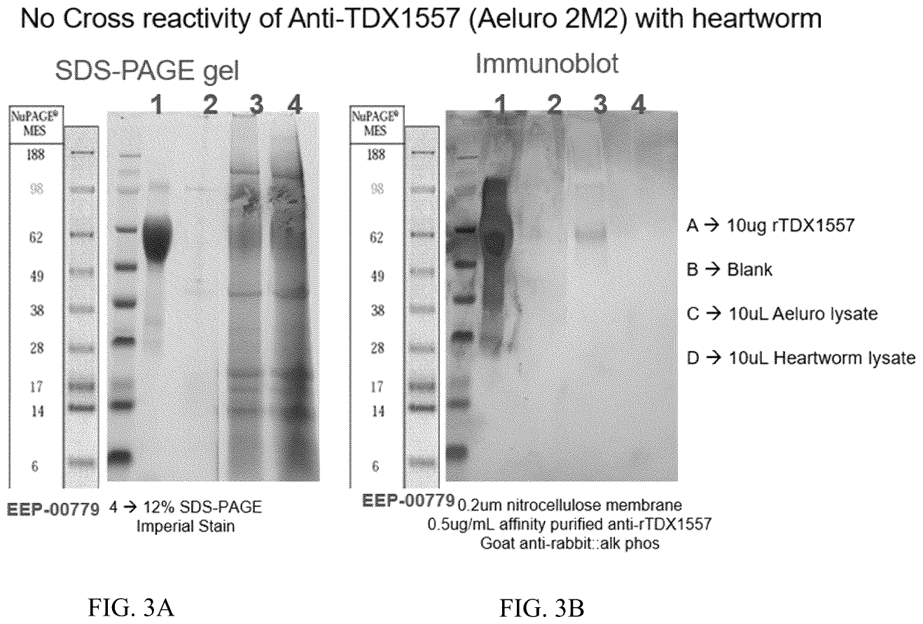

Rabbit polyclonal antibodies against rTDX1557, heartworm lysate, and lungworm lysates were prepared as described above. The rTDX1557, lungworm lysates and heartworm lysates were individually mixed with SDS-PAGE buffer and ran on SDS-PAGE gel electrophoresis in two different gels using conventional procedures. In the first gel, rTDX1557 (lane 1), A. abstrusus lungworm lysate (lane 3) and heartworm lysate (lane 4) were stained with imperial staining. The second gel, with identical lanes, was subjected to immunoblot analysis by transferring the total proteins of the lysates to nitrocellulose (NC) membrane. The NC membrane was probed with rabbit polyclonal antibodies against TDX1557, followed by the addition of alkaline phosphatase conjugates (Anti rabbit IgG H+L coupled with alkaline phosphatase) (Sigma, St Louis, MO). The blot was developed with BCIP/NBT substrate. The positive control is rTDX1557 having a molecular weight of about 62 kDA. Lane 2 was left blank. The results are shown in FIGS. 3A and 3B.

As shown in FIG. 3A, SDA-PAGE analysis of feline lungworm A. abstrusus and heartworm lysates shows expression of 2M2 (TDX1557) in Feline lungworm. FIG. 3B, shows the immunoblot analysis of A. abstrusus lysate and heartworm lysate after probing with rabbit polyclonal antibodies against TDX1557 (Anti-TDX1557). Rabbit polyclonal antibodies against TDX1557 recognized a single band of ˜62 kDa of the A. abstrusus lysate but did not cross-react with the heartworm lysate.

### Example 4: No Cross-Reactivity of Anti-TDX1557 with Roundworm

This example illustrates that rabbit polyclonal antibodies raised against rTDX1557 did not cross-react with roundworm (Toxocara Spp) using SDS-PAGE and immunoblot analysis.

SDS-PAGE and Immunoblot analysis of A. abstrusus and roundworm lysates.

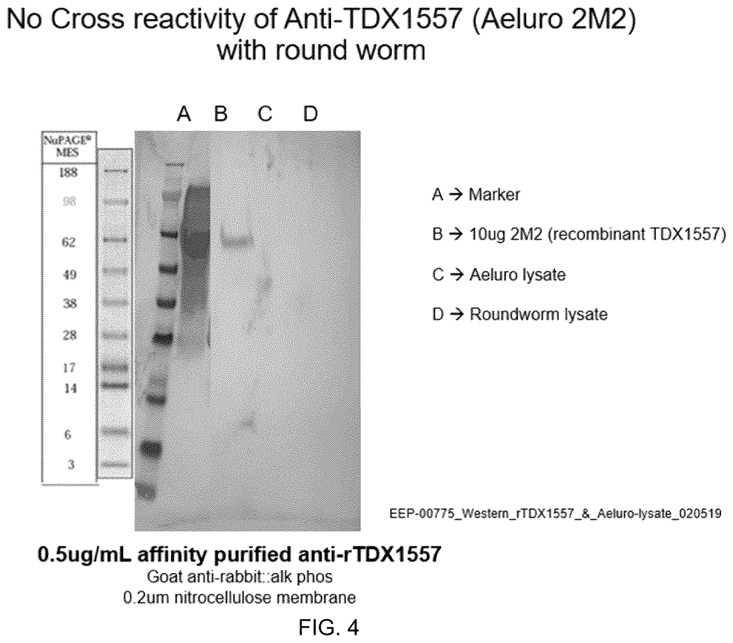

Rabbit polyclonal antibodies against rTDX1557, roundworm antigens (Toxocara spp) and lungworm antigens and were prepared as described above. In the SDS-PAGE gel, rTDX1557 (lane B), A. abstrusus lungworm lysate (lane C), and roundworm lysate (lane D), were individually mixed with SDS-PAGE buffer and ran on SDS-PAGE gel electrophoresis using conventional procedures. The resulting gel was subjected to immunoblot analysis by transferring the total proteins of the in the gels to NC membrane. The NC membrane was probed with rabbit polyclonal antibodies against TDX1557, followed by the addition of alkaline phosphatase conjugates (Anti rabbit IgG H+L coupled with alkaline phosphatase, Sigma, St. Louis, MO). The blot was developed with BCIP/NBT substrate (Sigma. St. Louis. MO). The positive control is rTDX1557. The results are shown in FIG. 4.

As shown in FIG. 4, shows an immunoblot analysis of A. abstrusus lysate and roundworm lysate probed with Rabbit polyclonal antibodies against TDX1557 (Anti-TDX1557) prepared as described above. Rabbit polyclonal antibodies against TDX1557 recognizes a single band of ˜62 kDa of the A. abstrusus lysate pellet (lane C) but did not cross-react with round worm soluble antigen (lane D).

### Example 5: Detection of Lungworm Infection Using ELISA and a SNAP® Test Using TDX1557

This example compares detection and measurement of lungworm infection using a fecal Baermann technique (results taken from Example 2) and indirect ELISA assay (results taken from Example 2) with the SNAP diagnostic device, using lungworm lysate and rTDX1557 as positive controls.

Experimental feline infection: A feline (BigMac) was orally infected with A. abstrusus larvae as described in Example 2. The fecal samples and blood draw were collected over a defined time period (Day 0, 7, 14, 21, 28, 35, 42, 49, 56, 63, 70, 77, 84, 91, 98, 112, 126, 140, 154, 168 and 182). The infected feline were treated with anti-parasitic drugs Imidacloprid and Moxidectin by Day 90. Baermann method for visual detection and for counting lungworm larvae was performed on the fecal samples using standard procedures (A J Carruth et al., Journal of Feline Medicine and Surgery, 2019, Vol. 5, pp. 1-6) The serum samples collected during the defined time period were evaluated in an indirect ELISA assay with lungworm lysate and rTDX1557 as controls and in a SNAP assay device using rTDX1557 as a control. A. abstrusus lysate was prepared from lungworm larvae as described above. The total protein in the lysate was determined by conventional Bradford assay. The recombinant protein TDX1557 was obtained as describe above.

ELISA Assays for A. abstrusus Lysate and rTDX1557.

Indirect ELISA assays for lungworm lysate and rTDX1557 were performed using the procedure described above. Two felines (BigMac and Burrito) were experimentally infected with lungworm and serum samples from these felines were diluted with PRRS buffer and were used for the ELISA procedure.

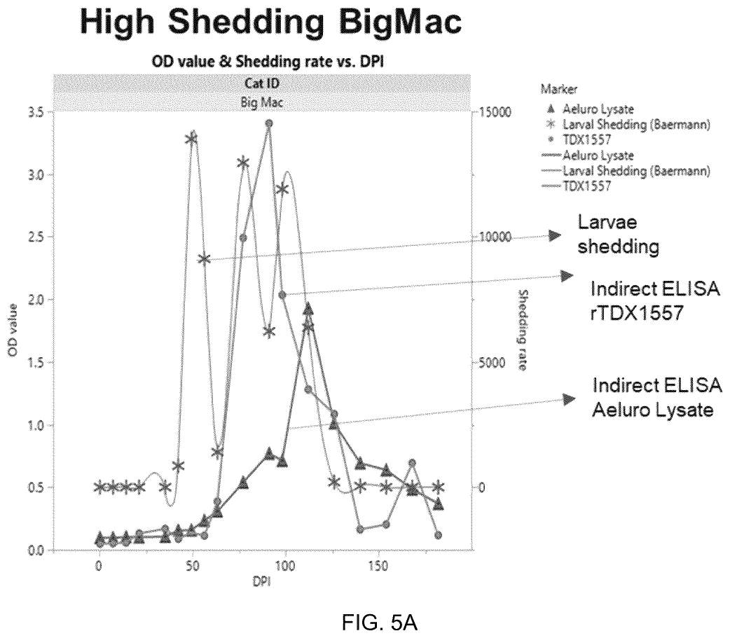

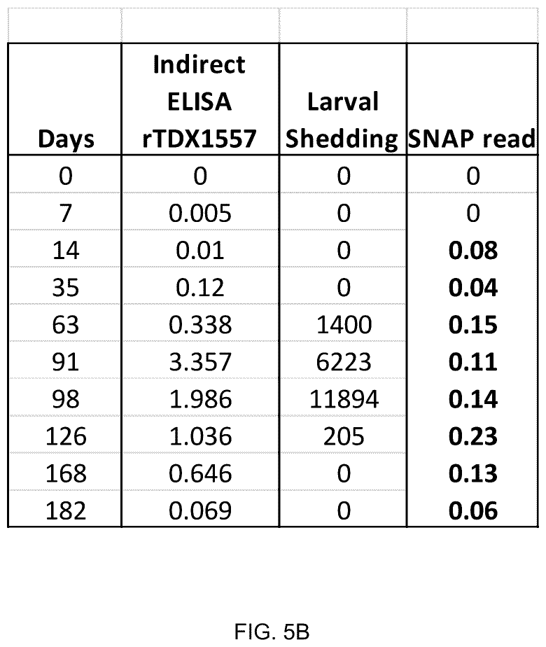

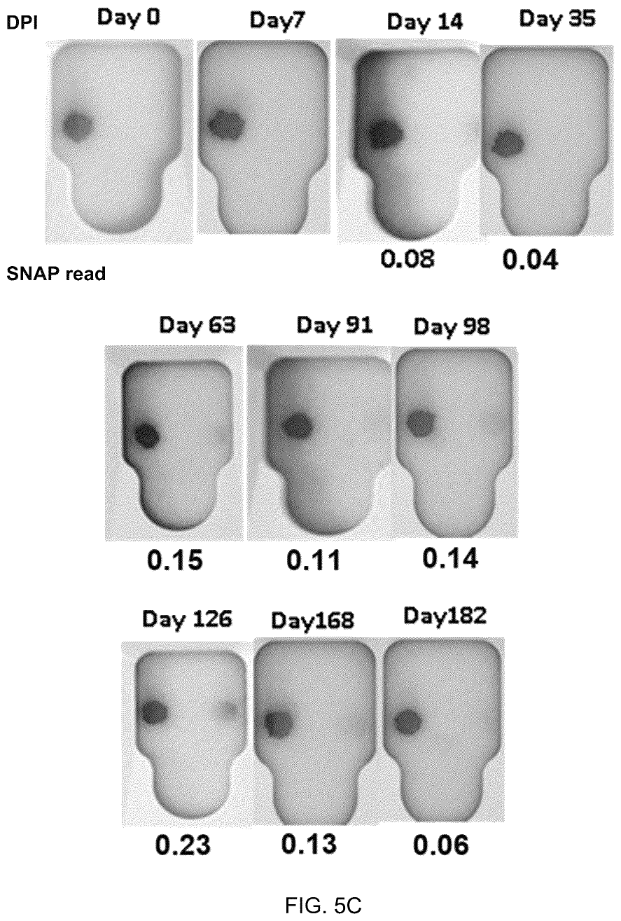

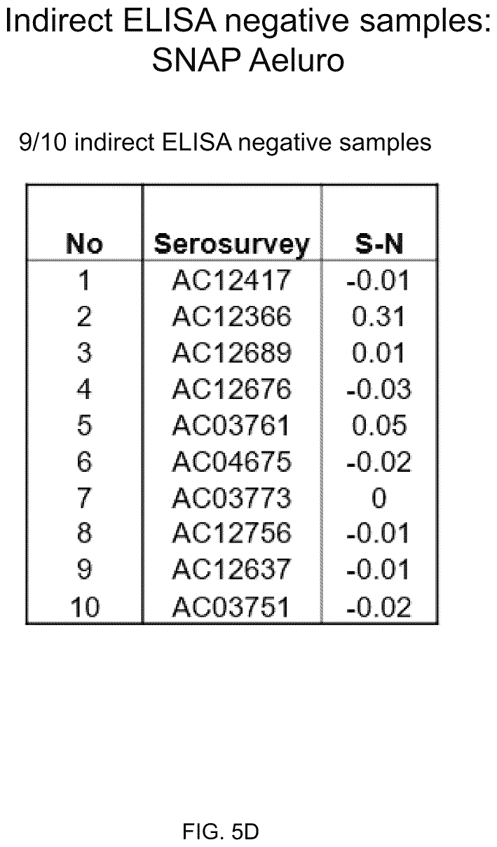

The lungworm lysate or rTDX1557 were coated on an Immulon IV plate for an overnight period at 4 C, and the plates were blocked with blocking solution (1% BSA in 100 mM Tris pH 7.5). The plates were washed with 5×PBST, and the serum samples from the experimentally infected feline (BigMAC) were diluted with PRRS diluent and added to the plates, and kept it for an hour at RT. The plates were washed again with 5×PBST, and the secondary antibody (Anti-Cat IgG H+L) was added to the plates and kept it for 30 minutes. The plates were washed, and the substrate was added and read at 450 nm. The results of the indirect ELISA assay using lungworm lysate and rTDX1557 as controls are shown in FIG. 5 A (reproduced from FIG. 2A).

### SNAP Assay

Recombinant TDX1557 was passively coated (1% solids) in PBS at 1 mg/mL on the polystyrene particles [Sphero, Lake Forest, IL] and 0.7 ul was spotted on the matrix [IDEXX Laboratories]. The positive control was goat anti HRPO coated latex particle. The SNAP device was built with wash buffer (IDEXX Laboratories]. The recombinant TDX1557 was also covalently linked with HRPO by periodate conjugation. The conjugate (180 ul) was mixed with 150 ul of serum sample and added to the sample well. Once the sample reaches the activation circle, the SNAP device was activated to wash the matrix, followed by development with the substrate. The SNAP result was read visually by naked eye and by densitometer. The SNAP results are shown in FIG. 5C. The larval shedding results and indirect ELISA assay are summarized in FIG. 5A (reproduced from FIG. 2A). As shown in FIG. 5A, a comparison of the fecal results using standard Baermann test showed inconsistent larval shedding relative to the indirect ELISA results using serum samples where the antibody response against rTDX1557 and A. abstrusus lysate showed steady progress over the time period.

The SNAP results using serum samples are summarized in FIG. 5C. Antibody reactivity was noted from day 14 and onwards.

A numerical comparison of the results of the Baermann test (larval shedding), indirect ELISA (with rTDX1557), and SNAP results are shown in FIG. 5B. Relative to the larval shedding and indirect ELISA assay, the SNAP assay results should detection of lungworm infection as early as day 14 from experimental infection. In contrast, the indirect ELISA and larval test showed detection of lungworm infection at day 35 and day 63 from experimental infection. SNAP results of ten feline field samples show a good correlation with indirect ELISA negative except one.

### Example 6: Construction, Expression and Characterization of Truncated Versions of TDX1557

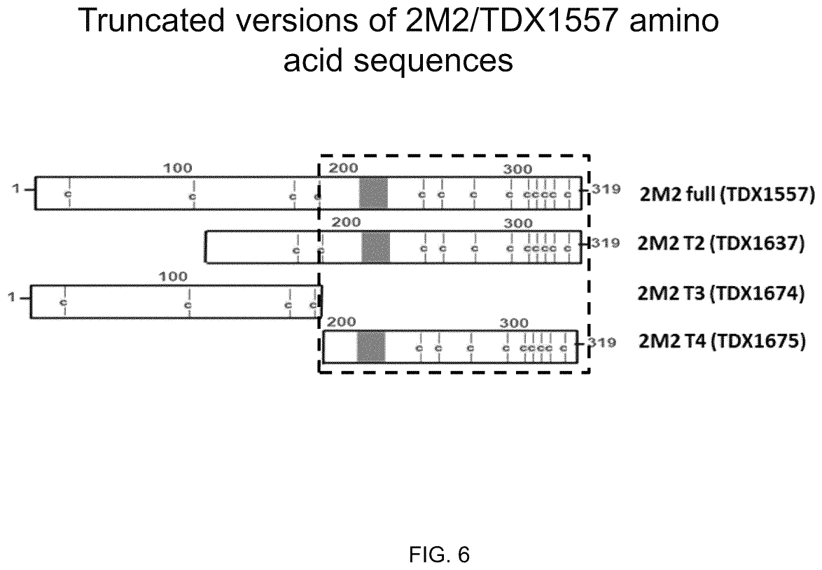

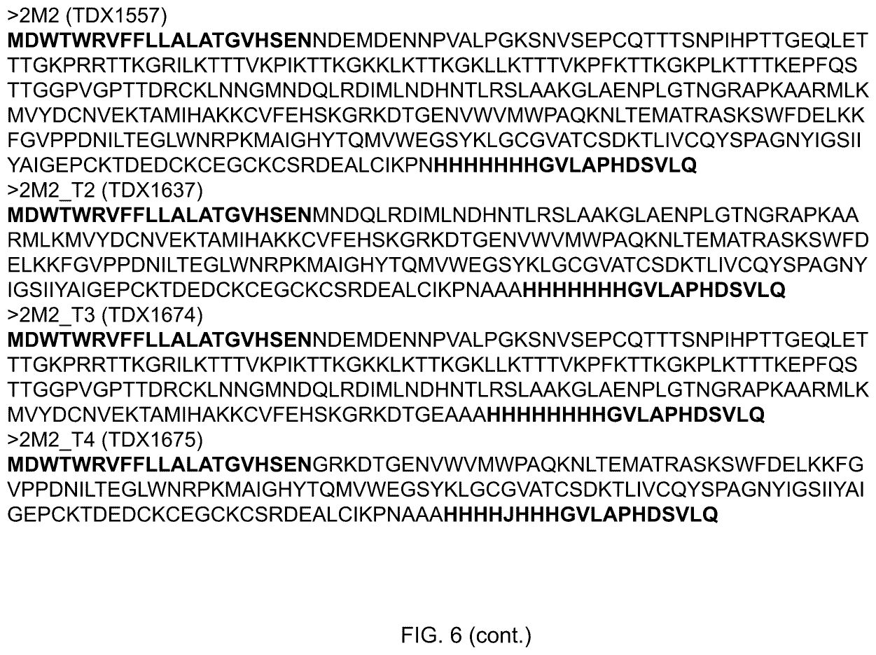

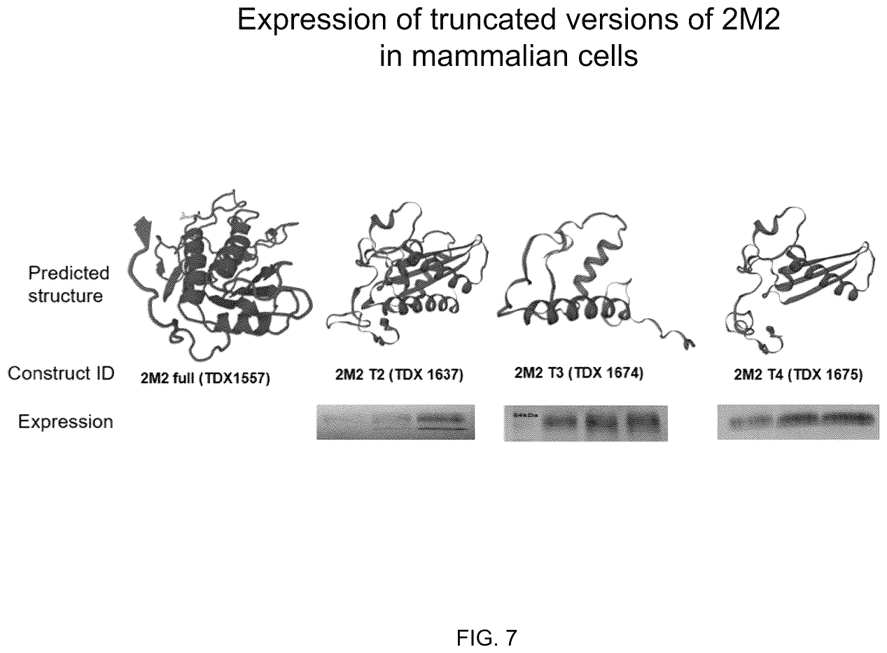

This example discusses the construction of truncated versions of 2M2/TDX1557 and evaluation of these truncated 2M2 peptides in an indirect ELISA assay using rabbit pAbs against 2M2, temporal (Burrito DPI 98, and French Fry DPI 91) and negative samples SPF 16CMF3, SPF 16CSL3). The amino acid sequences of four truncates are shown in FIG. 6. The truncated versions of TDX1557 labeled as T2 (also referred to as 2M2_T2 or TDX1637), T3 (also referred to 2M2_T3 or TDX1674) and T4 (also referred to as 2M2_T4 or TDX1675) were cloned and expressed as discussed above for rTDX1557.

CONSTRUCT FOR T2
(SEQ ID NO: 23)
ATGGACTGGACTTGGAGAGTGTTCTTCCTGCTGGCCCTGGCTACAGGCGTGCACAGCGAGAACATGAA
CGACCAGCTGCGGGACATCATGCTGAACGATCACAACACCCTGAGAAGCCTGGCCGCCAAAGGACTGG
CCGAAAATCCTCTGGGCACCAACGGCAGAGCCCCTAAAGCTGCCAGAATGCTGAAGATGGTGTACGAC
TGCAACGTGGAAAAGACCGCCATGATCCACGCCAAGAAATGCGTGTTCGAGCACAGCAAGGGCAGAAA
GGACACCGGCGAGAACGTGTGGGTTATGTGGCCCGCTCAGAAAAACCTGACCGAGATGGCCACCAGAG
CCAGCAAGAGTTGGTTCGACGAGCTGAAGAAATTCGGCGTGCCACCTGACAACATCCTGACCGAAGGC
CTGTGGAACAGACCCAAGATGGCCATTGGCCACTACACCCAGATGGTCTGGGAGGGCTCTTACAAGCT
CGGATGTGGCGTGGCCACATGCAGCGACAAGACCCTGATCGTGTGCCAGTATAGCCCTGCCGGCAACT
ACATCGGCAGCATCATCTATGCCATCGGCGAGCCCTGCAAGACCGACGAGGATTGCAAGTGCGAGGGC
TGCAAGTGCAGCAGAGATGAGGCCCTGTGCATCAAGCCTAATGCCGCCGCTCACCACCATCATCACCA
TCACCACGGTGTTCTGGCCCCTCACGATTCTGTTCTGCAGTAATAA
CONSTRUCT FOR T3
(SEQ ID NO: 24)
ATGGACTGGACTTGGAGAGTGTTCTTCCTGCTGGCCCTGGCTACAGGCGTGCACAGCGAGAACAACGA
CGAGATGGACGAGAACAATCCCGTGGCTCTGCCCGGCAAGAGCAATGTGTCTGAGCCTTGCCAGACCA
CCACCAGCAATCCTATTCACCCTACCACCGGCGAGCAGCTGGAAACCACAACAGGCAAGCCCAGACGG
ACCACCAAGGGCAGAATCCTGAAAACCACCACCGTGAAGCCCATCAAGACAACAAAGGGCAAGAAGCT
CAAGACGACCAAGGGGAAGCTGCTGAAAACAACAACAGTCAAACCCTTCAAGACCACGAAGGGAAAGC
CCCTTAAGACAACCACCAAAGAGCCCTTCCAGAGCACCACAGGTGGACCAGTGGGCCCAACCACCGAT
AGATGCAAGCTGAACAACGGCATGAACGACCAGCTGCGGGACATCATGCTGAACGATCACAACACCCT
GAGAAGCCTGGCCGCCAAAGGACTGGCCGAAAATCCTCTGGGCACCAACGGCAGAGCCCCTAAAGCTG
CCAGAATGCTGAAGATGGTGTACGACTGCAACGTGGAAAAGACCGCCATGATCCACGCCAAGAAATGC
GTGTTCGAGCACAGCAAAGGCCGGAAGGATACAGGCGAAGCCGCCGCTCACCACCATCATCACCATCA
CCACGGTGTTCTGGCCCCTCACGATTCTGTTCTGCAGTAATAA
CONSTRUCT FOR T4
(SEQ ID NO: 25)
ATGGACTGGACTTGGAGAGTGTTCTTCCTGCTGGCCCTGGCTACAGGCGTGCACTCTGAGAACGGCAG
AAAGGACACCGGCGAGAACGTGTGGGTTATGTGGCCCGCTCAGAAAAACCTGACCGAGATGGCCACCA
GAGCCAGCAAGAGTTGGTTCGACGAGCTGAAGAAATTCGGCGTGCCACCTGACAACATCCTGACCGAA
GGCCTGTGGAACAGACCCAAGATGGCCATTGGCCACTACACCCAGATGGTCTGGGAGGGCTCTTACAA
GCTCGGATGTGGCGTGGCCACATGCAGCGACAAGACCCTGATCGTGTGCCAGTATAGCCCTGCCGGCA
ACTACATCGGCAGCATCATCTATGCCATCGGCGAGCCCTGCAAGACCGACGAGGATTGCAAGTGCGAG
GGCTGCAAGTGCAGCAGAGATGAGGCCCTGTGCATCAAGCCTAATGCCGCCGCTCACCACCATCATCA
CCATCACCACGGTGTTCTGGCCCCTCACGATTCTGTTCTGCAGTAATAA

While making the construct, an IgG-like signal sequence

(SEQ ID NO: 21)
MDWTWRVFFLLALATGVHSEN

was included at positions 1 to 63 of SEQ ID NO:23 (T2 CONSTRUCT), positions 1 to 63 of SEQ ID NO:24 (T3 CONSTRUCT), and positions 1 to 63 of SEQ ID NO:25 (T4 CONSTRUCT) at the amino-terminal regions of each truncate to secrete the truncates into the supernatant. The C-terminal region was tagged with HIS tags (AAAHHHHHHHGVLAPHDSVLQ) (SEQ ID NO:22) to facilitate the purification of recombinant truncated proteins. The recombinant constructs was transfected into a mammalian expression system (HEK293 cells) using ExpiFectamine™ transfection kit (Thermo Fisher Scientific, Carlsbad, CA). The supernatant was collected after 5-7 days and the recombinant truncated proteins were purified using immobilized metal affinity chromatography (IMAC). The predicted structure [https://wlab.ethz.ch/protter/] indicates the secondary structure such as helical and beta sheets of the truncated versions. SDS-PAGE analysis shows the expression of each truncated TDX1557 protein.

2M2 (TDX1557).
(SEQ ID NO: 3)
MDWTWRVFFLLALATGVHSENNDEMDENNPVALPGKSNVSEPCQTTTSNPIHPTTGEQLETTTG
KPRRTTKGRILKTTTVKPIKTTKGKKLKTTKGKLLKTTTVKPFKTTKGKPLKTTTKEPFQSTTGGP
VGPTTDRCKLNNGMNDQLRDIMLNDHNTLRSLAAKGLAENPLGTNGRAPKAARMLKMVYDC
NVEKTAMIHAKKCVFEHSKGRKDTGENVWVMWPAQKNLTEMATRASKSWFDELKKFGVPPD
NILTEGLWNRPKMAIGHYTQMVWEGSYKLGCGVATCSDKTLIVCQYSPAGNYIGSITYAIGEPCK
TDEDCKCEGCKCSRDEALCIKPNHHHHHHHGVLAPHDSVLQ
2M2_T2 (TDX1637)
(SEQ ID NO: 4)
MDWTWRVFFLLALATGVHSENMNDQLRDIMLNDHNTLRSLAAKGLAENPLGTNGR
APKAARMLKMVYDCNVEKTAMIHAKKCVFEHSKGRKDTGENVWVMWPAQKNLTEMATRAS
KSWFDELKKFGVPPDNILTEGLWNRPKMAIGHYTQMVWEGSYKLGCGVATCSDKTLIVCQYSP
AGNYIGSIIYAIGEPCKTDEDCKCEGCKCSRDEALCIKPNAAAHHHHHHHGVLAPHDSVLQ
>2M2_T3 (TDX1674)
(SEQ ID NO: 5)
MDWTWRVFFLLALATGVHSENNDEMDENNPVALPGKSNVSEPCOTTTSNPIHPTTG
EQLETTTGKPRRTTKGRILKTTTVKPIKTTKGKKLKTTKGKLLKTTTVKPFKTTKGKPLKTTTKEP
FQSTTGGPVGPTTDRCKLNNGMNDQLRDIMLNDHNTLRSLAAKGLAENPLGINGRAPKAARML
KMVYDCNVEKTAMIHAKKCVFEHSKGRKDTGEAAAHHHHHHHHGVLAPHDSVLQ
>2M2_T4 (TDX1675)
(SEQ ID NO: 6)
MDWTWRVFELLALATGVHSENGRKDTGENVWVMWPAQKNLTEMATRASKSWED
ELKKFGVPPDNILTEGLWNRPKMAIGHTQMVWEGSYKLGCGVATCSDKTLIVCQYSPAGNYIGS
IIYAIGEPCKTDEDCKCEGCKCSRDEALCIKPNAAAHHHHHHHHGVLAPHDSVLQ

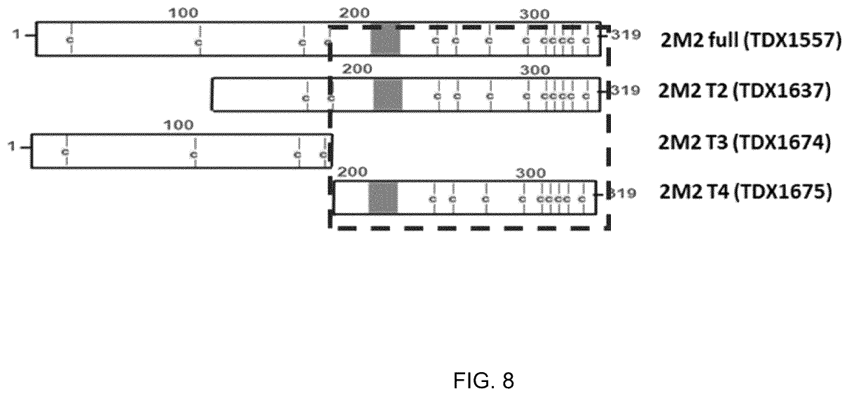

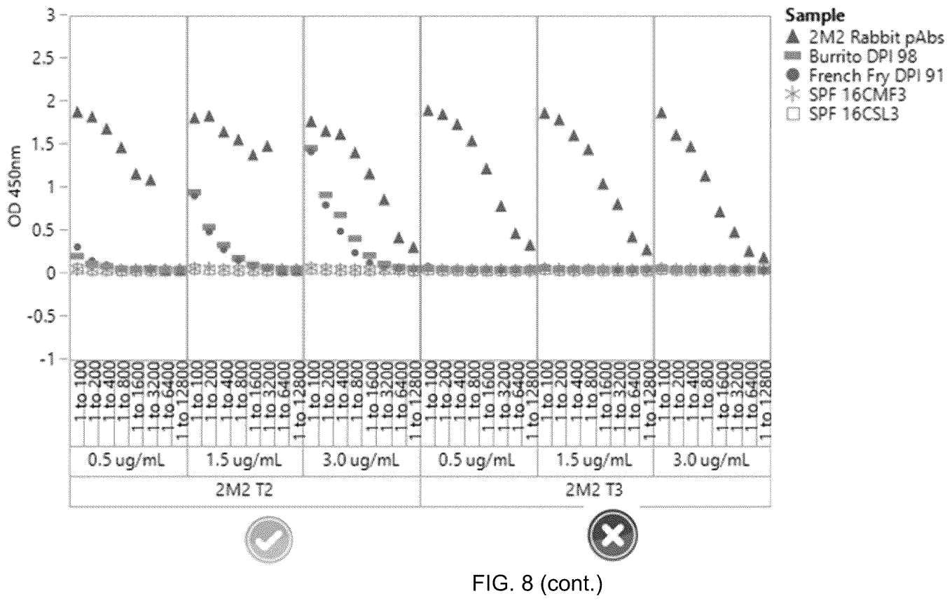

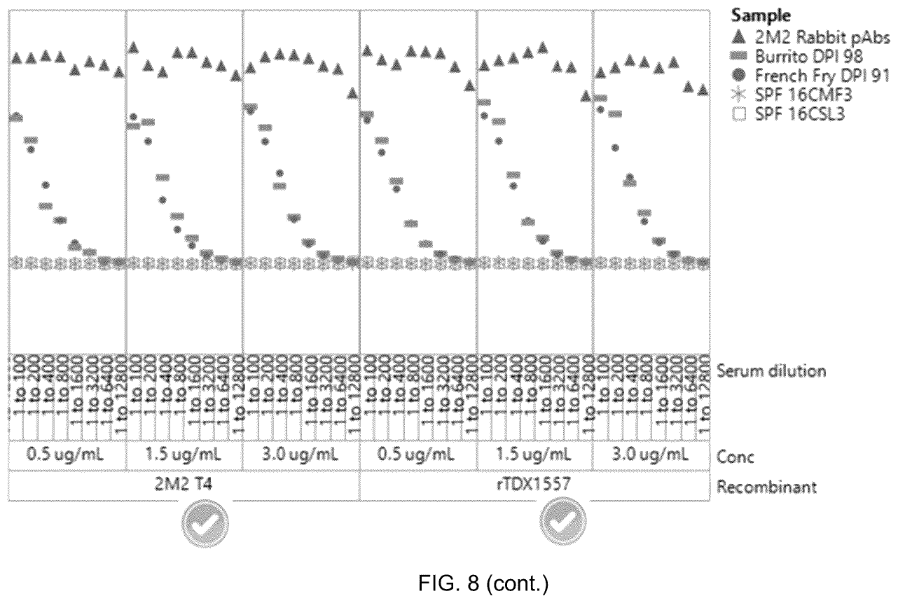

The indirect ELISA procedure utilized in this Example was described above. Briefly, rTDX1557 and the recombinant truncates T2, T3 and T4 were each coated on separate Immulon IV plates for an overnight period at 4 C, and the plates were blocked with blocking solution (1% BSA in 100 mM Tris pH 7.5). The plates were washed with 5×PBST, and the serum samples from experimentally infected cats were diluted with PRRS diluent and added to the plates, and the plates were kept for an hour at RT. The plates were washed again with 5×PBST, and the secondary antibody (Anti-Cat IgG H+L) was added to the plates and kept it for 30 minutes. The plates were washed, and the substrate was added and read at 450 nm. The results are shown in FIG. 8.

FIG. 8 shows an evaluation of truncated versions of 2M2 (TDX1557) using indirect ELISA assay. Four (4) different versions of recombinant peptides (2M2 full, T2, T3 and T4) were evaluated in an indirect ELISA using rabbit pAbs, temporal (Burrito DPI 98, French Fry DPI 91) and negative samples (SPF 16CMF3, SPF 16CSL3). The serially diluted serum samples were tested at different concentrations (0.5, 1.5 and 3 ug/ml) of the recombinant peptides. The rabbit polyclonal antibodies against TDX1557 reacted with all the four different recombinant peptides. However, the experimentally infected samples did not show any reactivity with 2M2 T3 truncate, indicating that the significant epitope is present only in the protein's carboxy-terminal. Specific pathogen free (SPF) negative controls (SPF Cat ID 16CMF3 and 16CSL3) show no reactivity to any of the recombinant peptides. Based on the antigen concentration and the dilution rate, an increase in titration rate was also observed indicating the antibody response against the truncated versions.

Truncates 2M2 Amino2 Cys, 2M2 CpepC, 2M2 Wo epitope and truncated Cys-Ser variants of 2M2 T4 (referred to as 2M2 T4 WO IDR, 2M2 T4 1A, 2M2 T4 1B, and 2M2 T4 1C) were also constructed. See FIG. 16C. The sequences for these truncates are shown in FIG. 16D. Truncates 2M2 Amino2 Cys, 2M2 CpepC were successfully expressed but not the truncated Cys-Ser variants. FIGS. 16E and 16F shows an evaluation of 2M2 Amino2 Cys, 2M2 CpepC, 2M2 Wo epitope using the procedures described above. As shown in FIGS. 16E and 16F, no reactivity to temporal samples were found.

### Example 7 A: Identification of Epitopes of TDX1557 Using Peptide Array

This example shows an evaluation of a peptide array with serum samples from non-infected felines (TDX 1557 ELISA negatives) based on an ELISA protocol on a Neutravidin plate described above. The array included 62 peptides of 15 mers with 10 amino acids overlap, covering the entire full length 2M2 protein.

| SEQ ID | | |
|---|---|---|
| NO: | Peptide ID | Peptide Sequence |
| 27 | A01 | NDEMDENNPVALPGK |
| 28 | A02 | ENNPVALPGKSNVSE |
| 29 | A03 | ALPGKSNVSEPCQTT |
| 30 | A04 | SNVSEPCQTTTSNPI |
| 31 | A05 | PCQTTTSNPIHPTTG |
| 32 | A06 | TSNPIHPTTGEQLET |
| 33 | A07 | HPTTGEQLETTTGKP |
| 34 | A08 | EQLETTTGKPRRTTK |
| 35 | A09 | TTGKPRRTTKGRILK |
| 36 | A10 | RRTTKGRILKTTTVK |
| 37 | A11 | GRILKTTTVKPIKTT |
| 38 | A12 | TTTVKPIKTTKGKKL |
| 39 | B01 | PIKTTKGKKLKTTKG |
| 40 | B02 | KGKKLKTTKGKLLKT |
| 41 | B03 | KTTKGKLLKTTTVKP |
| 42 | B04 | KLLKTTTVKPFKTTK |
| 43 | B05 | TTVKPFKTTKGKPLK |
| 44 | B06 | FKTTKGKPLKTTTKE |
| 45 | B07 | GKPLKTTTKEPFOST |
| 46 | B08 | TTTKEPFQSTTGGPV |
| 47 | B09 | PFQSTTGGPVGPTTD |
| 48 | B10 | TGGPVGPTTDRCKLN |
| 49 | B11 | GPTTDRCKLNNGMND |
| 50 | B12 | RCKLNNGMNDQLRDI |
| 51 | C01 | NGMNDQLRDIMLNDH |
| 52 | C02 | QLRDIMLNDHNTLRS |
| 53 | C03 | MLNDHNTLRSLAAKG |
| 54 | C04 | NTLRSLAAKGLAENP |
| 55 | C05 | LAAKGLAENPLGING |
| 56 | C06 | LAENPLGINGRAPKA |
| 57 | C07 | LGTNGRAPKAARMLK |
| 58 | C08 | RAPKAARMLKMVYDC |
| 59 | C09 | ARMLKMVYDCNVEKT |
| 7 | C10 | MVYDCNVEKTAMIHA |
| 8 | C11 | NVEKTAMIHAKKCVF |
| 9 | C12 | AMIHAKKCVFEHSKG |
| 10 | D01 | KKCVFEHSKGRKDTG |
| 11 | D02 | EHSKGRKDTGENVWV |
| 12 | D03 | RKDTGENVWVMWPAQ |
| 13 | D04 | ENVWVMWPAQKNLTE |
| 14 | D05 | MWPAQKNLTEMATRA |
| 15 | D06 | KNLTEMATRASKSWF |
| 16 | D07 | MATRASKSWFDELKK |
| 17 | D08 | SKSWFDELKKFGVPP |
| 60 | D09 | DELKKFGVPPDNILT |
| 61 | D10 | FGVPPDNILTEGLWN |
| 62 | D11 | DNILTEGLWNRPKMA |
| 63 | D12 | EGLWNRPKMAIGHYT |
| 64 | E01 | RPKMAIGHYTQMVWE |
| 65 | E02 | IGHYTQMVWEGSYKL |
| 66 | E03 | QMVWEGSYKLGCGVA |
| 67 | E04 | GSYKLGCGVATCSDK |
| 68 | E05 | GCGVATCSDKTLIVC |
| 69 | E06 | TCSDKTLIVCQYSPA |
| 70 | E07 | TLIVCQYSPAGNYIG |
| 71 | E08 | QYSPAGNYIGSIIYA |
| 72 | E09 | GNYIGSIIYAIGEPC |
| 73 | E10 | SIIYAIGEPCKTDED |
| 74 | E11 | IGEPCKTDEDCKCEG |
| 75 | E12 | KTDEDCKCEGCKCSR |
| 76 | F01 | CKCEGCKCSRDEALC |
| 77 | F02 | CKCSRDEALCIKPN |

These peptides were synthesized and labeled with biotin at the N-terminus using standard procedures (New England peptide, MA). The biotinylated peptides were coated in neutravidin plate (Thermofisher, Carlsbad, CA). Plate was incubated with serum samples followed by washing with PBST. The secondary antibody with HRP conjugate (Anti Cat Ig G H+L) was added and kept it for 30 minutes at room temperature. The plate was washed with PBST and the substrate was added. The stop solution was added, and the solution was read at 450 nm.

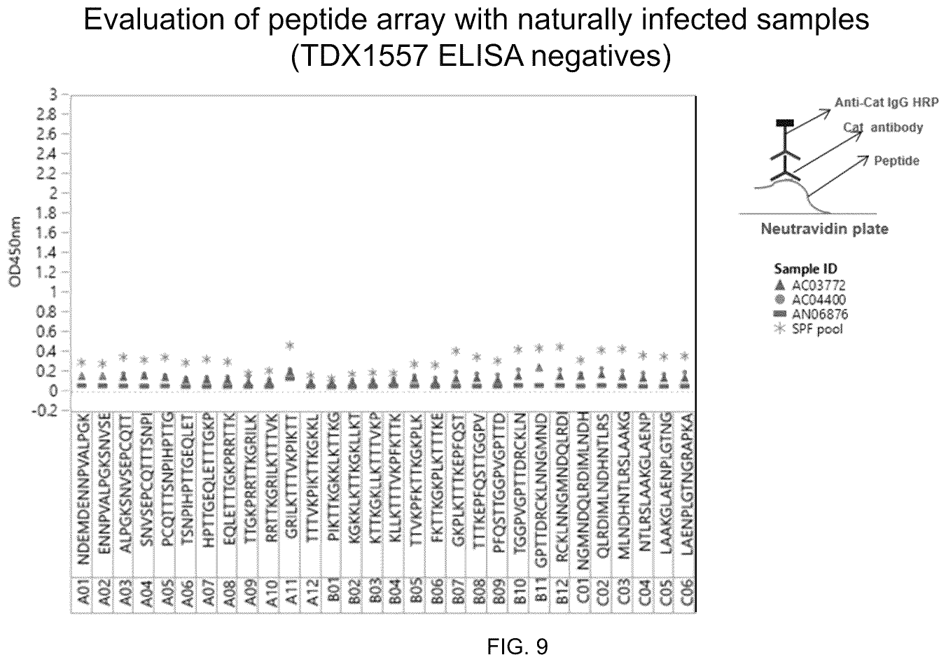

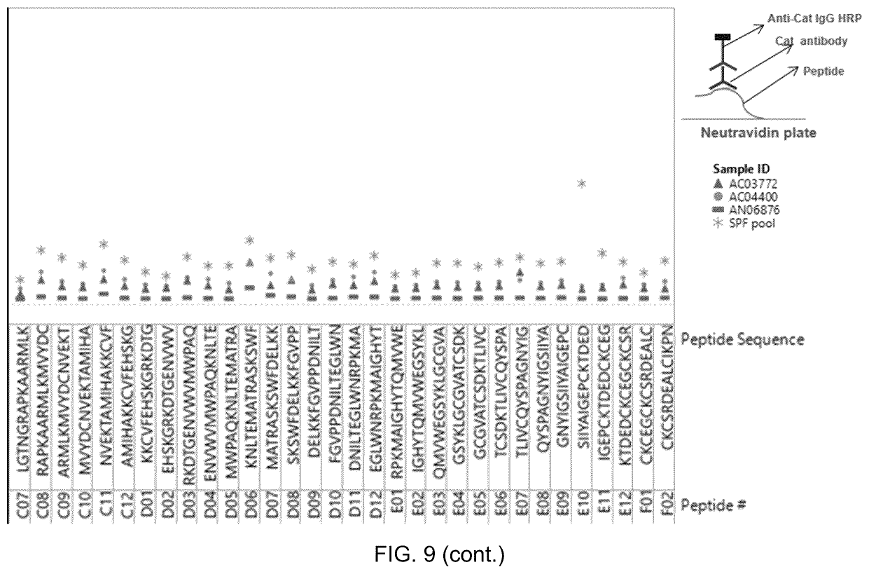

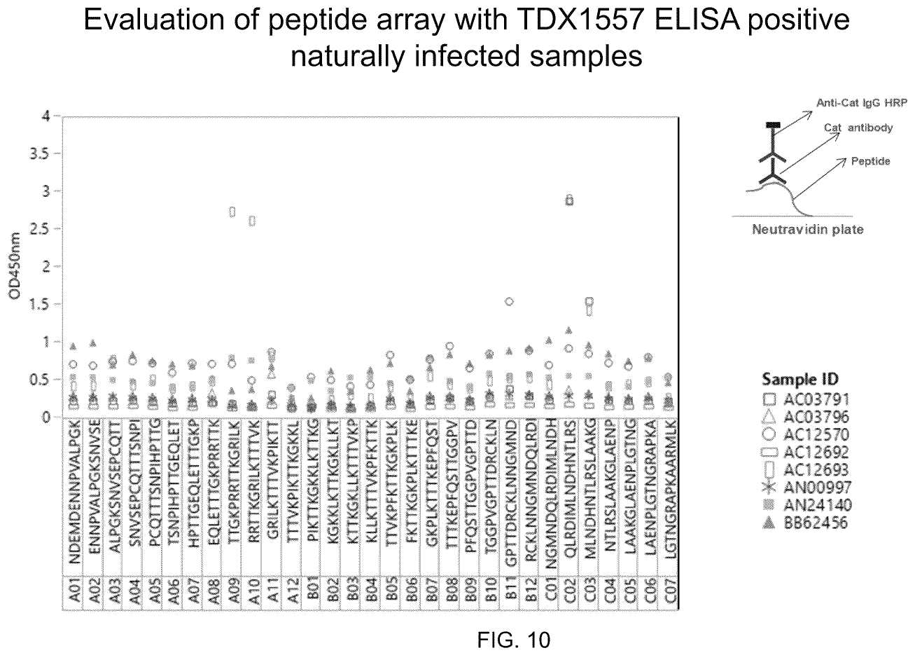

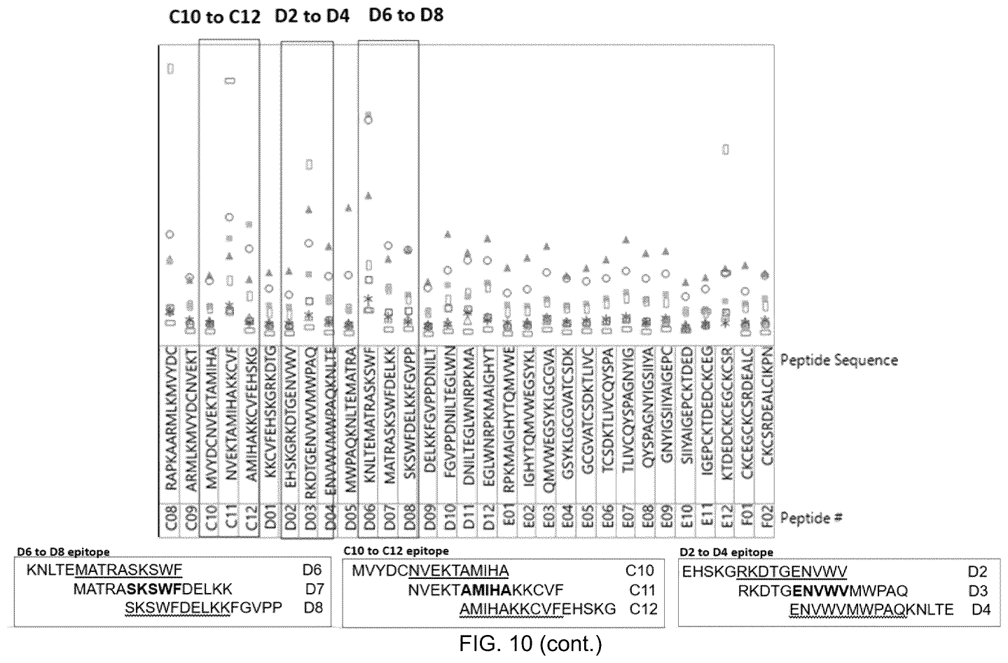

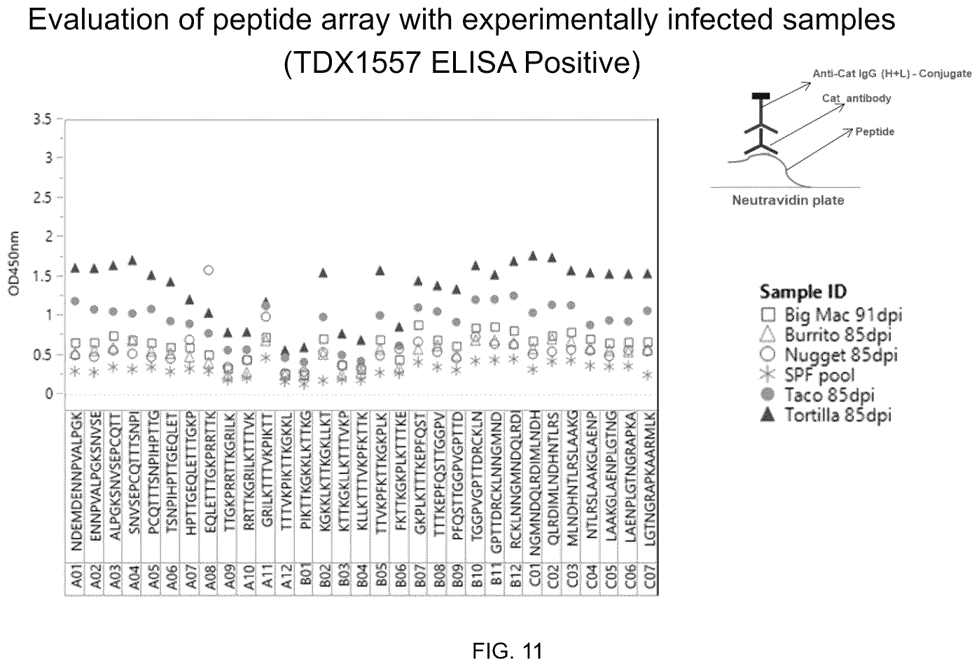

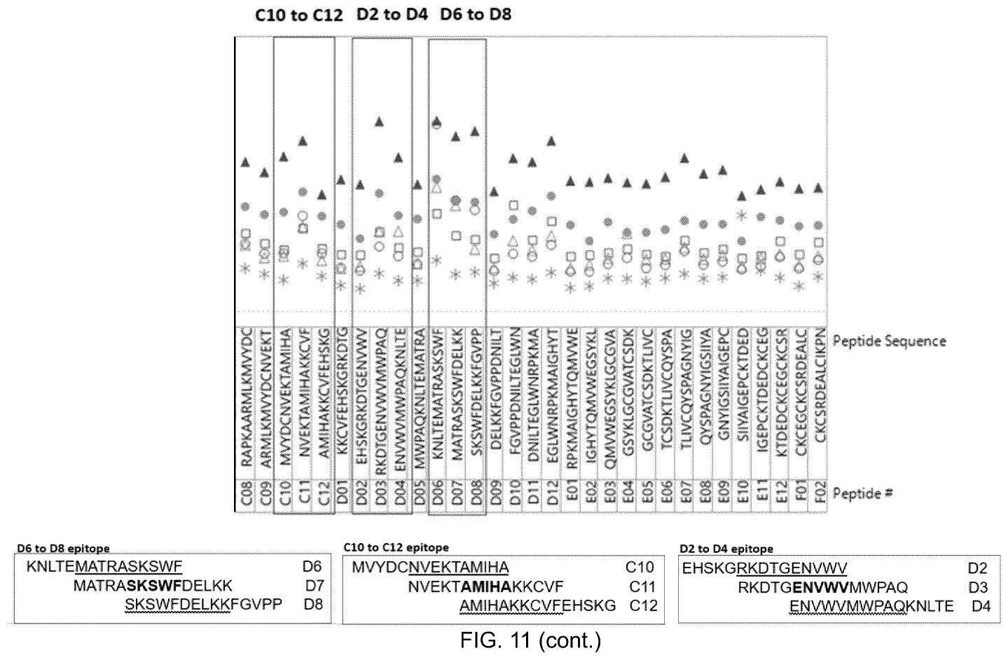

As shown in FIG. 9, the negative samples (AC03772, AC04400 and AN06876) and SPF Pool sample were utilized. The raw data was tabulated between negative and positive samples directly to show the differences. As far as the experimental or temporal samples, the OD value for day 0 (pre-bleed) was used to normalize the data for that particular cat. The negative samples show no significant reactivity to the peptides compared to positive field samples (AC03791. AC03796, AC12570, AC12692, AC12693, AN00997, AN24140 and BB62456) in FIG. 10 or experimentally infected samples (BigMac 91 dpi, Burrito 85 dpi, Nugget 85 dpi, Taco dpi85 and Tortilla dpi85) in FIG. 11.

### Example 7 B: Identification of Epitopes of TDX1557 Using Peptide Array

This example shows an evaluation of a peptide array of Example 7A with serum samples from naturally infected felines (TDX 1557 ELISA positive) using an ELISA protocol on a neutravidin plate as described above. The protein array was evaluated using serum samples taken from naturally infected felines (which show positive in an ELISA assay with complete protein). The results show significant reactivity to the peptides.

The N-terminal biotinylated peptides of Example 7A were coated in neutravidin plate using the procedure described above. Plate was incubated with serum samples followed by washing with PBST. The secondary antibody with HRP conjugate (Anti Cat Ig G H+L) was added and kept it for 30 minutes at room temperature. The plate was washed with PBST and the substrate was added. The stop solution was added, and the solution was read at 450 nm. The results are shown in FIG. 10.

As shown in FIG. 10, the positive cat serum samples showed significant reactivity to the following peptide sequences. These peptide sequences were found to be the most immunogenic peptides of TDX1557:

| Peptide # | Peptide Sequence |
|---|---|
| C10 | MVYDCNVEKTAMIHA (SEQ ID NO: 7) |
| C11 | NVEKTAMIHAKKCVF (SEQ ID NO: 8) |
| C12 | AMIHAKKCVFEHSKG (SEQ ID NO: 9) |
| D01 | KKCVFEHSKGRKDTG (SEQ ID NO: 10) |
| D02 | EHSKGRKDTGENVWV (SEQ ID NO: 11) |
| D03 | RKDTGENVWVMWPAQ (SEQ ID NO: 12) |
| D04 | ENVWVMWPAQKNLTE (SEQ ID NO: 13) |
| D05 | MWPAQKNLTEMATRA (SEQ ID NO: 14) |
| D06 | KNLTEMATRASKSWF (SEQ ID NO: 15) |
| D07 | MATRASKSWFDELKK (SEQ ID NO: 16) |
| D08 | SKSWFDELKKFGVPP (SEQ ID NO: 17) |

### Example 7 C: Identification of Immunodominant Epitopes of TDX1557 Using Peptide Array

This example shows an evaluation of a peptide array of Example 7A with serum samples taken from experimentally infected felines (TDX 1557 ELISA positive) using an ELISA protocol on a neutravidin plate as described above.

The N-terminally biotinylated peptides of Example 7A were coated on a neutravidin plate (Thermofisher, Carlsbad, CA) using the procedure described above. Plate was incubated with serum samples followed by washing with PBST. The secondary antibody with HRP conjugate (Anti Cat IgG H+L) was added and kept it for 30 minutes at room temperature. The plate was washed with PBST and the substrate was added. The stop solution was added, and the solution was read at 450 nm. The results are shown in FIG. 11.

As shown in FIG. 11, the peptide array of 2M2 were evaluated with eight positive experimentally infected samples (Big Mac 91 dpi, Burrito 85 dpi, Nugget 85 dpi, Taco 85 dpi, Tortilla 85 dpi) and Specific Pathogen Free (SPF) feline pool (Negative control). The experimentally infected samples which show significant positive reactivity in an ELISA assay with complete protein also showed significant reactivity to the peptides. The immunogenic peptide sequences (D6, D7, D8, C10, C11, C12, D2, D3, and D4) of Example 7B were found to be the most immunogenic.

FIG. 11 also includes an alignment of D6, D7, and 8, wherein the underling delineates an overlapping region shared by the three peptides, having the sequence SKSWF (SEQ ID NO:81) as containing an epitope. The region of overlap between D6 and D7 indicates that the sequence MATRASKSWF (SEQ ID NO:82) contains an epitope. The region of overlap between D7 and D8 indicates that the sequence SKSWFDELKK (SEQ ID NO:83) contains an epitope.

FIG. 11 further includes an alignment of C10, C11 and C12, wherein the underlining delineates an overlapping region shared by the three peptides, having the sequence AMIHA (SEQ ID NO:84) as a containing an epitope. The region of overlap between C10 and C11 indicates that the sequence NVEKTAMIHA (SEQ ID NO:85) contains an epitope. The region of overlap between C11 and C12 indicates that the sequence AMIHAKKCVF (SEQ ID NO:86) contains an epitope.

FIG. 11 additionally includes an alignment of D2, D3 and D4, wherein the overlapping region shared by the three peptides, having the sequence ENVWV (SEQ ID NO:87), contains an epitope. The region of overlap between D2 and D3 indicates that the sequence RKDTGENVWV (SEQ ID NO:88) contains an epitope. The region of overlap between D3 and D4 indicates that the sequence ENVWVMWPAQ (SEQ ID NO:89) contains an epitope.

### Example 8: Evaluation of Polypeptides D6, D7 and D8

This example shows a comparison of the reactivity of D6, D7 and D8 peptides, A. abstrusus lysate, and rTDX1557 with antisera from an experimentally infected feline. The peptides (D6, D7, D8) were prepared as described in Example 7A.

Antibody binding of D16, D7 and D8 peptides, A. abstrusus lysate and rTDX1557 was measured using an indirect ELISA assay. The linear peptides (D6, D7, D8) bound antibodies at early time points, indicating their diagnostic utility for detection and diagnosis of infection, including early stages of infection. ELISA assay with larval lysates and rTDX1557 showed a gradual increase overtime, consistent with a gradual increase in antibody titer. The antibody titer fell after treatment with anti-parasitic drug, which occurred at DPI 90. The Table below shows the OD values.

| | Lungworm | TDX1557 | D6 | D7 | D8 |
|---|---|---|---|---|---|
| DPI | lysate | (OD | (OD | (OD | (OD |
| (Days) | (OD value) | value) | value) | value) | value) |
| 0 | 0 | 0 | 0 | 0 | 0 |
| 7 | 0 | 0.034 | 0.149 | 0.107 | 0.132 |
| 14 | 0 | 0.019 | 0.019 | 0.012 | 0.062 |
| 21 | 0.015 | 0.028 | 0.366 | 0.247 | 0.332 |
| 28 | 0 | 0.029 | 0.203 | 0.223 | 0.275 |
| 35 | 0 | 0.037 | 0.712 | 0.588 | 0.709 |
| 42 | 0.098 | 0.028 | 1.433 | 1.36 | 1.307 |
| 49 | 0.072 | 0.162 | 1.526 | 1.525 | 1.463 |
| 56 | 0.151 | 0.458 | 1.982 | 1.917 | 1.863 |
| 63 | 0.253 | 0.487 | 1.358 | 1.256 | 1.257 |
| 70 | 0.311 | 0.698 | 1.146 | 1.072 | 1.249 |
| 77 | 0.29 | 0.583 | 1.246 | 1.163 | 1.196 |
| 85 | 0.338 | 0.522 | 0.783 | 0.838 | 0.796 |
| 91 | 1.235 | 1.201 | 0.675 | 0.803 | 0.904 |
| 98 | 0.747 | 1.081 | 0.425 | 0.465 | 0.614 |
| 112 | 0.198 | 0.062 | 0.62 | 0.662 | 0.811 |
| 126 | 0.224 | 0.045 | 0.749 | 0.778 | 0.846 |
| 140 | 0.114 | 0.063 | 0.531 | 0.433 | 0.646 |
| 154 | 0 | 0.055 | 0.588 | 0.459 | 0.797 |
| 168 | 0 | 0.064 | 0.097 | 0.219 | 0.47 |
| 182 | 0 | 0 | 0.445 | 0.765 | 1.464 |

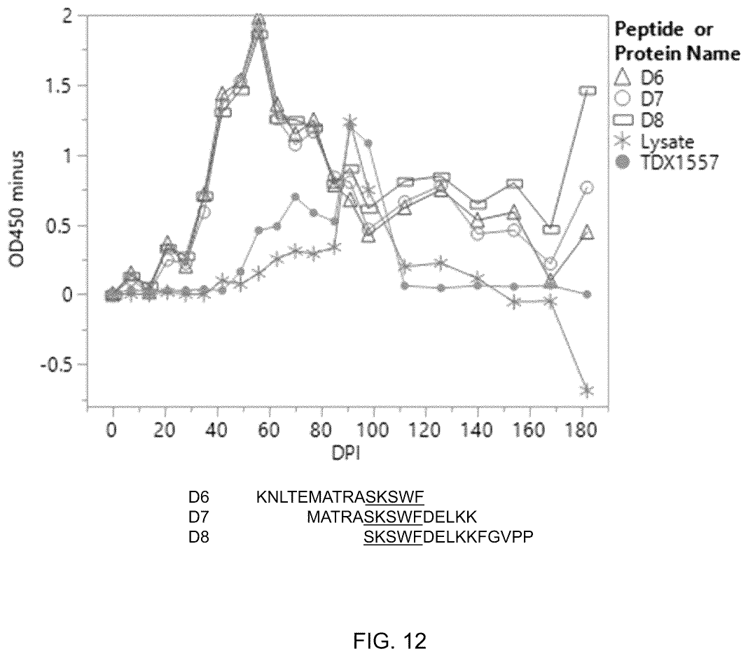

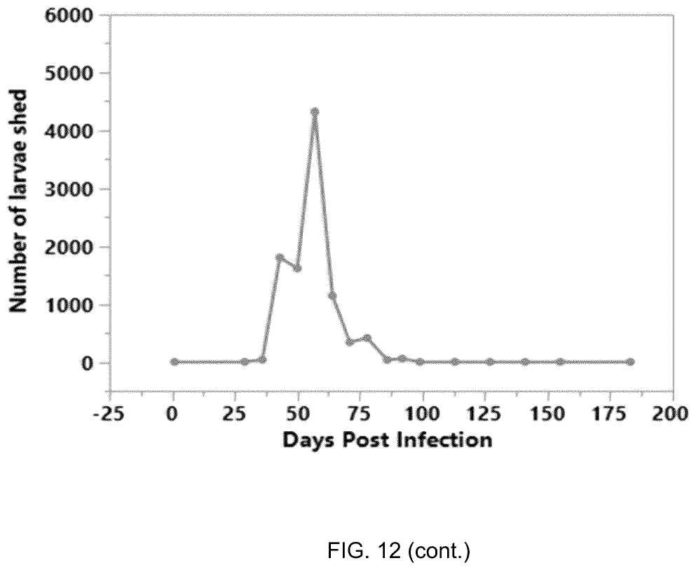

FIG. 12 shows the comparison of ELISA assay with peptides (D6, D7 & D8), recombinant TDX1557 and A. abstrusus lungworm lysate using serum samples from experimentally infected high shedding cat, Tortilla.

### Example 9: Evaluation of D678 Polypeptide in Infected Felines

This example describes the construction of D678 polypeptide and evaluation of its reactivity with serum samples taken from experimentally infected felines.

FIG. 13 shows a polypeptide structure created using Protter (https://wlab.ethz.ch/protter/), an open-source tool used for visualization of protein form, for the construction of 2M2. Protter indicated that 2M2 is a secretory protein and includes underlined regions of immunogenic peptides D6, D7 and D8 within the 2M2 sequence. An alignment of D06, D07 and DOS shows an overlapping SKSWF sequence (SEQ ID NO:81). A combination peptide D678 (“D678 combo”), based on the combination of D6, D7, and D8 peptides was synthesized using standard peptide synthesis procedures.

CPAQKNLTEMATRASKSWFDELKKFG D678 combo (SEQ ID NO:18)

The N-terminal C residue of SEQ ID NO:18 is not part of the natural sequence. It was added for ease of manipulation, e.g., conjugation, as is well known in the art. “D678 modified”, i.e., SEQ ID NO:78 below, is the same as SEQ ID NO: 18 except that it does not have the N-terminal C.

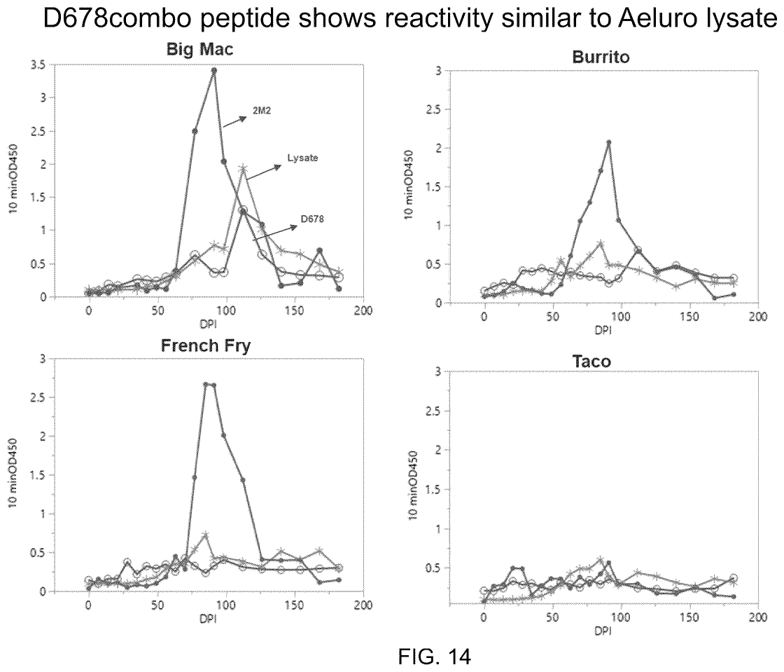

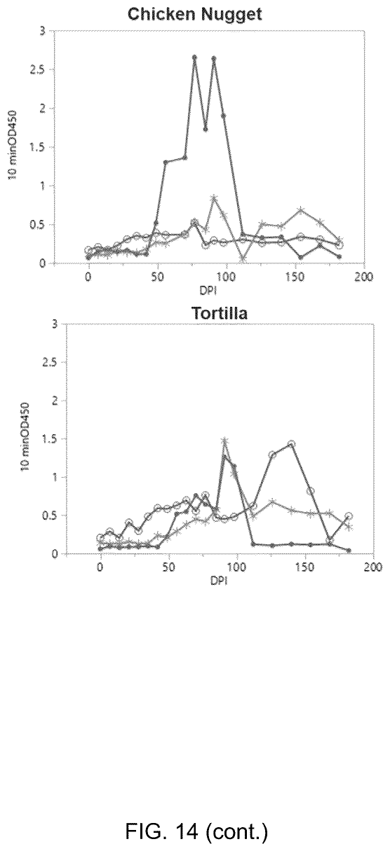

The A. abstrusus lungworm lysate prepared as described above, rTDX1557 prepared as described above, and non-biotinylated synthetic peptide D678 were separately coated onto Immulon IV plates for an overnight period at 4 C, and the plates were blocked with blocking solution (1% BSA in 100 mM Tris pH 7.5). The plates were washed with 5×PBST, and serum samples from the experimentally infected felines were diluted with PRRS diluent and added to the plates, and incubated for one hour at RT. The plates were washed again with 5×PBST, and a secondary antibody (Anti-Cat IgG H+L) was added to the plates and incubated for 30 minutes at RT. The plates were washed, substrate was added and the plates were read at 450 nm. The results of the indirect ELISA assay using lungworm lysate and rTDX1557 as controls and peptide D678 are shown in FIG. 14.

Although peptide D678 is the combined version of immunogenic D6, D7, and D8 peptides which produces an early antibody response as discussed and shown in Examples 8 and 9, peptide D678 was found not to be equivalent in terms of its antibody response relative to individual peptides (D6, D7, and D8). The lack of a high antibody response might be due to non-biotinylated form of D678 being evaluated relative to the biotinylated D6, D7 and D8 peptides used in the experiments discussed above.

### Example 10: Construction of Extended D678 Polypeptide and Evaluation of Detection of Antibodies from Infected Felines

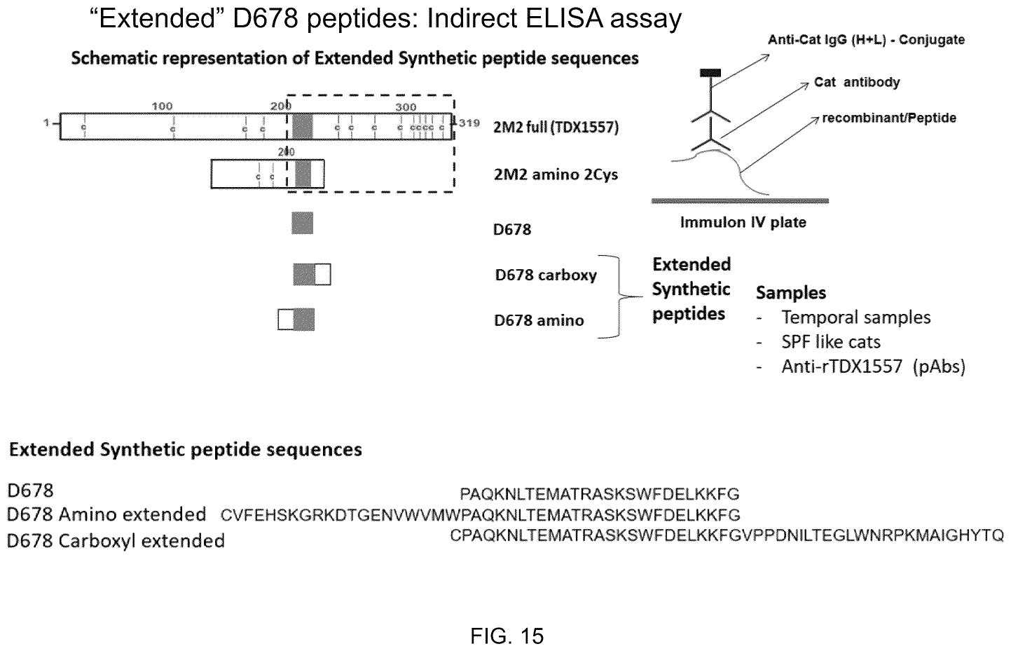

In this example, two extended versions of the D678 peptide were constructed where the immunogenic regions were extended at the amino end (D678 Amino) or the carboxyl end (D678 Carboxyl) and were evaluated via indirect ELISA assay to determine whether either or both extended D678 peptides would exhibit enhanced immunoreactivity relative to a modified D678 peptide. A alignment of the amino acid sequences of modified D678 [SEQ ID NO:78], D678 amino [SEQ ID NO:79], and D678 carboxy peptides [SEQ ID NO:80] is shown in FIG. 15. The overlapping portions of the amino acid of the modified D678 peptide and the extended versions of this peptide (D678 amino and D678 carboxy peptides) are underlined. The D678, D678 amino and D678 carboxy peptides were synthesized by New England Peptide (Gardner, MA).

Modified D678:

(SEQ ID NO: 78)
PAQKNLTEMATRASKSWEDELKKFG

D678 Amino:

(SEQ ID NO: 79)
CVFEHSKGRKDTGENVWVMWPAQKNLTEMATRASKSWFDELKKFG

D678 Carboxy:

(SEQ ID NO: 80)
CPAQKNLTEMATRASKSWFDELKKFGVPPDNILTEGLWNRPKMAIGHYTQ.

N-terminal C residue in SEQ ID NO:80 was added for ease of conjugation, i.e., it is not part of the native sequence.

The indirect ELISA procedure utilized in this Example was described above. Briefly, the recombinant modified D678, D678 amino, D678 carboxy peptides were each coated on Immulon IV plated for an overnight period at 4 C, and the plates were blocked with blocking solution (1% BSA in 100 mM Tris pH 7.5). The plates were washed with 3×PBST, and 100 uL serum samples (1:100 diluted samples) from infected felines were diluted with PRRS diluent (IDEXX PRRS X3 Ab Test, Cat. No. 99-18070, IDEXX Laboratories, Inc., Westbrook, ME, USA) and added to the plates, and the plates were kept for an hour at RT. The plates were washed with 1×PBS-Tween buffer, aspirated and 100 uL/well of conjugate (1:3000 dilution with Enzyme conjugate diluent. The conjugate (ANTI-CAT IGG H+L) was added to the plates and incubated for 30 minutes, RT. The plates were washed, and 50 uL/well of TMB substrate was added and incubated for 5 minutes, RT. 50 uL/well of Stop solution was added. The wells were read at 450 nm. The results are shown in FIGS. 16A to 16F.

FIG. 16A provides results of the evaluation of extended D678 peptides in an indirect ELISA format using temporal, rabbit pAbs and SPF cats. The synthetic peptides D678 amino and D678 carboxyl show similar reactivity to the samples from temporal samples, suggesting that the extended region did not increase the peptide's immunogenicity. While rabbit pAbs against TDX1557 did not show any antibody response to D678 peptide, the rabbit pAbs did react to both the extended D678 amino and D678 carboxyl peptides. D678, D678 amino and D678 carboxyl peptides did not show any significant reactivity to serum samples from experimentally infected cats.

### Example 11: Detection of Lungworm Antigen (2M2 Antigen) in Fecal Samples Using Rabbit Polyclonal Antibodies Against TDX1557

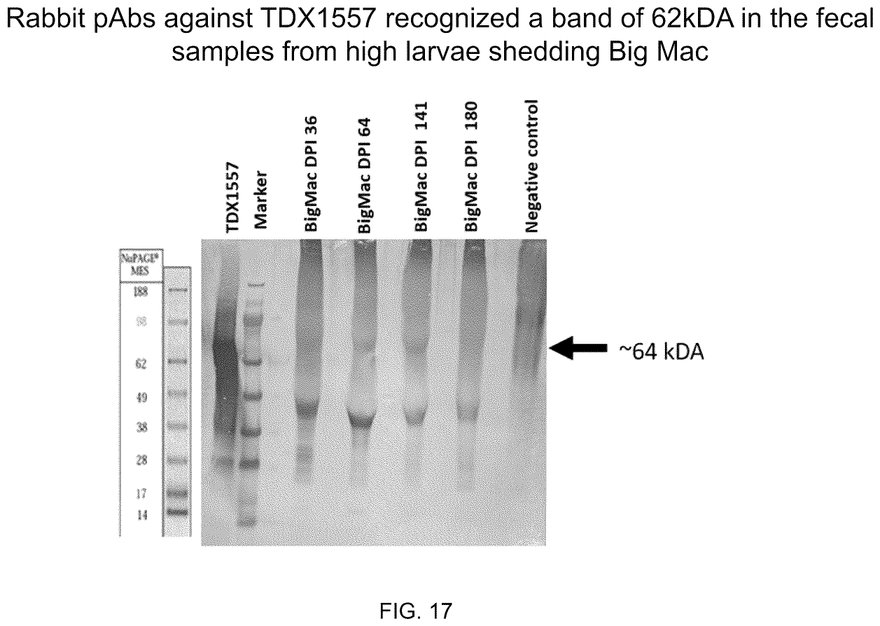

In this example, the fecal samples collected on DPI 36, DPI 64, DPI 141, DPI 180 from the experimentally infected cat (Big Mac) were mixed with SNAP® Giardia diluent (IDEXX Laboratories, Westbrook, ME) at a ratio of 10% (0.5 g of sample in 5 ml of diluent). The suspension was mixed and centrifuged at 10,000 g for 5 minutes. To 100 ul of supernatant, 100 ul of 2× SDS-PAGE sample buffer was added, and the mixture was kept in a heating block (90° C.) for 10 minutes. The mixture was subjected to SDS-PAGE electrophoresis using conventional procedures. The resulting gel was subjected to immunoblot analysis by transferring total proteins of the SDS-PAGE to the nitrocellulose (NC) membrane. The NC membrane was probed with rabbit polyclonal antibodies against TDX1557, followed by the addition of alkaline phosphatase conjugates (Anti-rabbit IgG H+L coupled with alkaline phosphatase. Sigma, St. Louis. MO). The blot was developed with BCIP/NBT substrate (Sigma. St. Louis, MO). The positive control is rTDX1557. The negative control is a fecal sample from a healthy, non-infected cat. FIG. 17 shows the detection of A. abstrusus antigen (2M2) in the fecal samples of an experimentally infected cat, BigMac. The results confirm the detection of a 62 kDA band in the fecal samples of BigMac from DPI 36, DPI 64, DPI 141 using rabbit polyclonal antibodies against TDX1557. The band was not detectable at DPI 180. Although the experimentally infected cat was treated with the anti-parasitic drug on DPI 90, the 2M2 antigen was detectable in the fecal samples until DPI 140. However, 2M2 antigen completely disappeared by DPI 180. The positive control was recombinant 2M2 antigen (rTDX1557), and the negative control was a fecal sample from a healthy, non-infected cat.

### Example 12: Alanine Scanning of an 2M2 Epitope

This example shows the effect of amino acid substitutions on antibody binding in the region defined by peptides D6, D7 and D8. To this end, an alanine scan was performed. As shown in the tables below, alanine (bold lettering) was substituted for native amino acids in the peptide D7M (SEQ ID NO:90) and in peptide D8M (SEQ ID NO:106). At positions where alanine was already present in the native sequence, glycine was substituted.

TABLE: D7M peptides

| SEQ ID NO: | Peptide ID | Peptide Sequence |
|---|---|---|
| 90 | D7M0 | LTEMATRASKSWFDE |
| 91 | D7M1 | ATEMATRASKSWEDE |
| 92 | D7M2 | LAEMATRASKSWFDE |
| 93 | D7M3 | LTAMATRASKSWFDE |
| 94 | D7M4 | LTEAATRASKSWFDE |
| 95 | D7M5 | LTEMGTRASKSWFDE |
| 96 | D7M6 | LTEMAARASKSWFDE |
| 97 | D7M7 | LTEMATAASKSWEDE |
| 98 | D7M8 | LTEMATRGSKSWFDE |
| 99 | D7M9 | LTEMATRAAKSWFDE |
| 100 | D7M10 | LTEMATRASASWFDE |
| 101 | D7M11 | LTEMATRASKAWFDE |
| 102 | D7M12 | LTEMATRASKSAFDE |
| 103 | D7M13 | LTEMATRASKSWADE |
| 104 | D7M14 | LTEMATRASKSWFAE |
| 105 | D7M15 | LTEMATRASKSWFDA |

TABLE: D8M peptides

| SEQ ID NO: | Peptide ID | Peptide Sequence |
|---|---|---|
| 106 | D8M0 | TRASKSWFDELKKFG |
| 107 | D8M1 | ARASKSWFDELKKFG |
| 108 | D8M2 | TAASKSWFDELKKFG |
| 109 | D8M3 | TRGSKSWFDELKKFG |
| 110 | D8M4 | TRAAKSWFDELKKFG |
| 111 | D8M5 | TRASASWFDELKKFG |
| 112 | D8M6 | TRASKAWFDELKKFG |
| 113 | D8M7 | TRASKSAFDELKKFG |
| 114 | D8M8 | TRASKSWADELKKFG |
| 115 | D8M9 | TRASKSWFAELKKFG |
| 116 | D8M10 | TRASKSWFDALKKFG |

The peptides were synthesized with and labeled with biotin at the N-termini using standard procedure.

An amino acid critical for antibody binding can be detected if a large drop in the OD value is observed indicating that the substituted amino acid was necessary for proper antibody binding. In addition, this experiment provides the opportunity for the identification of advantageous substitutions if the OD the value is increased compared to the original peptide.

An indirect ELISA assay was performed as described in Example 7 A, testing the immunoreactivity of peptides D7M0-D7M15 and D8M0-D8M10 with pooled sera from three cats naturally infected with A. abstrusus (i.e., field positives). The assays were run in triplicate. The OD values were normalized against the immunoreactivity (OD values) with serum from a Day 0 sample of an experimentally infected cat.

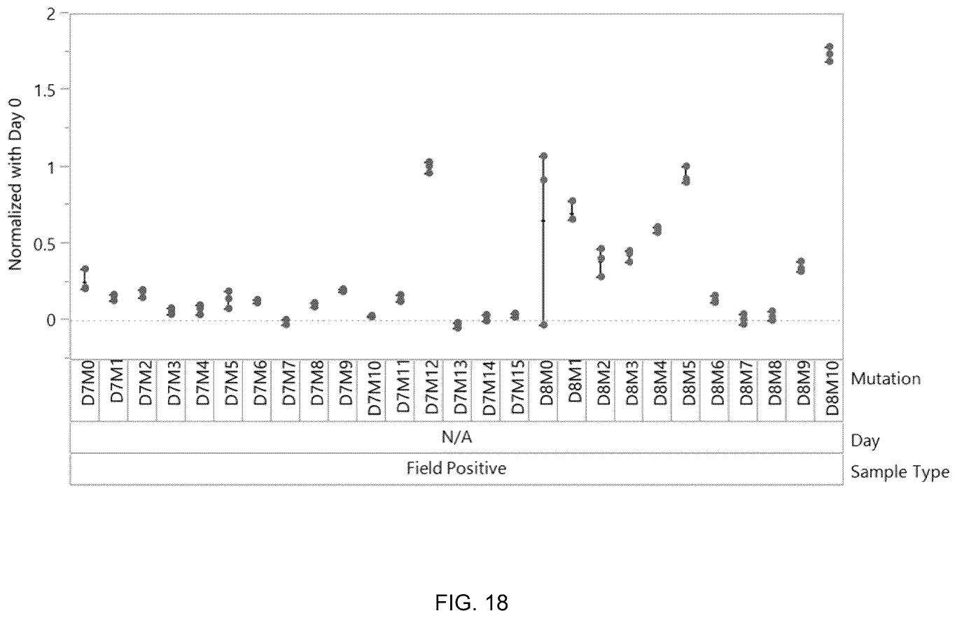

The results are shown in FIG. 18. As compared to D8M0, the OD values were strongly reduced with D8M7 (tryptophan to alanine) and D8M8 (phenylalanine to alanine), indicating that amino acid positions 7 and 8 are critical for binding of antibodies in sera from cats infected with A. abstrusus. As compared to DSM0, the OD values were increased with D8M10, indicating the substitution ofglutamic acid (a negatively charged amino acid) to alanine (a small non-polar amino acid) enhanced the binding of the peptide with antibodies in sera from cats infected with A. abstrusus. Similar results were obtained when the peptides were tested with sera from various time points of cats experimentally infected with A. abstrusus (data not shown).

Therefore, the amino acids corresponding to position 7 (tryptophan) and position 8 (phenylalanine) of peptide D8M0 (SEQ ID NO:106) are critical for binding of antibodies from cats infected with A. abstrusus. These amino acid positions correspond to position 4 (tryptophan) and position 5 (phenylalanine) of the peptide having the sequence SKSWF (SEQ ID NO:81).

This data demonstrate that the consensus sequence of the core epitope is XXXWF (SEQ ID NO:117). Thus, in one embodiment of the disclosure, the polypeptide comprises XXXWF (SEQ ID NO:117), wherein (a) amino acids W and F at positions 4 and 5 respectively are retained and at least one X at positions 1, 2 and 3 is substituted with a conservative or non-conservative amino acid; (b) amino acids W and F at positions 4 and 5 respectively are retained and conservative amino acid substitutions of X are made at positions 1, 2 and 3; (c) amino acids W and F at positions 4 and 5 respectively are retained, the X at positions 1 and 3 are independently amino acid S, a conservative amino acid or a non-conservative amino acid, and the X at position 2 is amino acid K, a conservative amino acid or a non-conservative amino acid; and (d) amino acid W at position 4 or amino acid F at position 5 is substituted with a conservative amino acid, and wherein the X at positions 1 and 3 are independently amino acid S, a conservative amino acid or a non-conservative amino acid, and the X at position 2 is K, a conservative amino acid or a non-conservative amino acid.

Some embodiments of the present disclosure include:

Embodiment 1: A polypeptide comprising the amino acid sequence SEQ ID NO:2 (TDX1557), SEQ ID NO:3 (rTDX1557), SEQ ID NO:4 (T2 truncate), SEQ ID NO:5 (T3 truncate), SEQ ID NO:6 (T4 truncate), SEQ ID NO:7 (C10 peptide), SEQ ID NO:8 (C11 peptide), SEQ ID NO: 9 (C12 peptide), SEQ ID NO:10 (D1 peptide), SEQ ID NO:1l (D2 peptide), SEQ ID NO:12 (D3 peptide), SEQ ID NO:13 (D4 peptide), SEQ ID NO:14 (D5 peptide), SEQ ID NO:15 (D6 peptide), SEQ ID NO:16 (D7 peptide), SEQ ID NO:17 (D8 peptide), SEQ ID NO:18 (d678 combo), SEQ ID NO:78 (modified d678), SEQ ID NO:79 (d678-amino extended). SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106 (D8M0), SEQ ID NO:107 (D8M1 peptide), SEQ ID NO:108 (D8M2), SEQ ID NO:109 (D8M3), SEQ ID NO:110 (D8M4), SEQ ID NO:11 (D8M5), SEQ ID NO:112 (D8M6), SEQ ID NO:115 (D8M9), SEQ ID NO:116 (D8M10), or a polypeptide comprising XXXWF (SEQ ID NO:117), wherein (a) amino acids W and F at positions 4 and 5 respectively are retained and at least one X at positions 1, 2 and 3 is substituted with a conservative or non-conservative amino acid; (b) amino acids W and F at positions 4 and 5 respectively are retained and conservative amino acid substitutions of X are made at positions 1, 2 and 3; (c) amino acids W and F at positions 4 and 5 respectively are retained, the X at positions 1 and 3 are independently amino acid S, a conservative amino acid or a non-conservative amino acid, and the X at position 2 is amino acid K, a conservative amino acid or a non-conservative amino acid; and (d) amino acid W at position 4 or amino acid F at position 5 is substituted with a conservative amino acid, and wherein the X at positions 1 and 3 are independently amino acid S, a conservative amino acid or a non-conservative amino acid, and the X at position 2 is K, a conservative amino acid or a non-conservative amino acid.

Embodiment 2: The polypeptide according to Embodiment 1, wherein the polypeptide is selected from the group consisting of the amino acid sequence SEQ ID NO:2 (TDX1557), SEQ ID NO:3 (rTDX1557), SEQ ID NO:4 (T2 truncate), SEQ ID NO:5 (T3 truncate), SEQ ID NO:6 (T4 truncate), SEQ ID NO:7 (C10 peptide), SEQ ID NO:8 (C11 peptide), SEQ ID NO: 9 (C12 peptide), SEQ ID NO:10 (D1 peptide), SEQ ID NO:11 (D2 peptide), SEQ ID NO:12 (D3 peptide), SEQ ID NO:13 (D4 peptide), SEQ ID NO:14 (D5 peptide), SEQ ID NO:15 (D6 peptide), SEQ ID NO:16 (D7 peptide), SEQ ID NO:17 (D8 peptide), SEQ ID NO:18 (d678 combo), SEQ ID NO:78 (modified d678), SEQ ID NO:79 (d678-amino extended), SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO: 105, SEQ ID NO:106 (D8M0), SEQ ID NO:107 (D8M1 peptide), SEQ ID NO:108 (D8M2), SEQ ID NO:109 (D8M3), SEQ ID NO:110 (D8M4), SEQ ID NO:111 (D8M5), SEQ ID NO:112 (D8M6), SEQ ID NO:115 (D8M9), SEQ ID NO:116 (D8M10), or a polypeptide comprising XXXWF (SEQ ID NO:117), wherein (a) amino acids W and F at positions 4 and 5 respectively are retained and at least one X at positions 1, 2 and 3 is substituted with a conservative or non-conservative amino acid; (b) amino acids W and F at positions 4 and 5 respectively are retained and conservative amino acid substitutions of X are made at positions 1, 2 and 3; (c) amino acids W and F at positions 4 and 5 respectively are retained, the X at positions 1 and 3 are independently amino acid S, a conservative amino acid or a non-conservative amino acid, and the X at position 2 is amino acid K, a conservative amino acid or a non-conservative amino acid; and (d) amino acid W at position 4 or amino acid F at position 5 is substituted with a conservative amino acid, and wherein the X at positions 1 and 3 are independently amino acid S, a conservative amino acid or a non-conservative amino acid, and the X at position 2 is K, a conservative amino acid or a non-conservative amino acid.

Embodiment 3: The polypeptide of Embodiment 1, wherein the polypeptide includes at least 5 contiguous amino acids of the amino acid sequence SEQ ID NO:2 (TDX1557), SEQ ID NO:3 (rTDX1557), SEQ ID NO:4 (T2 truncate), SEQ ID NO:5 (T3 truncate), SEQ ID NO:6 (T4 truncate), SEQ ID NO:7 (C10 peptide), SEQ ID NO:8 (C11 peptide), SEQ ID NO: 9 (C12 peptide), SEQ ID NO:10 (D1 peptide), SEQ ID NO:11 (D2 peptide), SEQ ID NO:12 (D3 peptide), SEQ ID NO:13 (D4 peptide), SEQ ID NO:14 (D5 peptide), SEQ ID NO:15 (D6 peptide), SEQ ID NO:16 (D7 peptide), SEQ ID NO:17 (D8 peptide), SEQ ID NO:18 (d678 combo), SEQ ID NO:78 (modified d678), SEQ ID NO:79 (d678-amino extended), SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106 (D8M0), SEQ ID NO:107 (D8M1 peptide), SEQ ID NO:108 (D8M2), SEQ ID NO:109 (D8M3), SEQ ID NO:110 (D8M4), SEQ ID NO:111 (D8M5), SEQ ID NO: 112 (D8M6), SEQ ID NO:115 (D8M9), SEQ ID NO:116 (D8M10), or a polypeptide comprising XXXWF (SEQ ID NO:117), wherein (a) amino acids W and F at positions 4 and 5 respectively are retained and at least one X at positions 1, 2 and 3 is substituted with a conservative or non-conservative amino acid; (b) amino acids W and F at positions 4 and 5 respectively are retained and conservative amino acid substitutions of X are made at positions 1, 2 and 3; (c) amino acids W and F at positions 4 and 5 respectively are retained, the X at positions 1 and 3 are independently amino acid S, a conservative amino acid or a non-conservative amino acid, and the X at position 2 is amino acid K, a conservative amino acid or a non-conservative amino acid; and (d) amino acid W at position 4 or amino acid F at position 5 is substituted with a conservative amino acid, and wherein the X at positions 1 and 3 are independently amino acid S, a conservative amino acid or a non-conservative amino acid, and the X at position 2 is K, a conservative amino acid or a non-conservative amino acid.

Embodiment 4: The polypeptide of Embodiment 1, wherein the polypeptide is at least 5, 6, 7, 8, 9, 10, 15, 20, 25, 30, 35, 40, 45, 50, 55, 60, 65, 70, 75, 80, 85, 90, 95, 100, or full size of TDX1557 amino acids. Alternatively, the polypeptide of Embodiment 1, wherein the polypeptide comprises 5, 6, 7, 8, 9, 10, 11, 12, 13, 14, 15, or more contiguous amino acids as set forth in SEQ ID NO:2-18 and 78-117.

Embodiment 5: The polypeptide of Embodiment 1, wherein the polypeptide has 10 to 25 linked amino acids and comprises at least 5, 6, 7, 8, 9 or 10 contiguous amino acids as set forth in SEQ ID NO:2 (TDX1557), SEQ ID NO:3 (rTDX1557), SEQ ID NO:4 (T2 truncate), SEQ ID NO:5 (T3 truncate), SEQ ID NO:6 (T4 truncate). SEQ ID NO:7 (C10 peptide), SEQ ID NO:8 (C11 peptide), SEQ ID NO: 9 (C12 peptide), SEQ ID NO:10 (D1 peptide), SEQ ID NO:11 (D2 peptide), SEQ ID NO:12 (D3 peptide), SEQ ID NO:13 (D4 peptide), SEQ ID NO:14 (D5 peptide), SEQ ID NO:15 (D6 peptide), SEQ ID NO:16 (D7 peptide), SEQ ID NO:17 (D8 peptide), SEQ ID NO:18 (d678 combo), SEQ ID NO:78 (modified d678), SEQ ID NO:79 (d678-amino extended), SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106 (D8M0), SEQ ID NO:107 (D8M1 peptide), SEQ ID NO:108 (D8M2), SEQ ID NO:109 (D8M3), SEQ ID NO:110 (D8M4), SEQ ID NO:111 (D8M5), SEQ ID NO:112 (D8M6), SEQ ID NO:115 (D8M9), SEQ ID NO:116 (D8M10), or a polypeptide comprising XXXWF (SEQ ID NO:117), wherein (a) amino acids W and F at positions 4 and 5 respectively are retained and at least one X at positions 1, 2 and 3 is substituted with a conservative or non-conservative amino acid; (b) amino acids W and F at positions 4 and 5 respectively are retained and conservative amino acid substitutions of X are made at positions 1, 2 and 3; (c) amino acids W and F at positions 4 and 5 respectively are retained, the X at positions 1 and 3 are independently amino acid S, a conservative amino acid or a non-conservative amino acid, and the X at position 2 is amino acid K, a conservative amino acid or a non-conservative amino acid; and (d) amino acid W at position 4 or amino acid F at position 5 is substituted with a conservative amino acid, and wherein the X at positions 1 and 3 are independently amino acid S, a conservative amino acid or a non-conservative amino acid, and the X at position 2 is K, a conservative amino acid or a non-conservative amino acid.

Embodiment 6: The polypeptide of Embodiment 1, wherein the peptide is labeled or attached to a solid support.

Embodiment 7: A method of detecting the presence or absence of one or more feline lungworm antibodies in a biological sample from a mammal, the method comprising:
(a) contacting the sample with one or more polypeptides of any one of Embodiments 1-6 capable of specifically binding to one or more feline lungworm antibodies;
(b) forming antibody-polypeptide complexes in the presence of the one or more antibodies, if any, in the sample; and
(c) detecting the presence or absence of the antibody-polypeptide complexes, if any.

Embodiment 8: A method of diagnosing whether a mammal is infected with one or more feline lungworms, the method comprising the steps of:
(a) contacting a biological sample from the mammal with one or more polypeptides of any one of Embodiments 1-6 capable of specifically binding to one or more feline lungworm antibodies;
(b) forming antibody-polypeptide complexes in the presence of the one or more antibodies, if any, in the sample;
(c) detecting the presence or absence of the antibody-polypeptide complexes, if any; and
(d) diagnosing the mammal as having feline lungworm if the antibody-polypeptide complexes are present.

Embodiment 9: A method of diagnosing and treating a mammal infected with one or more feline lungworms, the method comprising the steps of:
(a) contacting a biological sample from a feline with one or more polypeptides of any one of Embodiments 1-6 capable of specifically binding to one or more feline lungworm antibodies;
(b) forming antibody-polypeptide complexes in the presence of the one or more antibodies, if any, in the sample;
(c) detecting the presence or absence of the antibody-polypeptide complexes, if any;
(d) diagnosing the mammal as having feline lungworm if antibody-polypeptide complexes are present; and
(e) administering an effective amount of one or more therapeutic agents to treat the mammal having feline lungworm infection.

Embodiment 10: A method of detecting the presence or absence of one or more feline lungworm antigens in a biological sample, the method comprising:
(a) contacting the sample from a feline with one or more antibodies that specifically bind to one or more polypeptides of any one of Embodiments 1-6;
(b) forming antibody-antigen complexes in the presence of the one or more feline lungworm antigens, if any, in the sample; and
(c) detecting the presence or absence of the antibody-antigen complexes, if any.

Embodiment 11: A method of diagnosing whether a mammal is infected with one or more feline lungworms, the method comprising the steps of:
(a) contacting a biological sample from the mammal with one or more antibodies that specifically bind to one or more polypeptides of any one of Embodiments 1-6;
(b) forming antibody-antigen complexes in the presence of one or more feline lungworm antigens, if any, in the sample;
(c) detecting the presence or absence of the antibody-antigen complexes, if any; and
(d) diagnosing the mammal as having feline lungworm if antibody-antigen complexes are present.

Embodiment 12: A method of diagnosing and treating a mammal infected with one or more feline lungworms, the method comprising the steps of:
(a) contacting a biological sample from a mammal with one or more antibodies that specifically bind to one or more polypeptides of any one of Embodiments 1-6;
(b) forming antibody-antigen complexes in the presence of one or more feline lungworm antigens, if any, in the sample;
(c) detecting the presence or absence of the antibody-antigen complexes, if any;
(d) diagnosing the mammal as having feline lungworm if antibody-antigen complexes are present; and
(e) administering an effective amount of one or more therapeutic agents to treat the mammal having feline lungworm infection.

Embodiment 13: The method of any one of Embodiments 9 or 12, wherein step (e) further includes one or more additional therapeutic agents to: (a) treat infection by one or more helminthic worm parasites, one or more non-worm parasites, one or more viruses, one or more fungi, one or more protozoa, or one or more bacteria.

Embodiment 14: The method of any one of Embodiments 8, 9, 11, or 12, wherein step (d) further includes one or more therapeutic agents to (b) control, repel or kill an intermediate host of feline lungworm parasite, a platyhelminthic worm parasite, helminthic worm parasite, non-worm parasite, virus, fungus, protozoa, or bacterium.

Embodiment 15: The method according to any one of Embodiments 1-14, wherein the one or more antibodies bind to Aelurostrongylus abstrusus but not to an antibody to Capillaria aerophila or Troglostrongylus brevior.

Embodiment 16: The method according to Embodiments 7-9, wherein the one or more polypeptides do not bind to an antibody to roundworm, tapeworm, whipworm, or hookworm.

Embodiment 17: The method according to Embodiments 10-12, wherein the one or more antibodies do not bind to an antigen of roundworm, tapeworm, whipworm, or hookworm.

Embodiment 18: The method of any one of Embodiments 16 or 17, wherein the roundworm is Toxocara.

Embodiment 19: The method of any one of Embodiments 16 to 18, wherein the tapeworm is Taenia or Diphyidium.

Embodiment 20: The method of any one of Embodiments 16 to 19, wherein the bookworm is Ancylostoma.

Embodiment 21: The method of any one of Embodiments 7-20, wherein step (c) of detecting the presence or absence of the complexes further includes the step of providing at least one secondary antibody that binds to the one or more complexes.

Embodiment 22: The method of Embodiment 21, wherein the secondary antibody is labeled or attached to a solid support.

Embodiment 23: The method of Embodiment 22, wherein the solid support forms part of an enzyme-linked immunosorbent assay device.

Embodiment 24: The method of Embodiment 23, wherein the enzyme-linked immunosorbent assay device is a lateral flow immunoassay device.

Embodiment 25: The method of any one of Embodiments 1-24, wherein the mammal is a feline.

Embodiment 26: A polynucleotide encoding a polypeptide of any one of Embodiments 1-6.

Embodiment 27: The polynucleotide of Embodiment 26, wherein the polynucleotide is an RNA.

Embodiment 28: The polynucleotide of Embodiment 27, wherein the polynucleotide comprises one or more modified uridine residues.

Embodiment 29: The polynucleotide of Embodiment 28, wherein the modified uridine residues is pseudouridine or methylpseudouridine.

Embodiment 30: The polynucleotide of Embodiment 26, wherein the polynucleotide is an mRNA.

Embodiment 31: The polynucleotide of Embodiment 30, wherein the polynucleotide is contained within a lipid nanoparticle.

Embodiment 32: A vaccine composition comprising one or more polynucleotides of any one of Embodiments 26 to 31 and a pharmaceutically acceptable carrier.

Embodiments 33: A vaccine composition comprising one or more polypeptides of any one of Embodiments 1-6 and a pharmaceutically acceptable carrier.

Embodiment 34: An immunocomplex comprising one or more polypeptides of any one of Embodiments 1-6 and one or more feline lungworm antibodies specifically bound to the one or more polypeptides.

Embodiment 35: An immunocomplex comprising one or more antibodies that specifically binds to one or more polypeptides of any one of Embodiments 1-6 and one or more feline lungworm antigens specifically bound to the one or more antibodies.

Embodiment 36: A device for detecting the presence or absence of one or more feline lungworm antibodies from a biological sample, the device comprising a solid support, wherein the solid support has immobilized thereon one or more polypeptides of any one of Embodiments 1-6.

Embodiment 37: The device of Embodiment 36, wherein the one or more polypeptides do not specifically cross-react with one or more antibodies to roundworm, tapeworm, hookworm, Capillaria aerophila or Troglostrongylus brevior.

Embodiment 38: The device of Embodiment 37, wherein the roundworm is Toxocara, and/or the tapeworm is Taenia or Diphylidium and/or the hookworm is Ancylostoma.

Embodiment 39: A device for detecting the presence or absence of one or more feline lungworm antigens from a biological sample, the device comprising a solid support, wherein the solid support has immobilized thereon one or more antibodies that specifically bind to one or more polypeptides of any one of Embodiments 1-6.

Embodiment 40: The device of Embodiment 39, wherein the one or more antibodies do not specifically cross-react with one or more antigens of roundworm, tapeworm, hookworm, Capillaria aerophila or Troglostrongylus brevior.

Embodiment 41: The device of Embodiment 40, wherein the roundworm is Toxocara and or the tapeworm is Taenia or Diphylidium and/or the hookworm is Ancylostoma.

Embodiment 42: A method of immunizing a mammal, e.g., feline, against feline lungworm comprising administering the vaccine composition of Embodiment 32 or 33.

1. A method of detecting the presence or absence of one or more feline lungworm antibodies in a biological sample from a mammal, the method comprising:
(a) contacting the sample with one or more polypeptides of claim 18 capable of specifically binding to one or more feline lungworm antibodies;
(b) forming antibody-polypeptide complexes in the presence of the one or more antibodies, if any, in the sample; and
(c) detecting the presence or absence of the antibody-polypeptide complexes, if any.

2. The method of claim 1, further comprising diagnosing whether a mammal is infected with one or more feline lungworms, the method further comprising the step of:
(d) diagnosing the mammal as having feline lungworm if the antibody-polypeptide complexes are present.

3. The method of claim 1, further comprising diagnosing and treating a mammal infected with one or more feline lungworms, the method further comprising the steps of:
(d) diagnosing the mammal as having feline lungworm if antibody-polypeptide complexes are present; and
(e) administering an effective amount of one or more therapeutic agents to treat the mammal having feline lungworm infection.

4. A method of detecting the presence or absence of one or more feline lungworm antigens in a biological sample, the method comprising:
(a) contacting the sample from a feline with one or more antibodies that specifically bind to one or more polypeptides of claim 18;
(b) forming antibody-antigen complexes in the presence of the one or more feline lungworm antigens, if any, in the sample; and
(c) detecting the presence or absence of the antibody-antigen complexes, if any.

5. The method of claim 4, further comprising diagnosing whether a mammal is infected with one or more feline lungworms, the method comprising the step of:
(d) diagnosing the mammal as having feline lungworm if antibody-antigen complexes are present.

6. The method of claim 4, further comprising diagnosing and treating a mammal infected with one or more feline lungworms, the method further comprising the steps of:
(d) diagnosing the mammal as having feline lungworm if antibody-antigen complexes are present; and
(e) administering an effective amount of one or more therapeutic agents to treat the mammal having feline lungworm infection.

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. A polypeptide comprising the amino acid sequence SEQ ID NO:2 (TDX1557), SEQ ID NO:3 (rTDX1557), SEQ ID NO:4 (T2 truncate), SEQ ID NO:5 (T3 truncate), SEQ ID NO:6 (T4 truncate), SEQ ID NO:7 (C10 peptide), SEQ ID NO:8 (C11 peptide), SEQ ID NO: 9 (C12 peptide), SEQ ID NO:10 (D1 peptide), SEQ ID NO:11 (D2 peptide), SEQ ID NO:12 (D3 peptide), SEQ ID NO:13 (D4 peptide), SEQ ID NO:14 (D5 peptide), SEQ ID NO:15 (06 peptide), SEQ ID NO:16 (D7 peptide), SEQ ID NO:17 (D8 peptide), SEQ ID NO:18 (d678 combo), SEQ ID NO:78 (modified d678), SEQ ID NO:79 (d678-amino extended), SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106 (D8M0), SEQ ID NO:107 (D8M1 peptide), SEQ ID NO:108 (D8M2), SEQ ID NO:109 (D8M3), SEQ ID NO:110 (D8M4), SEQ ID NO:111 (D8M5), SEQ ID NO:112 (D8M6), SEQ ID NO:115 (D8M9), SEQ ID NO:116 (D8M10), or a polypeptide comprising XXXWF (SEQ ID NO:117), wherein (a) amino acids W and F at positions 4 and 5 respectively are retained and at least one X at positions 1, 2 and 3 is substituted with a conservative or non-conservative amino acid; (b) amino acids W and F at positions 4 and 5 respectively are retained and conservative amino acid substitutions of X are made at positions 1, 2 and 3; (c) amino acids W and F at positions 4 and 5 respectively are retained, the X at positions 1 and 3 are independently amino acid S, a conservative amino acid or a non-conservative amino acid, and the X at position 2 is amino acid K, a conservative amino acid or a non-conservative amino acid; and (d) amino acid W at position 4 or amino acid F at position 5 is substituted with a conservative amino acid, and wherein the X at positions 1 and 3 are independently amino acid S, a conservative amino acid or a non-conservative amino acid, and the X at position 2 is K, a conservative amino acid or a non-conservative amino acid.

19. (canceled)

20. (canceled)

21. The polypeptide of claim 18, wherein the polypeptide is at least 5, 6, 7, 8, 9, 10, 15, 20, 25, 30, 35, 40, 45, 50, 55, 60, 65, 70, 75, 80, 85, 90, 95, 100, or full size of TDX1557 amino acids.

22. The polypeptide of claim 18, wherein the polypeptide comprises 5, 6, 7, 8, 9, 10, 11, 12, 13, 14, 15, or more contiguous amino acids as set forth in SEQ ID NO:2-18 and 78-117.

23. The polypeptide of claim 20, wherein the polypeptide has 10 to 25 linked amino acids and comprises at least 5, 6, 7, 8, 9 or 10 contiguous amino acids as set forth in SEQ ID NO:2 (TDX1557), SEQ ID NO:3 (rTDX1557), SEQ ID NO:4 (T2 truncate), SEQ ID NO:5 (T3 truncate), SEQ ID NO:6 (T4 truncate), SEQ ID NO:7 (C10 peptide), SEQ ID NO:8 (C11 peptide), SEQ ID NO: 9 (C12 peptide), SEQ ID NO:10 (D1 peptide), SEQ ID NO:11 (D2 peptide), SEQ ID NO:12 (D3 peptide), SEQ ID NO:13 (D4 peptide), SEQ ID NO:14 (D5 peptide), SEQ ID NO:15 (D6 peptide), SEQ ID NO:16 (D7 peptide), SEQ ID NO:17 (D8 peptide), SEQ ID NO:18 (d678 combo), SEQ ID NO:78 (modified d678), SEQ ID NO:79 (d678-amino extended), SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106 (D8M0 peptide), SEQ ID NO:107 (D8M1 peptide), SEQ ID NO:108 (D8M2), SEQ ID NO:109 (D8M3), SEQ ID NO:110 (D8M4), SEQ ID NO:111 (D8M5), SEQ ID NO:112 (D8M6), SEQ ID NO:115 (D8M9), SEQ ID NO:116 (D8M10), or a polypeptide comprising XXXWF (SEQ ID NO:117), wherein (a) amino acids W and F at positions 4 and 5 respectively are retained and at least one X at positions 1, 2 and 3 is substituted with a conservative or non-conservative amino acid; (b) amino acids W and F at positions 4 and 5 respectively are retained and conservative amino acid substitutions of X are made at positions 1, 2 and 3; (c) amino acids W and F at positions 4 and 5 respectively are retained, the X at positions 1 and 3 are independently amino acid S, a conservative amino acid or a non-conservative amino acid, and the X at position 2 is amino acid K, a conservative amino acid or a non-conservative amino acid; and (d) amino acid W at position 4 or amino acid F at position 5 is substituted with a conservative amino acid, and wherein the X at positions 1 and 3 are independently amino acid S, a conservative amino acid or a non-conservative amino acid, and the X at position 2 is K, a conservative amino acid or a non-conservative amino acid.

24. (canceled)

25. A polynucleotide encoding a polypeptide of claim 18.

26. (canceled)

27. (canceled)

28. (canceled)

29. (canceled)

30. (canceled)

31. A vaccine composition comprising one or more polynucleotides of claim 25 and a pharmaceutically acceptable carrier.

32. A vaccine composition comprising one or more polypeptides of claim 18 and a pharmaceutically acceptable carrier.

33. An immunocomplex comprising one or more polypeptides of claim 18 and one or more feline lungworm antibodies specifically bound to the one or more polypeptides.

34. An immunocomplex comprising one or more antibodies that specifically binds to one or more polypeptides of claim 18 and one or more feline lungworm antigens specifically bound to the one or more antibodies.

35. A device for detecting the presence or absence of one or more feline lungworm antibodies from a biological sample, the device comprising a solid support, wherein the solid support has immobilized thereon one or more polypeptides of claim 18.

36. (canceled)

37. (canceled)

38. A device for detecting the presence or absence of one or more feline lungworm antigens from a biological sample, the device comprising a solid support, wherein the solid support has immobilized thereon one or more antibodies that specifically bind to one or more polypeptides of claim 18.

39. (canceled)

40. (canceled)

41. A kit comprising the device of claim 35.

42. A method of immunizing a mammal against feline lungworm comprising administering the vaccine composition of claim 31.

43. A kit comprising the device of claim 38.

44. A method of immunizing a mammal against feline lungworm comprising administering the vaccine composition of claim 32.


SEQUENCE LISTING (124 sequences)

SEQ ID NO: 1 - DNA, 1020 bp, synthetic construct
  misc_feature 1..1020: note = Synthetic construct
  source 1..1020: mol_type = other DNA
atgtcggccatagtgttaactattgcattcacattgtttctattcggcggattccgtgccaacgatgagatggacgagaacaatcctgttgcccttccagggaagtcaaacgtcagcgaaccttgccaaacaacgacaagtaatccaatccatccaacgacgggagaacaactcgaaactacgacaggaaaaccacgcagaacaacgaaaggcagaatactcaagacaacgacagtcaaaccgattaaaacaacaaaaggcaaaaagctgaaaacaacaaaaggcaaattactcaagacaacaacagtcaaaccattcaaaacaacaaagggaaaaccactcaaaacgacaactaaggaaccattccaatcaacaacaggcggtccagtgggaccaacaacagacagatgtaagctgaacaacgggatgaatgaccaattgagggatattatgttgaatgaccataataccctaagatcactggcggctaaaggcttggcagaaaatccccttggtactaacggccgcgcaccgaaagcggcgcgaatgctcaaaatggtatatgattgcaatgttgagaaaaccgctatgattcatgcgaaaaaatgcgtattcgaacacagcaagggaagaaaagacactggagaaaacgtctgggtaatgtggccagcacagaaaaatttgaccgagatggctacacgtgccagcaaatcttggttcgatgaattaaaaaaattcggagtgccaccagataacattctcacagagggactctggaatcgcccaaagatggcaatcggccattatactcaaatggtgtgggaaggttcatacaaacttggctgtggcgttgcaacgtgctcggacaaaacacttatcgtctgccaatactcgcctgcaggaaactacataggaagtattatttacgccatcggagagccatgtaaaactgacgaagattgtaagtgcgaaggatgcaaatgtagtcgcgatgaagcactctgcatcaaaccgaactag

SEQ ID NO: 2 - protein, 339 aa, synthetic construct
  REGION 1..339: note = Synthetic peptide
MSAIVLTIAFTLFLFGGFRANDEMDENNPVALPGKSNVSEPCQTTTSNPIHPTTGEQLETTTGKPRRTTKGRILKTTTVKPIKTTKGKKLKTTKGKLLKTTTVKPFKTTKGKPLKTTTKEPFQSTTGGPVGPTTDRCKLNNGMNDQLRDIMLNDHNTLRSLAAKGLAENPLGTNGRAPKAARMLKMVYDCNVEKTAMIHAKKCVFEHSKGRKDTGENVWVMWPAQKNLTEMATRASKSWFDELKKFGVPPDNILTEGLWNRPKMAIGHYTQMVWEGSYKLGCGVATCSDKTLIVCQYSPAGNYIGSIIYAIGEPCKTDEDCKCEGCKCSRDEALCIKPN

SEQ ID NO: 3 - protein, 358 aa, synthetic construct
  REGION 1..358: note = Synthetic peptide
MDWTWRVFFLLALATGVHSENNDEMDENNPVALPGKSNVSEPCQTTTSNPIHPTTGEQLETTTGKPRRTTKGRILKTTTVKPIKTTKGKKLKTTKGKLLKTTTVKPFKTTKGKPLKTTTKEPFQSTTGGPVGPTTDRCKLNNGMNDQLRDIMLNDHNTLRSLAAKGLAENPLGTNGRAPKAARMLKMVYDCNVEKTAMIHAKKCVFEHSKGRKDTGENVWVMWPAQKNLTEMATRASKSWFDELKKFGVPPDNILTEGLWNRPKMAIGHYTQMVWEGSYKLGCGVATCSDKTLIVCQYSPAGNYIGSIIYAIGEPCKTDEDCKCEGCKCSRDEALCIKPNHHHHHHHGVLAPHDSVLQ

SEQ ID NO: 4 - protein, 239 aa, synthetic construct
  REGION 1..239: note = Synthetic peptide
MDWTWRVFFLLALATGVHSENMNDQLRDIMLNDHNTLRSLAAKGLAENPLGTNGRAPKAARMLKMVYDCNVEKTAMIHAKKCVFEHSKGRKDTGENVWVMWPAQKNLTEMATRASKSWFDELKKFGVPPDNILTEGLWNRPKMAIGHYTQMVWEGSYKLGCGVATCSDKTLIVCQYSPAGNYIGSIIYAIGEPCKTDEDCKCEGCKCSRDEALCIKPNAAAHHHHHHHGVLAPHDSVLQ

SEQ ID NO: 5 - protein, 239 aa, synthetic construct
  REGION 1..239: note = Synthetic peptide
MDWTWRVFFLLALATGVHSENNDEMDENNPVALPGKSNVSEPCQTTTSNPIHPTTGEQLETTTGKPRRTTKGRILKTTTVKPIKTTKGKKLKTTKGKLLKTTTVKPFKTTKGKPLKTTTKEPFQSTTGGPVGPTTDRCKLNNGMNDQLRDIMLNDHNTLRSLAAKGLAENPLGTNGRAPKAARMLKMVYDCNVEKTAMIHAKKCVFEHSKGRKDTGEAAAHHHHHHHHGVLAPHDSVLQ

SEQ ID NO: 6 - protein, 172 aa, synthetic construct
  REGION 1..172: note = Synthetic peptide
MDWTWRVFFLLALATGVHSENGRKDTGENVWVMWPAQKNLTEMATRASKSWFDELKKFGVPPDNILTEGLWNRPKMAIGHYTQMVWEGSYKLGCGVATCSDKTLIVCQYSPAGNYIGSIIYAIGEPCKTDEDCKCEGCKCSRDEALCIKPNAAAHHHHHHHGVLAPHDSVLQ

SEQ ID NO: 7 - protein, 15 aa, synthetic construct
  REGION 1..15: note = Synthetic peptide
MVYDCNVEKTAMIHA

SEQ ID NO: 8 - protein, 15 aa, synthetic construct
  REGION 1..15: note = Synthetic peptide
NVEKTAMIHAKKCVF

SEQ ID NO: 9 - protein, 15 aa, synthetic construct
  REGION 1..15: note = Synthetic peptide
AMIHAKKCVFEHSKG

SEQ ID NO: 10 - protein, 15 aa, synthetic construct
  REGION 1..15: note = Synthetic peptide
KKCVFEHSKGRKDTG

SEQ ID NO: 11 - protein, 15 aa, synthetic construct
  REGION 1..15: note = Synthetic peptide
EHSKGRKDTGENVWV

SEQ ID NO: 12 - protein, 15 aa, synthetic construct
  REGION 1..15: note = Synthetic peptide
RKDTGENVWVMWPAQ

SEQ ID NO: 13 - protein, 15 aa, synthetic construct
  REGION 1..15: note = Synthetic peptide
ENVWVMWPAQKNLTE

SEQ ID NO: 14 - protein, 15 aa, synthetic construct
  REGION 1..15: note = Synthetic peptide
MWPAQKNLTEMATRA

SEQ ID NO: 15 - protein, 15 aa, synthetic construct
  REGION 1..15: note = Synthetic peptide
KNLTEMATRASKSWF

SEQ ID NO: 16 - protein, 15 aa, synthetic construct
  REGION 1..15: note = Synthetic peptide
MATRASKSWFDELKK

SEQ ID NO: 17 - protein, 15 aa, synthetic construct
  REGION 1..15: note = Synthetic peptide
SKSWFDELKKFGVPP

SEQ ID NO: 18 - protein, 26 aa, synthetic construct
  REGION 1..26: note = Synthetic peptide
CPAQKNLTEMATRASKSWFDELKKFG

SEQ ID NO: 19 - DNA, 1080 bp, synthetic construct
  misc_feature 1..1080: note = codon optimized sequence for rTDX1557
  source 1..1080: mol_type = other DNA
atggactggacttggagagtgttcttcctgctggccctggctacaggcgtgcacagcgagaacaacgacgagatggacgagaacaatcccgtggctctgcccggcaagagcaatgtgtctgagccttgccagaccaccaccagcaatcctattcaccctaccaccggcgagcagctggaaaccacaacaggcaagcccagacggaccaccaagggcagaatcctgaaaaccaccaccgtgaagcccatcaagacaacaaagggcaagaagctcaagacgaccaaggggaagctgctgaaaacaacaacagtcaaacccttcaagaccacgaagggaaagccccttaagacaaccaccaaagagcccttccagagcaccacaggtggaccagtgggcccaaccaccgatagatgcaagctgaacaacggcatgaacgaccagctgcgggacatcatgctgaacgatcacaacaccctgagaagcctggccgccaaaggactggccgaaaatcctctgggcaccaacggcagagcccctaaagctgccagaatgctgaagatggtgtacgactgcaacgtggaaaagaccgccatgatccacgccaagaaatgcgtgttcgagcacagcaaaggccggaaggataccggcgagaacgtgtgggttatgtggcccgctcagaaaaacctgaccgagatggccaccagagccagcaagagttggttcgacgagctgaagaaattcggcgtgccacctgacaacatcctgaccgaaggcctgtggaacagacccaagatggccattggccactacacccagatggtctgggagggcagctacaagcttggatgtggcgtggccacatgcagcgacaagaccctgatcgtgtgccagtatagccctgccggcaactacatcggcagcatcatctatgccatcggcgagccctgcaagaccgacgaggattgcaagtgcgagggctgcaagtgcagcagagatgaggccctgtgcatcaagcccaatcaccaccatcatcaccatcacggcgtgctggcccctcacgattctgttcttcagtaataa

SEQ ID NO: 20 - DNA, 6471 bp, synthetic construct
  misc_feature 1..6471: note = Synthetic construct
  source 1..6471: mol_type = other DNA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccgccaccatggactggacttggagagtgttcttcctgctggccctggctacaggcgtgcacagcgagaacaacgacgagatggacgagaacaatcccgtggctctgcccggcaagagcaatgtgtctgagccttgccagaccaccaccagcaatcctattcaccctaccaccggcgagcagctggaaaccacaacaggcaagcccagacggaccaccaagggcagaatcctgaaaaccaccaccgtgaagcccatcaagacaacaaagggcaagaagctcaagacgaccaaggggaagctgctgaaaacaacaacagtcaaacccttcaagaccacgaagggaaagccccttaagacaaccaccaaagagcccttccagagcaccacaggtggaccagtgggcccaaccaccgatagatgcaagctgaacaacggcatgaacgaccagctgcgggacatcatgctgaacgatcacaacaccctgagaagcctggccgccaaaggactggccgaaaatcctctgggcaccaacggcagagcccctaaagctgccagaatgctgaagatggtgtacgactgcaacgtggaaaagaccgccatgatccacgccaagaaatgcgtgttcgagcacagcaaaggccggaaggataccggcgagaacgtgtgggttatgtggcccgctcagaaaaacctgaccgagatggccaccagagccagcaagagttggttcgacgagctgaagaaattcggcgtgccacctgacaacatcctgaccgaaggcctgtggaacagacccaagatggccattggccactacacccagatggtctgggagggcagctacaagcttggatgtggcgtggccacatgcagcgacaagaccctgatcgtgtgccagtatagccctgccggcaactacatcggcagcatcatctatgccatcggcgagccctgcaagaccgacgaggattgcaagtgcgagggctgcaagtgcagcagagatgaggccctgtgcatcaagcccaatcaccaccatcatcaccatcacggcgtgctggcccctcacgattctgttcttcagtaataagcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 21 - protein, 21 aa, synthetic construct
  REGION 1..21: note = Synthetic peptide
MDWTWRVFFLLALATGVHSEN

SEQ ID NO: 22 - protein, 21 aa, synthetic construct
  REGION 1..21: note = Synthetic peptide
AAAHHHHHHHGVLAPHDSVLQ

SEQ ID NO: 23 - DNA, 726 bp, synthetic construct
  misc_feature 1..726: note = Synthetic construct
  source 1..726: mol_type = other DNA
atggactggacttggagagtgttcttcctgctggccctggctacaggcgtgcacagcgagaacatgaacgaccagctgcgggacatcatgctgaacgatcacaacaccctgagaagcctggccgccaaaggactggccgaaaatcctctgggcaccaacggcagagcccctaaagctgccagaatgctgaagatggtgtacgactgcaacgtggaaaagaccgccatgatccacgccaagaaatgcgtgttcgagcacagcaagggcagaaaggacaccggcgagaacgtgtgggttatgtggcccgctcagaaaaacctgaccgagatggccaccagagccagcaagagttggttcgacgagctgaagaaattcggcgtgccacctgacaacatcctgaccgaaggcctgtggaacagacccaagatggccattggccactacacccagatggtctgggagggctcttacaagctcggatgtggcgtggccacatgcagcgacaagaccctgatcgtgtgccagtatagccctgccggcaactacatcggcagcatcatctatgccatcggcgagccctgcaagaccgacgaggattgcaagtgcgagggctgcaagtgcagcagagatgaggccctgtgcatcaagcctaatgccgccgctcaccaccatcatcaccatcaccacggtgttctggcccctcacgattctgttctgcagtaataa

SEQ ID NO: 24 - DNA, 723 bp, synthetic construct
  misc_feature 1..723: note = Synthetic construct
  source 1..723: mol_type = other DNA
atggactggacttggagagtgttcttcctgctggccctggctacaggcgtgcacagcgagaacaacgacgagatggacgagaacaatcccgtggctctgcccggcaagagcaatgtgtctgagccttgccagaccaccaccagcaatcctattcaccctaccaccggcgagcagctggaaaccacaacaggcaagcccagacggaccaccaagggcagaatcctgaaaaccaccaccgtgaagcccatcaagacaacaaagggcaagaagctcaagacgaccaaggggaagctgctgaaaacaacaacagtcaaacccttcaagaccacgaagggaaagccccttaagacaaccaccaaagagcccttccagagcaccacaggtggaccagtgggcccaaccaccgatagatgcaagctgaacaacggcatgaacgaccagctgcgggacatcatgctgaacgatcacaacaccctgagaagcctggccgccaaaggactggccgaaaatcctctgggcaccaacggcagagcccctaaagctgccagaatgctgaagatggtgtacgactgcaacgtggaaaagaccgccatgatccacgccaagaaatgcgtgttcgagcacagcaaaggccggaaggatacaggcgaagccgccgctcaccaccatcatcaccatcaccacggtgttctggcccctcacgattctgttctgcagtaataa

SEQ ID NO: 25 - DNA, 525 bp, synthetic construct
  misc_feature 1..525: note = Synthetic construct
  source 1..525: mol_type = other DNA
atggactggacttggagagtgttcttcctgctggccctggctacaggcgtgcactctgagaacggcagaaaggacaccggcgagaacgtgtgggttatgtggcccgctcagaaaaacctgaccgagatggccaccagagccagcaagagttggttcgacgagctgaagaaattcggcgtgccacctgacaacatcctgaccgaaggcctgtggaacagacccaagatggccattggccactacacccagatggtctgggagggctcttacaagctcggatgtggcgtggccacatgcagcgacaagaccctgatcgtgtgccagtatagccctgccggcaactacatcggcagcatcatctatgccatcggcgagccctgcaagaccgacgaggattgcaagtgcgagggctgcaagtgcagcagagatgaggccctgtgcatcaagcctaatgccgccgctcaccaccatcatcaccatcaccacggtgttctggcccctcacgattctgttctgcagtaataa

SEQ ID NO: 26 - skipped

SEQ ID NO: 27 - protein, 15 aa, synthetic construct
  REGION 1..15: note = Synthetic peptide
NDEMDENNPVALPGK

SEQ ID NO: 28 - protein, 15 aa, synthetic construct
  REGION 1..15: note = Synthetic peptide
ENNPVALPGKSNVSE

SEQ ID NO: 29 - protein, 15 aa, synthetic construct
  REGION 1..15: note = Synthetic peptide
ALPGKSNVSEPCQTT

SEQ ID NO: 30 - protein, 15 aa, synthetic construct
  REGION 1..15: note = Synthetic peptide
SNVSEPCQTTTSNPI

SEQ ID NO: 31 - protein, 15 aa, synthetic construct
  REGION 1..15: note = Synthetic peptide
PCQTTTSNPIHPTTG

SEQ ID NO: 32 - protein, 15 aa, synthetic construct
  REGION 1..15: note = Synthetic peptide
TSNPIHPTTGEQLET

SEQ ID NO: 33 - protein, 15 aa, synthetic construct
  REGION 1..15: note = Synthetic peptide
HPTTGEQLETTTGKP

SEQ ID NO: 34 - protein, 15 aa, synthetic construct
  REGION 1..15: note = Synthetic peptide
EQLETTTGKPRRTTK

SEQ ID NO: 35 - protein, 15 aa, synthetic construct
  REGION 1..15: note = Synthetic peptide
TTGKPRRTTKGRILK

SEQ ID NO: 36 - protein, 15 aa, synthetic construct
  REGION 1..15: note = Synthetic peptide
RRTTKGRILKTTTVK

SEQ ID NO: 37 - protein, 15 aa, synthetic construct
  REGION 1..15: note = Synthetic peptide
GRILKTTTVKPIKTT

SEQ ID NO: 38 - protein, 15 aa, synthetic construct
  REGION 1..15: note = Synthetic peptide
TTTVKPIKTTKGKKL

SEQ ID NO: 39 - protein, 15 aa, synthetic construct
  REGION 1..15: note = Synthetic peptide
PIKTTKGKKLKTTKG

SEQ ID NO: 40 - protein, 15 aa, synthetic construct
  REGION 1..15: note = Synthetic peptide
KGKKLKTTKGKLLKT

SEQ ID NO: 41 - protein, 15 aa, synthetic construct
  REGION 1..15: note = Synthetic peptide
KTTKGKLLKTTTVKP

SEQ ID NO: 42 - protein, 15 aa, synthetic construct
  REGION 1..15: note = Synthetic peptide
KLLKTTTVKPFKTTK

SEQ ID NO: 43 - protein, 15 aa, synthetic construct
  REGION 1..15: note = Synthetic peptide
TTVKPFKTTKGKPLK

SEQ ID NO: 44 - protein, 15 aa, synthetic construct
  REGION 1..15: note = Synthetic peptide
FKTTKGKPLKTTTKE

SEQ ID NO: 45 - protein, 15 aa, synthetic construct
  REGION 1..15: note = Synthetic peptide
GKPLKTTTKEPFQST

SEQ ID NO: 46 - protein, 15 aa, synthetic construct
  REGION 1..15: note = Synthetic peptide
TTTKEPFQSTTGGPV

SEQ ID NO: 47 - protein, 15 aa, synthetic construct
  REGION 1..15: note = Synthetic peptide
PFQSTTGGPVGPTTD

SEQ ID NO: 48 - protein, 15 aa, synthetic construct
  REGION 1..15: note = Synthetic peptide
TGGPVGPTTDRCKLN

SEQ ID NO: 49 - protein, 15 aa, synthetic construct
  REGION 1..15: note = Synthetic peptide
GPTTDRCKLNNGMND

SEQ ID NO: 50 - protein, 15 aa, synthetic construct
  REGION 1..15: note = Synthetic peptide
RCKLNNGMNDQLRDI

SEQ ID NO: 51 - protein, 15 aa, synthetic construct
  REGION 1..15: note = Synthetic peptide
NGMNDQLRDIMLNDH

SEQ ID NO: 52 - protein, 15 aa, synthetic construct
  REGION 1..15: note = Synthetic peptide
QLRDIMLNDHNTLRS

SEQ ID NO: 53 - protein, 15 aa, synthetic construct
  REGION 1..15: note = Synthetic peptide
MLNDHNTLRSLAAKG

SEQ ID NO: 54 - protein, 15 aa, synthetic construct
  REGION 1..15: note = Synthetic peptide
NTLRSLAAKGLAENP

SEQ ID NO: 55 - protein, 15 aa, synthetic construct
  REGION 1..15: note = Synthetic peptide
LAAKGLAENPLGTNG

SEQ ID NO: 56 - protein, 15 aa, synthetic construct
  REGION 1..15: note = Synthetic peptide
LAENPLGTNGRAPKA

SEQ ID NO: 57 - protein, 15 aa, synthetic construct
  REGION 1..15: note = Synthetic peptide
LGTNGRAPKAARMLK

SEQ ID NO: 58 - protein, 15 aa, synthetic construct
  REGION 1..15: note = Synthetic peptide
RAPKAARMLKMVYDC

SEQ ID NO: 59 - protein, 15 aa, synthetic construct
  REGION 1..15: note = Synthetic peptide
ARMLKMVYDCNVEKT

SEQ ID NO: 60 - protein, 15 aa, synthetic construct
  REGION 1..15: note = Synthetic peptide
DELKKFGVPPDNILT

SEQ ID NO: 61 - protein, 15 aa, synthetic construct
  REGION 1..15: note = Synthetic peptide
FGVPPDNILTEGLWN

SEQ ID NO: 62 - protein, 15 aa, synthetic construct
  REGION 1..15: note = Synthetic peptide
DNILTEGLWNRPKMA

SEQ ID NO: 63 - protein, 15 aa, synthetic construct
  REGION 1..15: note = Synthetic peptide
EGLWNRPKMAIGHYT

SEQ ID NO: 64 - protein, 15 aa, synthetic construct
  REGION 1..15: note = Synthetic peptide
RPKMAIGHYTQMVWE

SEQ ID NO: 65 - protein, 15 aa, synthetic construct
  REGION 1..15: note = Synthetic peptide
IGHYTQMVWEGSYKL

SEQ ID NO: 66 - protein, 15 aa, synthetic construct
  REGION 1..15: note = Synthetic peptide
QMVWEGSYKLGCGVA

SEQ ID NO: 67 - protein, 15 aa, synthetic construct
  REGION 1..15: note = Synthetic peptide
GSYKLGCGVATCSDK

SEQ ID NO: 68 - protein, 15 aa, synthetic construct
  REGION 1..15: note = Synthetic peptide
GCGVATCSDKTLIVC

SEQ ID NO: 69 - protein, 15 aa, synthetic construct
  REGION 1..15: note = Synthetic peptide
TCSDKTLIVCQYSPA

SEQ ID NO: 70 - protein, 15 aa, synthetic construct
  REGION 1..15: note = Synthetic peptide
TLIVCQYSPAGNYIG

SEQ ID NO: 71 - protein, 15 aa, synthetic construct
  REGION 1..15: note = Synthetic peptide
QYSPAGNYIGSIIYA

SEQ ID NO: 72 - protein, 15 aa, synthetic construct
  REGION 1..15: note = Synthetic peptide
GNYIGSIIYAIGEPC

SEQ ID NO: 73 - protein, 15 aa, synthetic construct
  REGION 1..15: note = Synthetic peptide
SIIYAIGEPCKTDED

SEQ ID NO: 74 - protein, 15 aa, synthetic construct
  REGION 1..15: note = Synthetic peptide
IGEPCKTDEDCKCEG

SEQ ID NO: 75 - protein, 15 aa, synthetic construct
  REGION 1..15: note = Synthetic peptide
KTDEDCKCEGCKCSR

SEQ ID NO: 76 - protein, 15 aa, synthetic construct
  REGION 1..15: note = Synthetic peptide
CKCEGCKCSRDEALC

SEQ ID NO: 77 - protein, 14 aa, synthetic construct
  REGION 1..14: note = Synthetic peptide
CKCSRDEALCIKPN

SEQ ID NO: 78 - protein, 25 aa, synthetic construct
  REGION 1..25: note = Synthetic peptide
PAQKNLTEMATRASKSWFDELKKFG

SEQ ID NO: 79 - protein, 45 aa, synthetic construct
  REGION 1..45: note = Synthetic peptide
CVFEHSKGRKDTGENVWVMWPAQKNLTEMATRASKSWFDELKKFG

SEQ ID NO: 80 - protein, 50 aa, synthetic construct
  REGION 1..50: note = Synthetic peptide
CPAQKNLTEMATRASKSWFDELKKFGVPPDNILTEGLWNRPKMAIGHYTQ

SEQ ID NO: 81 - protein, 5 aa, synthetic construct
  REGION 1..5: note = Synthetic peptide
SKSWF

SEQ ID NO: 82 - protein, 10 aa, synthetic construct
  REGION 1..10: note = Synthetic peptide
MATRASKSWF

SEQ ID NO: 83 - protein, 10 aa, synthetic construct
  REGION 1..10: note = Synthetic peptide
SKSWFDELKK

SEQ ID NO: 84 - protein, 5 aa, synthetic construct
  REGION 1..5: note = Synthetic peptide
AMIHA

SEQ ID NO: 85 - protein, 10 aa, synthetic construct
  REGION 1..10: note = Synthetic peptide
NVEKTAMIHA

SEQ ID NO: 86 - protein, 10 aa, synthetic construct
  REGION 1..10: note = Synthetic peptide
AMIHAKKCVF

SEQ ID NO: 87 - protein, 5 aa, synthetic construct
  REGION 1..5: note = Synthetic peptide
ENVWV

SEQ ID NO: 88 - protein, 10 aa, synthetic construct
  REGION 1..10: note = Synthetic peptide
RKDTGENVWV

SEQ ID NO: 89 - protein, 10 aa, synthetic construct
  REGION 1..10: note = Synthetic peptide
ENVWVMWPAQ

SEQ ID NO: 90 - protein, 15 aa, synthetic construct
  REGION 1..15: note = Synthetic peptide
LTEMATRASKSWFDE

SEQ ID NO: 91 - protein, 15 aa, synthetic construct
  REGION 1..15: note = Synthetic peptide
ATEMATRASKSWFDE

SEQ ID NO: 92 - protein, 15 aa, synthetic construct
  REGION 1..15: note = Synthetic peptide
LAEMATRASKSWFDE

SEQ ID NO: 93 - protein, 15 aa, synthetic construct
  REGION 1..15: note = Synthetic peptide
LTAMATRASKSWFDE

SEQ ID NO: 94 - protein, 15 aa, synthetic construct
  REGION 1..15: note = Synthetic peptide
LTEAATRASKSWFDE

SEQ ID NO: 95 - protein, 15 aa, synthetic construct
  REGION 1..15: note = Synthetic peptide
LTEMGTRASKSWFDE

SEQ ID NO: 96 - protein, 15 aa, synthetic construct
  REGION 1..15: note = Synthetic peptide
LTEMAARASKSWFDE

SEQ ID NO: 97 - protein, 15 aa, synthetic construct
  REGION 1..15: note = Synthetic peptide
LTEMATAASKSWFDE

SEQ ID NO: 98 - protein, 15 aa, synthetic construct
  REGION 1..15: note = Synthetic peptide
LTEMATRGSKSWFDE

SEQ ID NO: 99 - protein, 15 aa, synthetic construct
  REGION 1..15: note = Synthetic peptide
LTEMATRAAKSWFDE

SEQ ID NO: 100 - protein, 15 aa, synthetic construct
  REGION 1..15: note = Synthetic peptide
LTEMATRASASWFDE

SEQ ID NO: 101 - protein, 15 aa, synthetic construct
  REGION 1..15: note = Synthetic peptide
LTEMATRASKAWFDE

SEQ ID NO: 102 - protein, 15 aa, synthetic construct
  REGION 1..15: note = Synthetic peptide
LTEMATRASKSAFDE

SEQ ID NO: 103 - protein, 15 aa, synthetic construct
  REGION 1..15: note = Synthetic peptide
LTEMATRASKSWADE

SEQ ID NO: 104 - protein, 15 aa, synthetic construct
  REGION 1..15: note = Synthetic peptide
LTEMATRASKSWFAE

SEQ ID NO: 105 - protein, 15 aa, synthetic construct
  REGION 1..15: note = Synthetic peptide
LTEMATRASKSWFDA

SEQ ID NO: 106 - protein, 15 aa, synthetic construct
  REGION 1..15: note = Synthetic peptide
TRASKSWFDELKKFG

SEQ ID NO: 107 - protein, 15 aa, synthetic construct
  REGION 1..15: note = Synthetic peptide
ARASKSWFDELKKFG

SEQ ID NO: 108 - protein, 15 aa, synthetic construct
  REGION 1..15: note = Synthetic peptide
TAASKSWFDELKKFG

SEQ ID NO: 109 - protein, 15 aa, synthetic construct
  REGION 1..15: note = Synthetic peptide
TRGSKSWFDELKKFG

SEQ ID NO: 110 - protein, 15 aa, synthetic construct
  REGION 1..15: note = Synthetic peptide
TRAAKSWFDELKKFG

SEQ ID NO: 111 - protein, 15 aa, synthetic construct
  REGION 1..15: note = Synthetic peptide
TRASASWFDELKKFG

SEQ ID NO: 112 - protein, 15 aa, synthetic construct
  REGION 1..15: note = Synthetic peptide
TRASKAWFDELKKFG

SEQ ID NO: 113 - protein, 15 aa, synthetic construct
  REGION 1..15: note = Synthetic peptide
TRASKSAFDELKKFG

SEQ ID NO: 114 - protein, 15 aa, synthetic construct
  REGION 1..15: note = Synthetic peptide
TRASKSWADELKKFG

SEQ ID NO: 115 - protein, 15 aa, synthetic construct
  REGION 1..15: note = Synthetic peptide
TRASKSWFAELKKFG

SEQ ID NO: 116 - protein, 15 aa, synthetic construct
  REGION 1..15: note = Synthetic peptide
TRASKSWFDALKKFG

SEQ ID NO: 117 - skipped

SEQ ID NO: 118 - protein, 157 aa, synthetic construct
  REGION 1..157: note = Synthetic peptide
MDWTWRVFFLLALATGVHSENMNDQLRDIMLNDHNTLRSLAAKGLAENPLGTNGRAPKAARMLKMVYDCNVEKTAMIHAKKCVFEHSKGRKDTGENVWVMWPAQKNLTEMATRASKSWFDELKKFGVPPDNILTEGLWNRPKMAIGHYTQHHHHHHH

SEQ ID NO: 119 - protein, 108 aa, synthetic construct
  REGION 1..108: note = Synthetic peptide
MDWTWRVFFLLALATGVHSENCVFEHSKGRKDTGENVWVMWPAQKNLTEMATRASKSWFDELKKFGVPPDNILTEGLWNRPKMAIGHYTQMVWEGSYKLGCHHHHHHH

SEQ ID NO: 120 - protein, 158 aa, synthetic construct
  REGION 1..158: note = Synthetic peptide
MDWTWRVFFLLALATGVHSENCGRKDTGENVWVMWPAQKNLTEMATRASKSWFDELKKFGVPPDNILTEGLWNRPKMAIGHYTQMVWEGSYKLGSGVATSSDKTLIVSQYSPAGNYIGSIIYAIGEPSKTDEDSKSEGSKSSRDEALSIKPNHHHHHH

SEQ ID NO: 121 - protein, 133 aa, synthetic construct
  REGION 1..133: note = Synthetic peptide
MDWTWRVFFLLALATGVHSENCGRKDTGENVWVMWPAQKNLTEMATRASKSWFDELKKFGVPPDNILTEGLWNRPKMAIGHYTQMVWEGSYKLGSGVATSSDKTLIVSQYSPAGNYIGSIIYAIGEPHHHHHH

SEQ ID NO: 122 - protein, 115 aa, synthetic construct
  REGION 1..115: note = Synthetic peptide
MDWTWRVFFLLALATGVHSENCGRKDTGENVWVMWPAQKNLTEMATRASKSWFDELKKFGVPPDNILTEGLWNRPKMAIGHYTQMVWEGSYKLGSGVATSSDKTLIVSQHHHHHH

SEQ ID NO: 123 - protein, 100 aa, synthetic construct
  REGION 1..100: note = Synthetic peptide
MDWTWRVFFLLALATGVHSENCGRKDTGENVWVMWPAQKNLTEMATRASKSWFDELKKFGVPPDNILTEGLWNRPKMAIGHYTQMVWEGSYKLGHHHHHH

SEQ ID NO: 124 - protein, 339 aa, synthetic construct; identical to SEQ ID NO: 2
  REGION 1..339: note = Synthetic peptide